(19) United States
(12) Patent Application Publication
(10) Pub. No.: US 2026/0335249 A1
(43) Pub. Date: Oct. 29, 2026

(54) Branched Lipid Compositions, Lipid Nanoparticles (LNPs) Comprising the Same, and Methods of UseThereof

(71) Applicant: The Trustees of the University of Pennsylvania, Philadelphia, PA (US)

(72) Inventors: Michael J. Mitchell, Philadelphia, PA (US); Marshall Scott Padilla, Philadelphia, PA (US)

(21) Appl. No.: 19/107,296

(22) PCT Filed: Aug. 29, 2023
(86) PCT No.: PCT/US2023/073072

§ 371 Date: Feb. 27, 2025

Related U.S. Application Data
(60) Provisional application No. 63/373,793, filed on Aug. 29, 2022.

(51) Int. Cl. A61K 9/51; A61K 9/00; A61K 31/7088; A61K 38/17; A61K 38/46; A61K 45/06; A61K 48/00; A61P 35/00; C07D 295/13; C12N 5/0783

(52) U.S. Cl. CPC A61K 9/5123; A61K 9/0053; A61K 31/7088; A61K 38/1774; A61K 38/465; A61K 45/06; A61K 48/0033; A61P 35/00; C07D 295/13; C12N 5/0636; C12N 5/0646; C12N 2510/00

(57) ABSTRACT

The present disclosure relates, in one aspect, to ionizable lipids of Formula (I). In another aspect, the present disclosure relates to the preparation of ionizable lipids of Formula (I). In other aspects, the present disclosure relates to lipid nanoparticles (LNPs) comprising at least one ionizable lipid of Formula (I), at least one neutral lipid, cholesterol, and at least one conjugated lipid, and methods of use thereof.

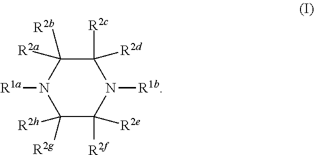

### CROSS-REFERENCE TO RELATED APPLICATIONS

This application claims priority under 35 U.S.C. § 119(e) to U.S. Provisional Patent Application No. 63/373,793, filed Aug. 29, 2022, which is incorporated herein by reference in its entirety.

### STATEMENT REGARDING FEDERALLY SPONSORED RESEARCH OR DEVELOPMENT

This invention was made with government support under TR002776 awarded by the National Institutes of Health. The government has certain rights in the invention.

### SEQUENCE LISTING

The XML file named “046483-7399WO1—Sequence Listing.xml” created on Aug. 24, 2023, comprising 3.8 Kbytes, is hereby incorporated by reference in its entirety.

### BACKGROUND

In recent years, messenger RNA (mRNA), a transient intermediate between genes and proteins, has emerged as a promising new approach for therapeutic applications including protein replacement therapies, vaccines, and gene editing. However, mRNA rapidly degrades and cannot easily cross the cell membrane due to its large size and negative charge.

Therefore, the use of mRNA therapeutics, among other cargo (e.g., DNA, RNA, small molecules, and/or polypeptides) requires safe, effective, and stable delivery systems to protect from degradation and allow cellular uptake and functionality.

Thus, there is a need in the art for lipid nanoparticles (LNPs) suitable for delivery of cargo to a target, ionizable lipids for the preparation thereof, and methods using the same. The present disclosure addresses these needs.

### BRIEF SUMMARY

In one aspect, the present disclosure provides certain branched ionizable lipid (IL) compounds, methods of preparation thereof, lipid nanoparticles (LNPs) comprising the same, and methods of use thereof for delivery of cargo (e.g., mRNA).

In one aspect, the present disclosure provides a compound of Formula (I), or a salt, solvate, stereoisomer, or isotopologue thereof, wherein R^{1a}, R^{1b}, R^{2a}, R^{2b}, R^{2c}, R^{2d}, R^{2e}, R^{2f}, R^{2g}, and R^{2h} are defined elsewhere herein:

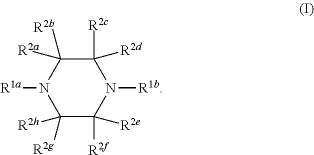

In another aspect, the present disclosure provides a lipid nanoparticle (LNP) composition comprising at least one ionizable lipid compound of Formula (I), or a salt, solvate, stereoisomer, or isotopologue thereof. In certain embodiments, the LNP comprises at least one neutral lipid. In certain embodiments, the LNP comprises cholesterol. In certain embodiments, the LNP comprises at least one conjugated lipid. In certain embodiments, the LNP further comprises at least one cargo molecule.

In another aspect, the present disclosure provides a pharmaceutical composition comprising at least one LNP of the present disclosure and a pharmaceutically acceptable carrier.

In another aspect, the present disclosure provides a method of treating, preventing, and/or ameliorating a disease in a subject. In certain embodiments, the method comprises administering to the subject at least one lipid nanoparticle (LNP) of the present disclosure and/or at least one pharmaceutical composition of the present disclosure.

In another aspect, the present disclosure provides a method of delivering a nucleic acid or therapeutic agent to the liver of a subject. In certain embodiments, the method comprises administering to the subject at least one lipid nanoparticle (LNP) of the present disclosure and/or at least one pharmaceutical composition of the present disclosure.

In another aspect, the present disclosure provides a method of preparing a modified immune cell or precursor thereof. In certain embodiments, the method comprises contacting an immune cell or precursor thereof with at least one lipid nanoparticle (LNP) of the present disclosure and/or at least one pharmaceutical composition of the present disclosure.

### BRIEF DESCRIPTION OF THE FIGURES

The drawings illustrate generally, by way of example, but not by way of limitation, various embodiments of the present application.

FIG. 1 depicts non-limiting embodiments of the synthesis of epoxide intermediates used in the preparation of the ionizable lipid compositions of the present disclosure.

FIGS. 2A-2B depict non-limiting embodiments of the synthesis of the ionizable lipid compositions of the present disclosure by SN2 addition of exemplary polyamine cores to exemplary epoxide substrates.

FIGS. 3A-3H: in vitro and in vivo fluc mRNA delivery of LNPs comprising exemplary linear and branched LNPs prepared from polyamine core 494. FIGS. 3A-3B: LNPs with 494-core ILs and encapsulating fluc mRNA were first incubated in HeLa cells at 20 ng of mRNA per 20,000 cells. After 24 h, luminescence (FIG. 3A) and cell viability (FIG. 3B) were evaluated. Normalized luciferase expression is reported as mean±SEM of n=3 biological replicates, averaged from n=4 technical replicates each. Percent cell viability was nonmalized to untreated cells and is reported as mean f SEM of n=3 biological replicates, averaged from n=3 technical replicates each. FIGS. 3C-3F: C57BL/6J were intravenously injected with the LNPs at 0.1 mg/kg of mRNA. After 12 h, full body luminescence (FIG. 3C) and images were obtained via IVIS (FIG. 3D). The mice were then sacrificed and organ luminescence (FIG. 3E) and images were collected via IVIS (FIG. 3F). Total flux is reported as mean±SEM of n=3. Two-way ANOVA with post hoc Student's t tests using the Holm-Sidak correction for multiple comparisons were used to compare normalized luciferase expression across branching groups and linker lengths (FIG. 3A). One-way ANOVA with post hoc Student's t tests using the Holm-Šidák correction for multiple comparisons were used to compare either cell viability across treatment groups to the untreated group (FIG. 3C) or total flux across treatment groups (FIG. 3C and FIG. 3E) to C12-200. *p≤0.05, **p≤0.01, ***p≤0.001, ****p≤0.0001.

FIGS. 4A-4E: in vitro and in vivo fluc mRNA delivery of LNPs comprising exemplary linear and branched LNPs prepared from polyamine core 200. FIGS. 4A-4B: exemplary linear and branched LNPs with 200-core ILs that encapsulated fluc mRNA were first incubated in HeLa cells at 20 ng of mRNA per 20,000 cells. After 24 h, luminescence (FIG. 4A) and cell viability (FIG. 4B) were evaluated. Normalized luciferase expression is reported as mean±SEM of n=3 biological replicates, averaged from n=4 technical replicates each. Percent cell viability was normalized to untreated cells and is reported as mean±SEM of n=3 biological replicates, averaged from n=3 technical replicates each. FIGS. 4C-4E: C57BL/J were intravenously injected with the LNPs at 0.1 mg/kg of mRNA. After 12 h, full body luminescence was obtained via IVIS (FIG. 4C). The mice were then sacrificed and organ luminescence (FIG. 4D) and images were collected via IVIS (FIG. 4E). Total flux is reported as mean f SEM of n=3. Two-way ANOVA with post hoc Student's t tests using the Holm-Šidák correction for multiple comparisons were used to compare normalized luciferase expression across branching groups and linker lengths (FIG. 4A). One-way ANOVA with post hoc Student's t tests using the Holm-Šidák correction for multiple comparisons were used to compare either cell viability across treatment groups to the untreated group (FIG. 4B) or total flux (FIGS. 4C-D) across treatment groups to C12-200. *p<0.05, **p≤0.01, ***p≤0.001, ****p≤0.0001.

FIGS. 5A-5D provide bar graphs depicting combined organ (i.e., liver, spleen, lungs, kidneys, and heart) luminescence data for Black 6 mice which were administered exemplary LNPs of the present disclosure, including LNPs comprising ionizable lipids prepared using polyamine core 494 (FIG. 5A) and polyamine core 200 (FIG. 5B), as compared to selected controls (e.g., LNPs comprising C8, C12, and/or MC3 ionizable lipids), wherein the LNPs comprise TriLink Luciferase mRNA encapsulated therein. FIGS. 5C-5D provides a magnified images of the data presented in FIGS. 5A-5B, respectively, such that the data for organs other than the liver (i.e., heart, kidneys, lungs, and spleen) are shown.

FIGS. 6A-6C: eight exemplary LNPs comprising ionizable lipids prepared from polyamine core 200 were analyzed for liver TTR knockdown. The LNPs were reformulated with Cas9 mRNA and TTR single guide RNA (sgRNA). The mice were injected at a dose of 1.0 mg of combined RNA per kg of body weight (FIG. 6A). After 7 days, blood was extracted and serum TTR levels were measured via an ELISA to serum before injecting (FIG. 6B). Additionally, livers were extracted and indels were measured via next generation sequencing. Overall, branched LNPs seem to significantly enhance Cas9-mediated gene editing (FIG. 6C).

FIGS. 7A-7N: analysis of physiochemical characteristics, morphology, and stability of certain exemplary LNPs comprising unbranched and branched ILs. The in vivo liver luminescence of eight exemplary LNPs comprising linear and branched ILs prepared from polyamine core 200 were plotted and fitted using a cubic least squares regression against hydrodynamic diameter (FIG. 7A), PDI (FIG. 7B), ζ-potential (FIG. 7C), pKa (FIG. 7D), encapsulation efficiency (FIG. 7E), and HeLa cell luminescence (FIG. 7F). FIGS. 7G-J: cryo-TEM images of C8-200 (FIG. 7G), E4i-200 (FIG. 7H), E4t-200 (FIG. 7I), and E4s-200 (FIG. 7J). FIGS. 7K-7N: the stability of eight exemplary LNPs comprising ILs prepared from polyamine core 200 was evaluated by measuring the hydrodynamic diameter and PDI every hour for 24 h immediately after incubating them at 37° C. in 1×PBS (FIGS. 7K-7L) or DMEM supplemented with 10% FBS (FIGS. 7M-7N).

FIGS. 8A-8G: exemplary data for LNPs comprising branched and linear ILs in the liver and artificial endosomes. C57BL/6J mice were injected intravenously with 1.0 mg of clodronate. After 24 h, the mice were reinjected intravenously with eight exemplary LNPs comprising ILs prepared from polyamine core 200, wherein the LNPs further comprise fluc mRNA at a dose of 0.1 mg/kg. FIG. 8A: mice were sacrificed and dissected after a total of 36 h and liver luminescence was measured via IVIS. Total flux is reported as mean±SEM of n=3. Apolipoprotein E (APOE) knockout mice were injected intravenously with four exemplary LNPs containing fluc mRNA at a dose of 0.1 mg/kg (FIG. 8B). The mice were sacrificed and dissected after 12 h and liver luminescence was measured via IVIS. Total flux is reported as mean±SEM of n=3. FIGS. 8C-8D: C57BL/6J mice were injected with eight 200-core LNPs containing 1 mol % DiR, and after 12 h, the mice were then sacrificed and organ fluorescence (FIG. 8C) and images (FIG. 8D) were collected. Total radiant efficiency is reported as mean±SEM of n=3. FIGS. 8F-8G: eight exemplary representative LNPs were mixed with artificial endosomes containing a FRET pair. Upon mixing, the percentage of endosome disruption was measured by examining increases in donor fluorescence, which was performed at several time points. The branched LNPs significantly increase endosome disruption compared to the linear LNPs (endosome molar ratio: DOPS (25%), DOPC (25%), DOPE (48%), NBD-PE (1%), and Rho-PE (1%).

FIGS. 9A-9B: exemplary toxicity (FIG. 9A) and luciferase expression (FIG. 9B) data in CAL-27 cells with administration of certain exemplary LNPs of the present disclosure comprising luciferase mRNA. LNPs were incubated with luciferase mRNA and were treated onto the cells at a dose of 20 ng of mRNA per 20,000 cells. After 24 h, a luciferase assay and cell viability assay were performed to analyze luciferase expression and toxicity, respectively.

FIGS. 10A-10B: exemplary tumor luminescence data using a CAL-27 tumor-induced mouse model with administration of certain exemplary LNPs of the present disclosure comprising luciferase. One million CAL-27 cells were inoculated on the right flanks of Nu/J mice, and after tumors grew for two weeks, five LNPs encapsulating luciferase mRNA, including two branched LNPs and three linear LNPs including C12-200, were injected intratumorally at a dose of 0.1 mg/kg. PBS was injected into one group as a control. After 6 h, the mice were sacrificed, and the major organs and tumors were imaged for luminescence.

FIGS. 11A-11D: exemplary viability data for certain squamous cell carcinoma cell lines with administration of exemplary LNPs of the present disclosure comprising human p53 mRNA and/or luciferase mRNA. CAL-27 cells (FIGS. 11A-11B) and OECM-1 cells (FIGS. 11C-11D), were treated with an E10i-494 LNP encapsulating human p53 mRNA or luciferase mRNA. The cells were dosed at varying intervals and the overall viability was measured after 24 h (FIGS. 11A and 11C) and 48 h (FIGS. 11B and 11D) using a cellular toxicity assay.

FIGS. 12A-12B depict an initial potency evaluation of exemplary LNPs of the present disclosure by measurement of relative luminescence (FIG. 12A) and viability (FIG. 12B) in induced pluripotent stem cells (iPSC-SV20) after 24 h, wherein exemplary LNPs comprise 20 ng of luciferase mRNA per 15,000 cells.

FIGS. 13A-13B depict a dose escalation study of exemplary LNPs of the present disclosure comprising luciferase mRNA in iPSC-SV20 cells, wherein luminescence (FIG. 13A) and viability (FIG. 13B) were measured as a function of mRNA dose (e.g., 20, 50, 100, 200, 400, and 600 ng/well).

FIGS. 14A-14H: exemplary data demonstrating successful transfection of induced pluripotent stem cells (iPSC-SV20) with mCherry mRNA with administration of exemplary LNPs of the present disclosure comprising mCherry mRNA cargo. A total of 90,000 iPSC-SV20 cells were plated in 24-well plates overnight until they reached 50% confluent. They were treated with different concentrations of exemplary LNPs of the present disclosure and were harvested after 24 h. Flow cytometry was performed to measure the percentage of cells that were successfully transfected (FIGS. 14A-14F). Cells were further imaged by fluorescence microscopy to visualize mCherry signal (FIGS. 14G-14H).

FIGS. 15A-15H provide exemplary fluorescence-activated cell sorting (FACS) flow cytometry graphs of induced pluripotent stem cells (iPSCs) transfected with mCherry mRNA with administration of a non-limiting exemplary LNP (E4i-200) of the present disclosure comprising mCherry mRNA cargo. A total of 50,000 iPSCs were plated in 24-well plates overnight and were then dosed with 366 ng of mCherry mRNA encapsulated in the E4i-200 LNP (FIGS. 15E-15H) or control (FIGS. 15A-15D). Cells were harvested after 24 h (FIGS. 15A and 15E), 48 h (FIGS. 15B and 15F), 72 h (FIGS. 15C and 15G), and 96 h (FIGS. 15D and 15H).

FIGS. 16A-16B provide bar graphs depicting relative luminescence data (FIG. 16A) and viability data (FIG. 16B) for activated primary T cells (1:1 CD4+:CD8+) from healthy human donors, which were incubated with exemplary LNPs of the present disclosure for 24 h, including LNPs comprising ILs prepared using polyamine core 494 and polyamine core 200, as compared to selected controls (e.g., LNPs comprising C8, C12, and/or MC3 ionizable lipids), wherein the LNPs comprise luciferase mRNA encapsulated therein (200 ng luciferase mRNA per 60,000 cells).

FIGS. 17A-17B provide bar graphs depicting relative luminescence data (FIG. 17A) and viability data (FIG. 17B) for activated primary T cells (1:1 CD4+:CD8+) from healthy human donors, which were incubated with exemplary LNPs of the present disclosure for 24 h, wherein the LNPs comprise luciferase mRNA encapsulated therein (200 ng luciferase mRNA per 60,000 cells), and wherein all LNPs were formulated with a ratio of ionizable lipid:DOPE:cholesterol:CT4PEG 2000 of 40:30:25:2.5.

FIG. 18 provides a bar graph depicting relative luminescence data of NK-92MI cells (i.e., immortalized human NK cells that self-express IL-2) which were incubated with certain exemplary LNPs of the present disclosure for 24 h, as compared to controls, wherein the LNPS comprise luciferase mRNA.

### DETAILED DESCRIPTION OF THE INVENTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Description

Lipid nanoparticles (LNPs) have emerged as the preeminent drug delivery vehicle for mRNA therapy. One major factor for the success of LNPs over previous lipid-based particles is the transition from a permanently cationic lipid that can induce tremendous toxicity, to an ionizable lipid (IL) that is only cationic at low pH conditions. Immense effort has been placed on optimizing IL structure, which contains an amine core conjugated to long lipid tails, as small molecular adjustments can result in drastic changes in the overall efficacy of the resulting LNP.

In one aspect, the present disclosure relates to the design and evaluation of LNPs comprising ionizable lipids comprising lipid tails with terminal branching. A simple and modular synthetic scheme was developed for exemplary lipids described herein which enables facile adjustment of length and terminal branching. Further, the present disclosure describes application of this synthetic scheme utilizing certain exemplary polyamine cores to prepare non-limiting ILs.

In one aspect, the present disclosure relates to the observation that lipid branching significantly increases liver transfection compared to non-branched lipids, including in a gene editing model. Further, utilizing an array of experiments involving physicochemical evaluation and liver cell targeting, it was determined that lipid branching induces greater endosomal escape of mRNA.

The present disclosure further provides non-limiting, exemplary applications of the LNPs described herein comprising branched ILs, including mRNA delivery for mRNA therapy, liver gene editing, stem cell reprogramming, and CAR T and/or CAR NK cell therapy.

### Definitions

The term “about” as used herein can allow for a degree of variability in a value or range, for example, within 10%, within 5%, or within 1% of a stated value or of a stated limit of a range, and includes the exact stated value or range.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “adjuvant” as used herein is defined as any molecule to enhance an antigen-specific adaptive immune response.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C═C(CH₃), —C≡C(CH₂CH₃), —CH₂C≡CH, —CH₂C≡C(CH₃), and —CH₂C≡C(CH₂CH₃) among others.

The term “alkylene” or “alkylenyl” as used herein refers to a bivalent saturated aliphatic radical (e.g., —CH₂—, —CH₂CH₂—, and —CH₂CH₂CH₂—, inter alia). In certain embodiments, the term may be regarded as a moiety derived from an alkene by opening of the double bond or from an alkane by removal of two hydrogen atoms from the same (e.g., —CH₂—) different (e.g., —CH₂CH₂—) carbon atoms. Similarly, the terms “heteroalkylenyl”, “cycloalkylenyl”, “heterocycloalkylenyl”, and the like, as used herein refer to a divalent radical of the moiety corresponding to the base group (e.g., heteroalkyl, cycloalkyl, and/or heterocycloalkyl). A divalent radical possesses two open valencies at any position(s) of the group. Thus, the divalent radical may form a single bond to two distinct atoms or groups, or may form a double bond with one atom.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “anionic lipid” refers to any lipid that is negatively charged at physiological pH. These lipids include phosphatidylglycerol, cardiolipin, diacylphosphatidylserine, diacylphosphatidic acid, N-dodecanoylphosphatidylethanolamines, N-succinylphosphatidylethanolamines, N-glutarylphosphatidylethanolamines, lysylphosphatidylglycerols, palmitovloleyolphosphatidylglycerol (POPG), and other anionic modifying groups joined to neutral lipids.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “atm” as used herein refers to a pressure in atmospheres under standard conditions. Thus, 1 atm is a pressure of 101 kPa, 2 atm is a pressure of 202 kPa, and so on.

The term “cationic lipid” refers to any of a number of lipid species that carry a net positive charge at a selected pH, such as physiological pH (e.g., pH of about 7.0). It has been found that cationic lipids comprising alkyl chains with multiple sites of unsaturation, e.g., at least two or three sites of unsaturation, are particularly useful for forming lipid particles with increased membrane fluidity. A number of cationic lipids and related analogs, which are also useful in the present disclosure, have been described in U.S. Patent Publication Nos. 20060083780 and 20060240554; U.S. Pat. Nos. 5,208,036; 5,264,618; 5,279,833; 5,283,185; 5,753,613; and 5,785,992; and PCT Publication No. WO 96/10390, the disclosures of which are herein incorporated by reference in their entirety for all purposes. Non-limiting examples of cationic lipids are described in detail herein. In some cases, the cat-ionic lipids comprise a protonatable tertiary amine (e.g., pH titratable) head group, Cis alkyl chains, ether linkages between the head group and alkyl chains, and 0 to 3 double bonds. Such lipids include, e.g., DSDMA, DLinDMA, DLenDMA, and DODMA.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4- 2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The terms “epoxy-functional” or “epoxy-substituted” as used herein refers to a functional group in which an oxygen atom, the epoxy substituent, is directly attached to two adjacent carbon atoms of a carbon chain or ring system. Examples of epoxy-substituted functional groups include, but are not limited to, 2,3-epoxypropyl, 3,4-epoxybutyl, 4,5-epoxypentyl, 2,3-epoxypropoxy, epoxypropoxypropyl, 2-glycidoxyethyl, 3-glycidoxypropyl, 4-glycidoxybutyl, 2-(giycidoxycarbonyl)propyl, 3-(3,4-epoxycylohexyl)propyl, 2-(3,4-epoxycyclohexyl)ethyl, 2-(2,3-epoxycylopentyl)ethyl, 244-methyl-34-epoxycyclohexy)propyl, 2-(3,4-epoxy-3-methylcylohexyl)-2-methylethyl, and 5,6-epoxyhexyl.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

In particular, in the case of a mRNA, and “effective amount” or “therapeutically effective amount” of a therapeutic nucleic acid as relating to a mRNA is an amount sufficient to produce the desired effect, e.g., mRNA-directed expression of an amount of a protein that causes a desirable biological effect in the organism within which the protein is expressed. For example, in some embodiments, the expressed protein is an active form of a protein that is normally expressed in a cell type within the body, and the therapeutically effective amount of the mRNA is an amount that produces an amount of the encoded protein that is at least 50% (e.g., at least 60%, or at least 70%, or at least 80%, or at least 90%) of the amount of the protein that is normally expressed in the cell type of a healthy individual. For example, in some embodiments, the expressed protein is a protein that is normally expressed in a cell type within the body, and the therapeutically effective amount of the mRNA is an amount that produces a similar level of expression as observed in a healthy individual in an individual with aberrant expression of the protein (i.e., protein deficient individual). Suitable assays for measuring the expression of an mRNA or protein include, but are not limited to dot blots, Northern blots, in situ hybridization, ELISA, immunoprecipitation, enzyme function, as well as phenotypic assays known to those of skill in the art.

The term “encode” as used herein refers to the product specified (e.g., protein and RNA) by a given sequence of nucleotides in a nucleic acid (i.e., DNA and/or RNA), upon transcription or translation of the DNA or RNA, respectively. In certain embodiments, the term “encode” refers to the RNA sequence specified by transcription of a DNA sequence. In certain embodiments, the term “encode” refers to the amino acid sequence (e.g., polypeptide or protein) specified by translation of mRNA. In certain embodiments, the term “encode” refers to the amino acid sequence specified by transcription of DNA to mRNA and subsequent translation of the mRNA encoded by the DNA sequence. In certain embodiments, the encoded product may comprise a direct transcription or translation product. In certain embodiments, the encoded product may comprise post-translational modifications understood or reasonably expected by one skilled in the art.

The term “fully encapsulated” indicates that the active agent or therapeutic agent in the lipid particle is not significantly degraded after exposure to serum or a nuclease or protease assay that would significantly degrade free DNA, RNA, or protein. In a fully encapsulated system, preferably less than about 25% of the active agent or therapeutic agent in the particle is degraded in a treatment that would normally degrade 100% of free active agent or therapeutic agent, more preferably less than about 10%, and most preferably less than about 5% of the active agent or therapeutic agent in the particle is degraded. In the context of nucleic acid therapeutic agents, full encapsulation may be determined by an OLIGREEN® assay. OLIGREEN® is an ultra-sensitive fluorescent nucleic acid stain for quantitating oligonucleotides and single-stranded DNA or RNA in solution (available from Invitrogen Corporation; Carlsbad, Calif.). “Fully encapsulated” also indicates that the lipid particles are serum stable, that is, that they do not rapidly decompose into their component parts upon in vivo administration.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl. N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl. 2-benzothiazolyl, 4-benzothiazolyl, 5-benzothiazolyl. 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-vl), and the like.

The term “heteroarylalkyl” as used herein refers to alkyl groups as defined herein in which a hydrogen or carbon bond of an alkyl group is replaced with a bond to a heteroaryl group as defined herein.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “ionizable lipid” as used herein refers to a lipid (e.g., a cationic lipid) having at least one protonatable or deprotonatable group, such that the lipid is positively charged at a pH at or below physiological pH (e.g., pH 7.4), and neutral at a second pH, preferably at or above physiological pH. It will be understood by one of ordinary skill in the art that the addition or removal of protons as a function of pH is an equilibrium process, and that the reference to a charged or neutral lipid refers to the nature of the predominant species and does not require that all of the lipid be present in the charged or neutral form. Generally, ionizable lipids have a pKₐ of the protonatable group in the range of about 4 to about 7.

The term “local delivery,” as used herein, refers to delivery of an active agent or therapeutic agent such as a messenger RNA directly to a target site within an organism. For example, an agent can be locally delivered by direct injection into a disease site such as a tumor or other target site such as a site of inflammation or a target organ such as the liver, heart, pancreas, kidney, and the like.

The term “lipid” refers to a group of organic compounds that include, but are not limited to, esters of fatty acids and are characterized by being insoluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids.

The term “lipid conjugate” refers to a conjugated lipid that inhibits aggregation of lipid particles. Such lipid conjugates include, but are not limited to, polyamide oligomers (e.g., ATTA-lipid conjugates), PEG-lipid conjugates, such as PEG coupled to dialkyloxypropyls, PEG coupled to diacylglycerols, PEG coupled to cholesterol, PEG coupled to phosphatidylethanolamines, PEG conjugated to ceramides (e.g., U.S. Pat. No. 5,885,613, the disclosure of which is herein incorporated by reference in its entirety for all purposes), cationic PEG lipids, and mixtures thereof. PEG can be conjugated directly to the lipid or may be linked to the lipid via a linker moiety. Any linker moiety suitable for coupling the PEG to a lipid can be used including, e.g., non-ester containing linker moieties and ester-containing linker moieties. In preferred embodiments, non-ester containing linker moieties are used.

As used herein, “lipid encapsulated” can refer to a lipid particle that provides an active agent or therapeutic agent, such as a nucleic acid (e.g., a protein cargo), with full encapsulation, partial encapsulation, or both. In a preferred embodiment, the nucleic acid is fully encapsulated in the lipid particle (e.g., to form an SPLP, pSPLP, SNALP, or other nucleic acid-lipid particle).

The term “lipid nanoparticle” refers to a particle having at least one dimension on the order of nanometers (e.g., 1-1,000 nm) which includes one or more lipids and/or additional agents.

The term “lipid particle” is used herein to refer to a lipid formulation that can be used to deliver an active agent or therapeutic agent, such as a nucleic acid (e.g., mRNA), to a target site of interest. In the lipid particle of the disclosure, which is typically formed from a cationic lipid, a non-cationic lipid, and a conjugated lipid that prevents aggregation of the particle, the active agent or therapeutic agent may be encapsulated in the lipid, thereby protecting the agent from enzymatic degradation.

The term “monovalent” as used herein refers to a substituent connecting via a single bond to a substituted molecule. When a substituent is monovalent, such as, for example, F or Cl, it is bonded to the atom it is substituting by a single bond.

The term “neutral lipid” refers to any of a number of lipid species that exist either in an uncharged or neutral zwitterionic form at a selected pH. At physiological pH, such lipids include, for example, diacylphosphatidylcholine, diacylphosphatidylethanolamine, ceramide, sphingomyelin, cephalin, cholesterol, cerebrosides, and diacylglycerols.

The term “non-cationic lipid” refers to any amphipathic lipid as well as any other neutral lipid or anionic lipid.

The term “nucleic acid” as used herein refers to a polymer containing at least two deoxyribonucleotides or ribonucleotides in either single- or double-stranded form and includes DNA and RNA. DNA may be in the form of, e.g., antisense molecules, plasmid DNA, pre-condensed DNA, a PCR product, vectors (Pl, PAC, BAC, YAC, artificial chromosomes), expression cassettes, chimeric sequences, chromosomal DNA, or derivatives and combinations of these groups. RNA may be in the form of siRNA, asymmetrical interfering RNA (aiRNA), microRNA (miRNA), mRNA, tRNA, rRNA, tRNA, viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2′-O-methyl ribonucleotides, and peptide-nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mal. Cell. Probes, 8:91-98 (1994)).

As used herein, the term “nucleic acid” includes any oligonucleotide or polynucleotide, with fragments containing up to 60 nucleotides generally termed oligonucleotides, and longer fragments termed polynucleotides. In particular embodiments, oligonucleotides of the disclosure are from about 15 to about 60 nucleotides in length.

Nucleic acid may be administered alone in the lipid particles of the disclosure, or in combination (e.g., co-administered) with lipid particles of the disclosure comprising peptides, polypeptides, or small molecules such as conventional drugs. In other embodiments, the nucleic acid may be administered in a viral vector.

“Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkyl halides.

Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al, Nucleic Acid Res., 19:5081 (1991); Ohtsuka et al., J. Biol. Chem., 260:2605-2608 (1985); Rossolini et al., Mol. Cell. Probes, 8:91-98 (1994)).

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such compounds are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985, Easton, PA), which is incorporated herein by reference.

The terms “peptide,” “polypeptide,” and “protein” are used interchangeably herein, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-s- DMG), DSPE-PEG-DBCO, DOPE-PEG-Azide, DSPE-PEG-Azide, DPPE-PEG-Azide, DSPE-PEG-Carboxy-NHS, DOPE-PEG-Carboxylic Acid, DSPE-PEG-Carboxylic acid and the like.

The term “room temperature” as used herein refers to a temperature of about 15 to 28° C.

The term “solvent” as used herein refers to a liquid that can dissolve a solid, liquid, or gas. Non-limiting examples of solvents are silicones, organic compounds, water, alcohols, ionic liquids, and supercritical fluids.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F. C1, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀) hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

A “therapeutic” treatment is a treatment administered to a subject who exhibits signs of pathology, for the purpose of diminishing or eliminating those signs.

The term “therapeutic protein” as used herein refers to a protein or peptide which has a positive or advantageous effect on a condition or disease state of a subject when provided to the subject in a therapeutically effective amount. In certain embodiments, a therapeutic protein or peptide has curative or palliative properties and may be administered to ameliorate, relieve, alleviate, reverse, delay onset of or lessen the severity of one or more symptoms of a disease or disorder. A therapeutic protein or peptide may have prophylactic properties and may be used to delay the onset of a disease or to lessen the severity of such disease or pathological condition. The term “therapeutic protein” includes entire proteins or peptides, and can also refer to therapeutically active fragments thereof. It can also include therapeutically active variants of a protein. Exemplary therapeutic proteins include, but are not limited to, an analgesic protein, an anti-inflammatory protein, an anti-proliferative protein, an proapoptotic protein, an anti-angiogenic protein, a cytotoxic protein, a cytostatic protein, a cytokine, a chemokine, a growth factor, a wound healing protein, a pharmaceutical protein, or a pro-drug activating protein. Therapeutic proteins may include growth factors (EGF, TGF-a, TGF-β, TNF, HGF, IGF, and IL-1-8, inter alia) cytokines, paratopes, Fabs (fragments, antigen binding), and antibodies.

The terms “treat,” “treating” and “treatment,” as used herein, means reducing the frequency or severity with which symptoms of a disease or condition are experienced by a subject by virtue of administering an agent or compound to the subject.

### Lipid Compounds

In one aspect, the present disclosure provides an ionizable lipid compound of Formula (I), or a salt, solvate, stereoisomer, or isotopologue thereof:

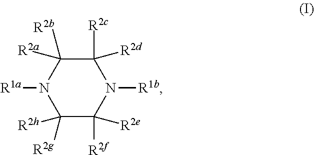

wherein:
R^{1a} and R^{1b} are each independently

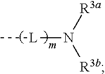

R^{2a}, R^{2b}, R^{2c}, R^{2d}, R^{2e}, R^{2f}, R^{2g}, and R^{2h} are each independently selected from the group consisting of H, optionally substituted C₁-C₁₂ alkyl, optionally substituted C₂-C₁₂ heteroalkyl, optionally substituted C₃-C₈ cycloalkyl, optionally substituted C₂-C₈ heterocycloalkyl, optionally substituted C₂-C₁₂ alkenyl, optionally substituted C₂-C₁₂ alkynyl, optionally substituted C₆-C₁₀ aryl, and optionally substituted C₂-C₁₀ heteroaryl;
each occurrence of R^{3a}, R^{3b}, and R^{3c} is independently selected from the group consisting of H,

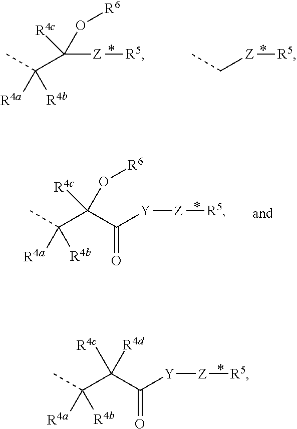

wherein no more than one of R^{3a}, R^{3b}, and R^{3c} is H;
each occurrence of R^{4a}, R^{4b}, R^{4c}, and R^{4d}, if present, is independently selected from the group consisting of optionally substituted C₁-C₁₂ alkyl, halogen, CN, and NO₂;
each occurrence of R⁵ is independently selected from the group consisting of optionally substituted C₁-C₃ alkyl, optionally substituted C₃-C₁₂ cycloalkyl, optionally substituted C₆-C₁₀ aryl, and optionally substituted C₂-C₁₀ heteroaryl;
each occurrence of R⁶ is independently selected from the group consisting of H, optionally substituted C₁-C₆ alkyl, optionally substituted C₃-C₁₂ cycloalkyl, C₂-C₁₂ heterocycloalkyl, optionally substituted C₆-C₁₂ aralkyl, optionally substituted C₆-C₁₂ aryl, optionally substituted C₂-C₁₂ heteroaryl, C(═O)R^{a}, C(═O)OR^{a}, and C(═O)N(R^{a})(R);
each occurrence of L is independently selected from the group consisting of a bond, -(optionally substituted C₁-C₁₂ alkylenyl)-X—, -(optionally substituted C₂-C₁₂ alkenylenyl)-X—, -(optionally substituted C₁-C₁₂ alkynylenyl)-X—, -(optionally substituted C₁-C₁₂ heteroalkylenyl)-X—, optionally substituted C₃-C₈ cycloalkylenyl, and optionally substituted C₂-C₈ heterocyloalkylenyl;
each occurrence of X, if present, is independently selected from the group consisting of a bond, —N(R^{3c})—, and —O—;
each occurrence of Y, if present, is independently selected from the group consisting of a bond, —N(R^{a})—, and —O—;
each occurrence of Z is C₁-C₂₄ alkylenyl,
   wherein the C₁-C₂₄ alkylenyl in each occurrence of Z is independently substituted with at least one substituent selected from the group consisting of C₁-C₁₂ alkyl and C₁-C₁₂ haloalkyl, and
   wherein the C₁-C₂₄ alkylenyl in each occurrence of Z is independently optionally further substituted;
each occurrence of R^{a} and R^{b} is independently selected from the group consisting of H, optionally substituted C₁-C₆ alkyl, optionally substituted C₃-C₈ cycloalkyl, optionally substituted C₁-C₆ haloalkyl, optionally substituted C₆-C₁₂ aralkyl, optionally substituted phenyl, optionally substituted C₂-C₈ heteroaryl, C(═O)R^{c}, C(═O)OR^{c}, and C(═O)N(R^{c})(R^{d});
each occurrence of R^{c} and R^{d} is independently selected from the group consisting of H, optionally substituted C₁-C₆ alkyl, optionally substituted C₃-C₈ cycloalkyl, optionally substituted C₁-C₆ haloalkyl, optionally substituted C₆-C₁₂ aralkyl, optionally substituted phenyl, and optionally substituted C₂-C₈ heteroaryl; and
each occurrence of m is independently 1, 2, 3, or 4.

In certain embodiments, at least one selected from the group consisting of R^{2a}, R^{2b}, R^{2c}, R^{2d}, R^{2e}, R^{2f}, R^{2g}, and R^{2h} is H. In certain embodiments, at least two selected from the group consisting of R^{2a}, R^{2b}, R^{2c}, R^{2d}, R^{2e}, R^{2f}, R^{2g}, and R^{2h} are H. In certain embodiments, at least three selected from the group consisting of R^{2a}, R^{2b}, R^{2c}, R^{2d}, R^{2e}, R^{2f}, R^{2g}, and R^{2h} are H. In certain embodiments, at least four selected from the group consisting of R^{2a}, R^{2b}, R^{2c}, R^{2d}, R^{2e}, R^{2f}, R^{2g}, and R^{2h} are H. In certain embodiments, at least five selected from the group consisting of R^{2a}, R^{2b}, R^{2c}, R^{2d}, R^{2e}, R^{2f}, R^{2g}, and R^{2h} are H. In certain embodiments, at least six selected from the group consisting of R^{2a}, R^{2b}, R^{2c}, R^{2d}, R^{2e}, R^{2f}, R^{2g}, and R^{2h} are H. In certain embodiments, at least seven selected from the group consisting of R^{2a}, R^{2b}, R^{2c}, R^{2d}, R^{2e}, R^{2f}, R^{2g}, and R^{2h} are H. In certain embodiments, each of R^{2a}, R^{2b}, R^{2c}, R^{2d}, R^{2e}, R^{2f}, R^{2g}, and R^{2h} are H.

In certain embodiments, L is —(CH₂)₁₋₁₀—. In certain embodiments, L is —(CH₂)₂₋₁₀NR^{3c}. In certain embodiments, L is —(CH₂)₂₋₁₀O—. In certain embodiments, L is —(CH₂)₁₋₃—CH(OR^{a})—(CH₂)₁₋₃—. In certain embodiments, L is piperazinylenyl. In certain embodiments, L is cyclohexylenyl.

In certain embodiments, L is —CH₂—. In certain embodiments, L is —(CH₂)₂—. In certain embodiments, L is —(CH₂)₃—. In certain embodiments, L is —(CH₂)₁₀—. In certain embodiments, L is —(CH₂)₂O—. In certain embodiments, L is —(CH₂)₃O—. In certain embodiments, L is —CH₂CH(OR^{a})CH₂—. In certain embodiments, L is —(CH₂)₂NR^{3c}—. In certain embodiments, L is

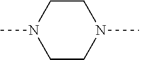

In certain embodiments, L is

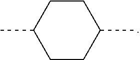

In certain embodiments, L is

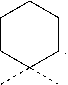

In certain embodiments, the compound of Formula (I) is:

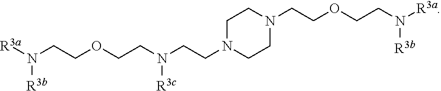

In certain embodiments, the compound of Formula (I) is:

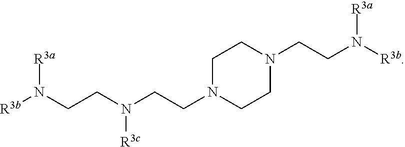

In certain embodiments, the compound of Formula (I) is:

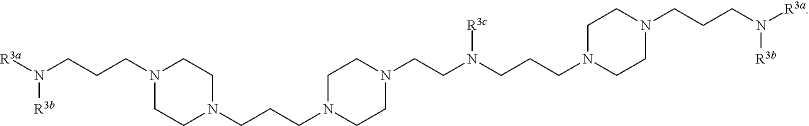

In certain embodiments, the compound of Formula (I) is:

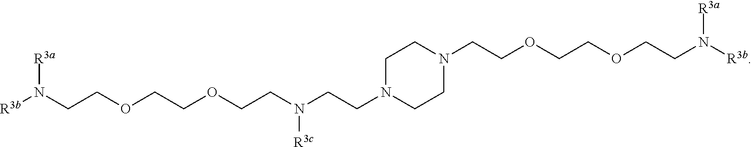

In certain embodiments, the compound of Formula (I) is:

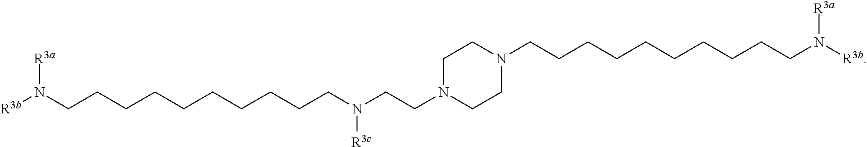

In certain embodiments, the compound of Formula (I) is:

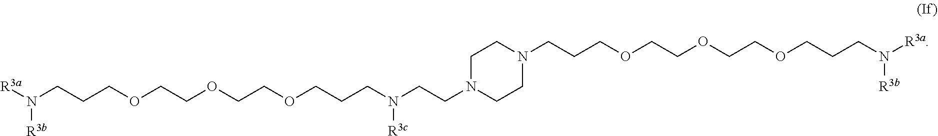

In certain embodiments, the compound of Formula (I) is:

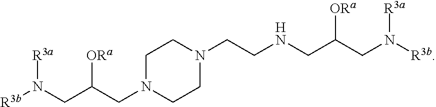

In certain embodiments, the compound of Formula (I) is:

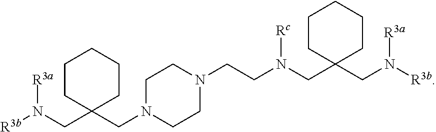

In certain embodiments, the compound of Formula (I) is:

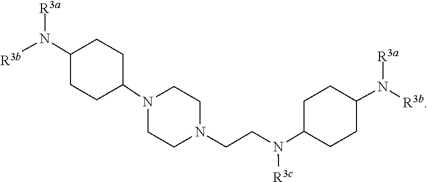

In certain embodiments, R^{4a} is H. In certain embodiments, R^{4b} is H. In certain embodiments, R^{4c} is H. In certain embodiments, R^{4d} is H.

In certain embodiments, R⁵ is methyl.

In certain embodiments, R⁶ is H.

In certain embodiments, each occurrence of Z is independently:

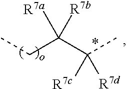

wherein:
each occurrence of R^{7a}, R^{7b}, R^{7c}, and R^{7d} is independently selected from the group consisting of H, C₁-C₆ alkyl, and C₁-C₆ haloalkyl,
   wherein at least one of R^{7a}, R^{7b}, R^{7c}, and R^{7d} is not H; and
each occurrence of o is an integer selected from the group consisting of 1, 2, 3, 4, 5, 6, 7, 8, 9, 10, 11, and 12.

In certain embodiments, R^{7a} is H. In certain embodiments, R^{7b} is H. In certain embodiments, R^{7c} is H. In certain embodiments, R^{7d} is H. In certain embodiments, R^{7a} is methyl. In certain embodiments, R^{7b} is methyl. In certain embodiments, R^{7c} is methyl. In certain embodiments, R^{7d} is methyl.

In certain embodiments, Z is —(CH₂)₄₋₁₀—CH(CH₃)—*. In certain embodiments, Z is —(CH₂)₄₋₁₀—C(CH₃)₂—*. In certain embodiments, Z is —(CH₂)₄₋₁₀—CH(CH₃)—CH₂—*.

In certain embodiments, R^{3a},

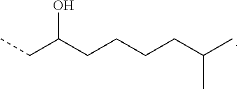

In certain embodiments, R^{3a}, is

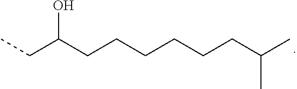

In certain embodiments, R^{3a}, is

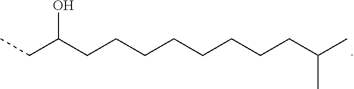

In certain embodiments, R^{3a}, is

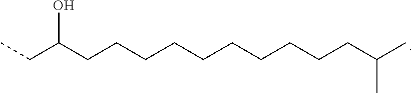

In certain embodiments, R^{3a}, is

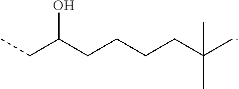

In certain embodiments, R^{3a}, is

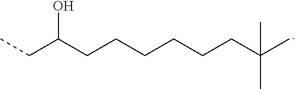

In certain embodiments, R^{3a}, is

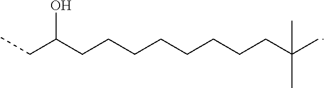

In certain embodiments, R³, is

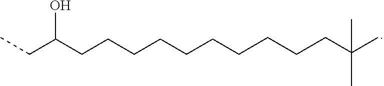

In certain embodiments, R^{3a}, is

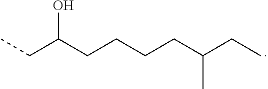

In certain embodiments, R^{3a}, is

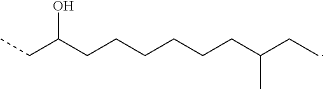

In certain embodiments, R^{3a}, is

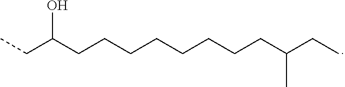

In certain embodiments, R^{3a}, is

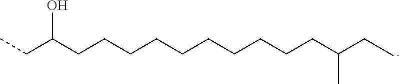

In certain embodiments, R^{3b}, is

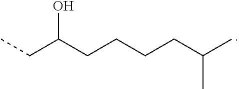

In certain embodiments, R^{3b}, is

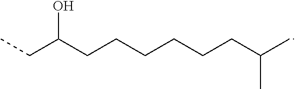

In certain embodiments, R^{3b}, is

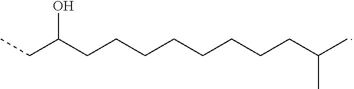

In certain embodiments, R^{3b}, is

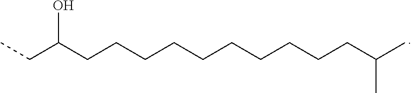

In certain embodiments, R^{3b}, is

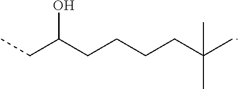

In certain embodiments, R^{3b}, is

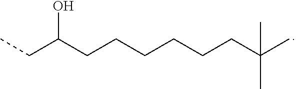

In certain embodiments, R^{3b}, is

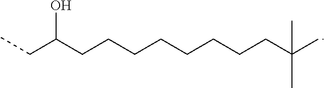

In certain embodiments, R^{3b}, is

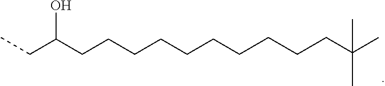

In certain embodiments, R^{3b}, is

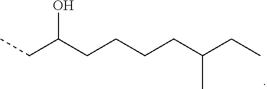

n certain embodiments, R^{3b}, is

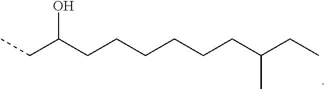

In certain embodiments, R^{3b}, is

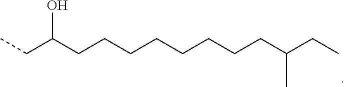

In certain embodiments, R^{3b}, is

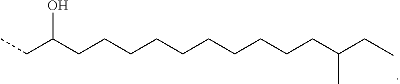

In certain embodiments, R^{3c}, is

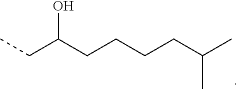

In certain embodiments, R^{3c}, is

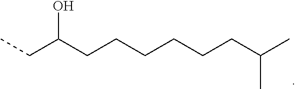

In certain embodiments, R^{3c}, is

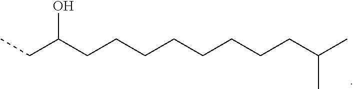

In certain embodiments, R^{3c}, is

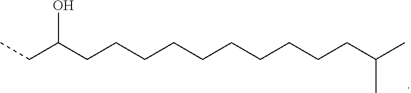

In certain embodiments, R^{3c}, is

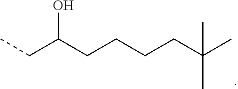

In certain embodiments, R^{3c}, is

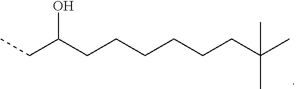

In certain embodiments, R^{3c}, is

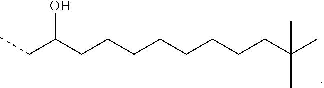

In certain embodiments, R^{3c}, is

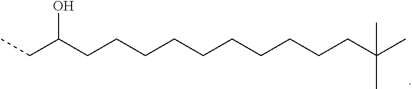

In certain embodiments, R^{3c}, is

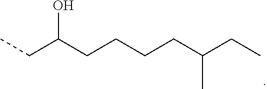

In certain embodiments, R^{3c}, is

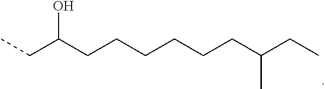

In certain embodiments, R^{3c}, is

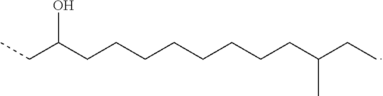

In certain embodiments, R^{3c}, is

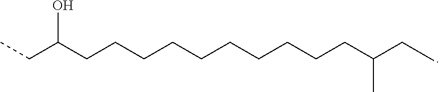

In certain embodiments, each occurrence optionally substituted alkyl, optionally substituted heteroalkyl, optionally substituted cycloalkyl, optionally substituted heterocycloalkyl, optionally substituted alkenyl, optionally substituted alkynyl, optionally substituted aryl, optionally substituted heteroaryl, optionally substituted aralkyl, optionally substituted alkylenyl, optionally substituted alkenylenyl, optionally substituted alkynylenyl, optionally substituted heteroalkylenyl, optionally substituted cycloalkylenyl, and optionally substituted heterocycloalkylenyl, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OH, N(R′)(R″), C(═O)R′, C(═O)OR′, OC(═O)OR′, C(═O)N(R′)(R″), S(═O)₂N(R′)(R″), N(R′)C(═O)R″, N(R′)S(═O)₂R″, C₂-C₈ heteroaryl, and phenyl optionally substituted with at least one halogen, wherein each occurrence of R′ and R″ is independently selected from the group consisting of H, C₁-C₆ alkyl, C₃-C₈ cycloalkyl, C₁-C₆ haloalkyl, benzyl, and phenyl.

In certain embodiments, the compound of Formula (I) is

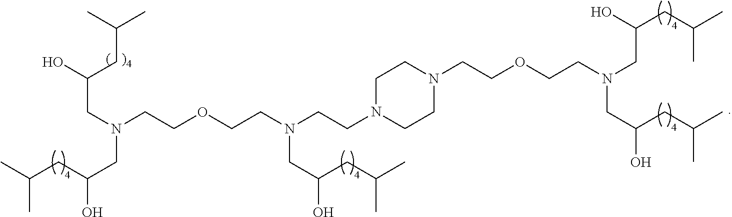

In certain embodiments, the compound of Formula (I) is

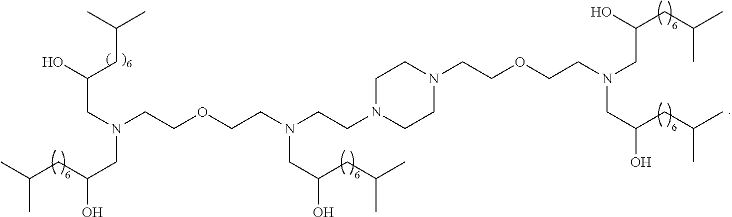

In certain embodiments, the compound of Formula (I) is

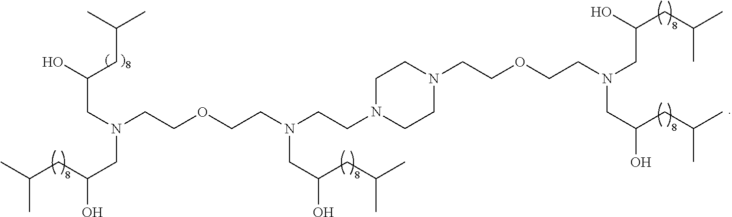

In certain embodiments, the compound of Formula (I) is

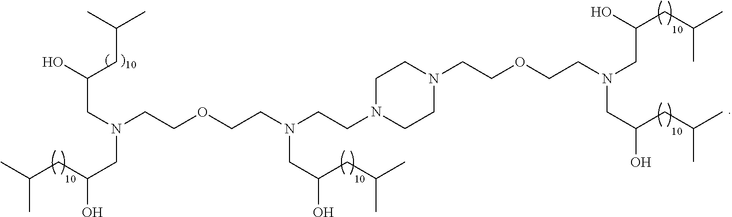

In certain embodiments, the compound of Formula (I) is

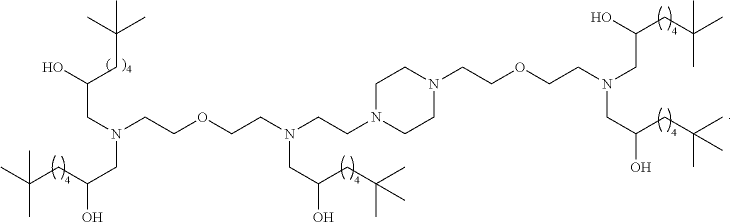

In certain embodiments, the compound of Formula (I) is

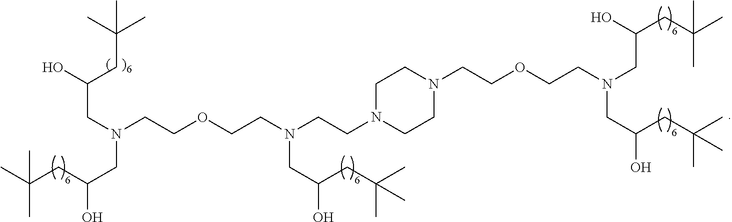

In certain embodiments, the compound of Formula (I) is

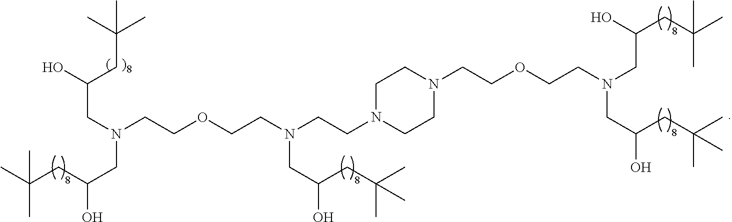

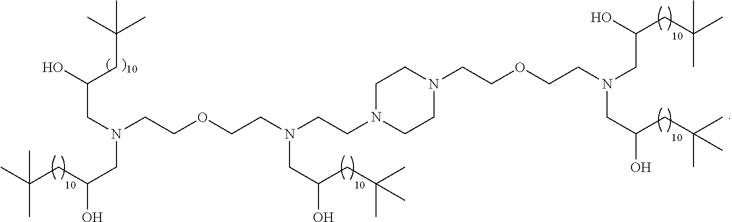

In certain embodiments, the compound of Formula (I) is

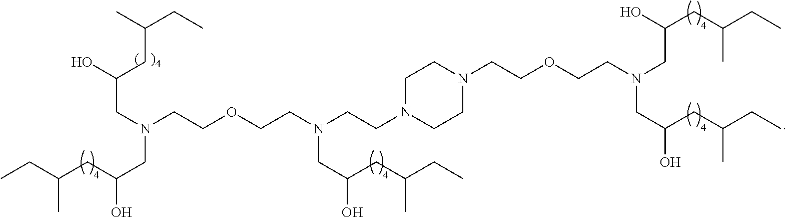

In certain embodiments, the compound of Formula (I) is

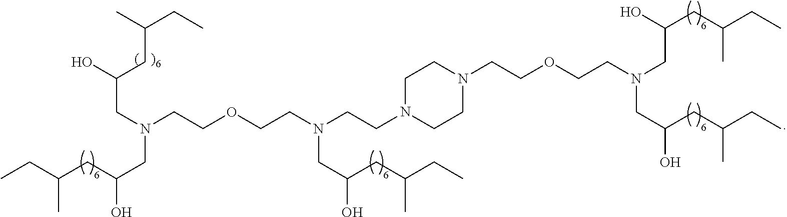

In certain embodiments, the compound of Formula (I) is

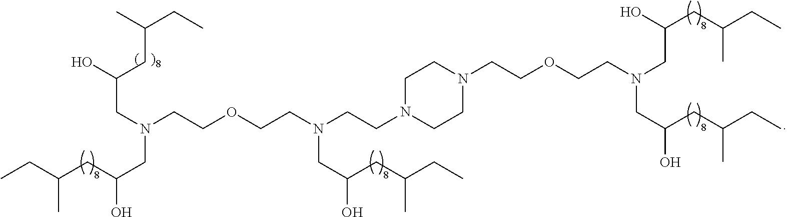

In certain embodiments, the compound of Formula (I) is

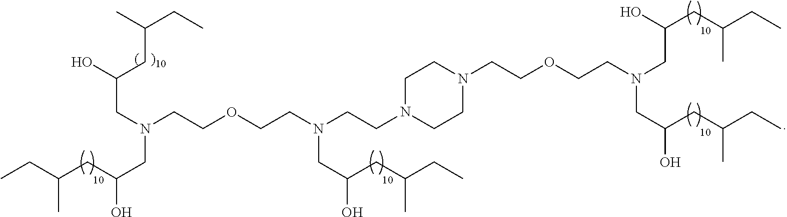

In certain embodiments, the compound of Formula (I) is

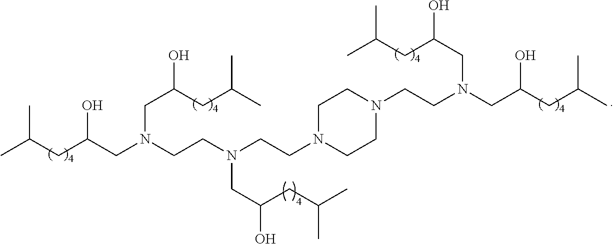

In certain embodiments, the compound of Formula (I) is

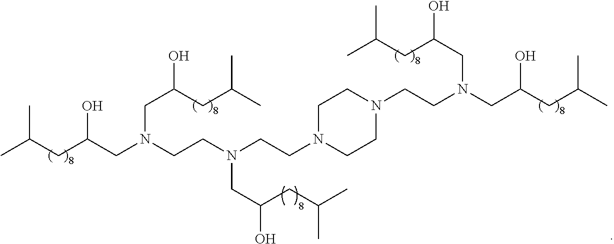

In certain embodiments, the compound of Formula (I) is

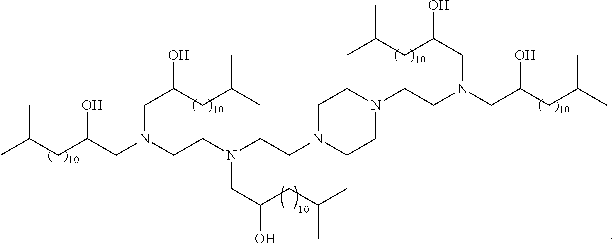

In certain embodiments, the compound of Formula (I) is

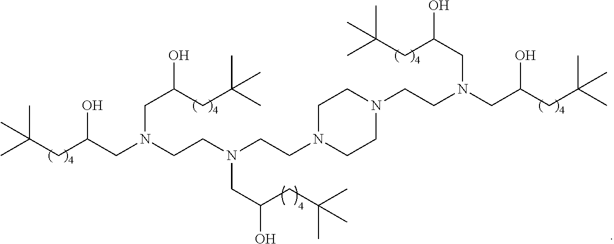

In certain embodiments, the compound of Formula (I) is

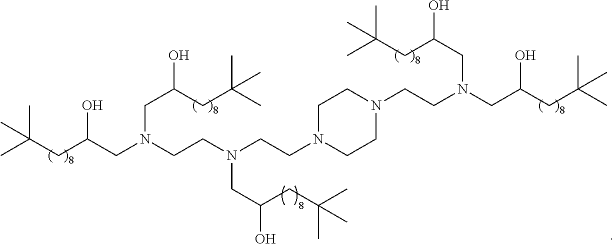

In certain embodiments, the compound of Formula (I) is OH

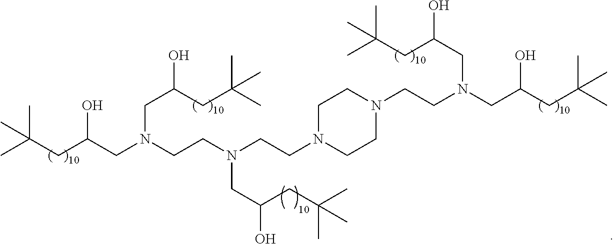

In certain embodiments the compound of Formula (I) is

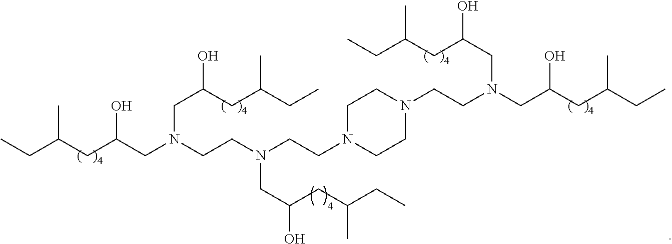

In certain embodiments the compound of Formula (I) is

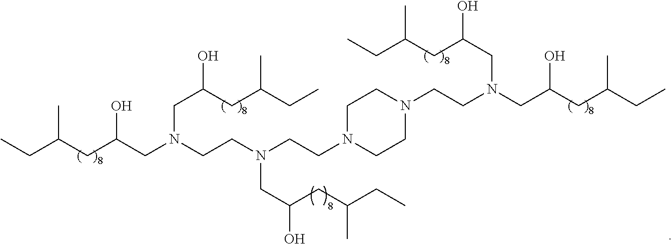

In certain embodiments the compound of Formula (I) is

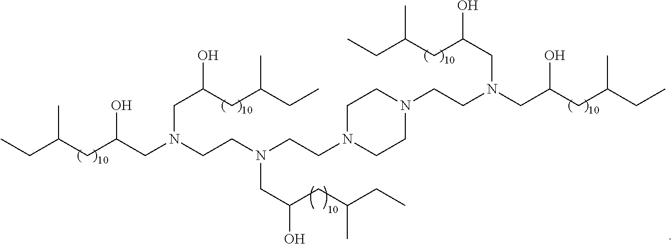

### Ionizable Lipids and or Cationic Lipids

The scope of ionizable lipids contemplated for use in the present disclosure is not limited to ionizable lipids of Formula (I). In the lipid nanoparticles of the disclosure, the cationic lipid or ionizable lipid may comprise, e.g., one or more of the following: (6Z,9Z,28Z,31Z)-heptatriaconta-6,9,28,31-tetraen-19-yl 4-(dimethylamino)butanoate (DLinMC3DMA), [(4-hydroxybutyl)azanediyl]di(hexane-6,1-diyl) bis(2-hexyldecanoate) (ALC-0315), heptadecan-9-yl 8-{(2-hydroxyethyl)[6-oxo-6-(undecyloxy)hexyl]amino}octanoate (SM-102), 1,1′-[[2-[4-[2-[[2-[bis(2-hydroxydodecyl)amino]ethyl](2-hydroxydodecyl)amino]ethyl]-1-piperazinyl]ethyl]imino]bis-2-dodecanol (C12-200), 1,2-dilinoleyloxy-N,N-dimethylaminopropane (DLinDMA), 1,2-dilinolenyloxy-N,N-dimethylaminopropane (DLenDMA), 2,2-dilinoleyl-4-(2-dimethylaminoethyl)-[1,3]-dioxolane (DLin-K-C₂-DMA; “XTC2”), 2,2-dilinoleyl-4-(3-45 dimethylaminopropyl)-1,3]-dioxolane (D Lin-K-C3-D MA), 2,2-dilinoleyl-4-(4-dimethylaminobutyl)-[1,3]-dioxolane (DLin-K-C4-DMA), 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 2,2-dili-noleyl-4-dimethylaminomethyl-[1,3]-dioxolane (DLin-KDMA), 1,2-dilinoleylcarbamoyloxy-3-dimethylaminopropane (D Lin-C-DAP), 1,2-dilinoleyoxy-3-(dimethylaminoacetoxypropane (DLin-DAC), 1-2dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (D Lin-MPZ), 3-(N,N-dilinoleylamino)-1,2-propanediol (D LinAP), 3-(N,N-dioleylamino)-1,2-propanedio (DOAP), 1,2-dilinoleyloxo-3-(2-N,N-dimethylamino)ethoxypropane (D Lin-EG-D MA), N,N-dioleyl-N,N-dimethylammonium chloride (DODAC), 1,2-dioleyloxy-N,N-dimethylaminopropane (DODMA), 1,2-distearyloxy-N,N-dimethylaminopropane (DSD MA), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy)propyl)-N,N, N-trimethylammonium chloride (DOTAP), 3-(N—(N′,N′dimethylaminoethane)-carbamoyl)cholesterol (DC-Chol), N-(1,2-dimyristyloxyprop-3-yl)-N,N-dimethyl-N-hydroxyethyl anrmonium bromide (DMRIE), 2,3-dioleyloxy-N-[2 (spermine-carboxamidoethyl]-N,N-dimethyl-1-propanaminiumtrifluoroacetate (DOSPA), dioctadecylamidoglycyl spermine (DOGS), 3-dimethylamino-2-(cholest-5-en-3-beta-oxvbutan-4-oxy)-1-(cis,cis-9,12-octadecadienoxy)propane (CLinDMA), 2-[5′-(cholest-5-en-3-beta-oxy)-3′-oxapentoxy)-3-dimethyl-1-(cis,cis-9′,1-2′-octadecadienoxy) propane (CpLinDMA), N,N-dimethyl-3,4-dioleyloxybenzylamine (DMOBA), 1,2-N,N′dioleylcarbamyl-3-dimethylaminopropane (DOcarbDAP), 1,2-N,N′-dilinoleylcarbamyl-3-dimethylaminopropane (DLincarbDAP), or mixtures thereof. In certain embodiments, the cationic lipid is DLinDMA, DLin-K-C2-DMA (“XTC2”), or mixtures thereof. The ionizable lipids are not limited to those recited herein, and can further include ionizable lipids known to those skilled in the art, or described in PCT Application No. PCT/US2020/056255 and/or PCT Application No. PCT/US2020/056252, the disclosures of which are herein incorporated by reference in its entirety.

The synthesis of cationic lipids such as DLin-K-C2-DMA (“XTC2”), DLin-K-C3-DMA, DLin-K-C4-DMA, DLin-K6-DMA, and DLin-K-MPZ, as well as additional cationic lipids, is described in U.S. Application Publication No. US 2011/0256175, the disclosure of which is herein incorporated by reference in its entirety for all purposes. The synthesis of cationic lipids such as DLin-K-DMA, DLin-CDAP, DLin-DAC, DLin-MA, DLinDAP, DLin-S-DMA, DLin-2-DMAP, DLin-TMA.Cl, DLin-TAP.Cl, DLin-MPZ, DLinAP, DOAP, and DLin-EG-DMA, as well as additional cationic lipids, is described in PCT Application No. PCT/US08/88676, filed Dec. 31, 2008, the disclosure of which is herein incorporated by reference in its entirety for all purposes. The synthesis of cationic lipids such as CLinDMA, as well as additional cationic lipids, is described in U.S. Patent Publication No. 20060240554, the disclosure of which is herein incorporated by reference in its entirety for all purposes.

### Non-Cationic Lipid

In the nucleic acid-lipid particles of the present disclosure, the non-cationic lipid may comprise, e.g., one or more anionic lipids and/or neutral lipids. In some embodiments, the non-cationic lipid comprises one of the following neutral lipid components: (1) cholesterol or a derivative thereof (2) a phospholipid; or (3) a mixture of a phospholipid and cholesterol or a derivative thereof.

Examples of cholesterol derivatives include, but are not limited to, cholestanol, cholestanone, cholestenone, coprostanol, cholesteryl-2′-hydroxyethyl ether, cholesteryl-4′-hydroxybutyl ether, and mixtures thereof. The synthesis of cholesteryl-2′-hydroxyethyl ether is known to one skilled in the art and described in U.S. Pat. Nos. 8,058,069, 8,492,359, 8,822,668, 9,364,435, 9,504,651, and 11,141,378, all of which are hereby incorporated herein in their entireties for all purposes.

Non-limiting examples of non-cationic lipids include phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), ioleoylphosphatidylethanolamine (DOPE), palmitoyloleoylphosphatidylcholine (POPC), palmitoyloleoylphosphatidylethanolamine (POPE), palmitoyloleyolphosphatidylglycerol (POPG), dioleoylphosphatidylethanolamine 4-(N-maleimidomethyl)-cyclohexane-1-carboxylate DOPE-mal), dipalmitoylphosphatidylethanolamine (DPPE), dimyristoylphosphatidylethanolamine (DMPE), distearoylphosphatidylethanolamine (DSPE), monomethylphosphatidylethanolamine, dimethylphosphatidylethanolamine, dielaidoylphosphatidylethanolamine (DEPE), stearoyloleoylphosphatidylethanolamine (SOPE), lysophosphatidylcholine, dilinoleoylphosphatidylcholine, and mixtures thereof.

Other diacylphosphatidylcholine and diacylphosphatidylethanolamine phospholipids can also be used. The acyl groups in these lipids can be, for example, acyl groups derived from fatty acids having C₁₀-C₂₄ carbon chains, e.g., lauroyl, myristoyl, palmitoyl, stearoyl, or oleoyl. Additional examples of non-cationic lipids include sterols such as cholesterol and derivatives thereof such as cholestanol, cholestanone, cholestenone, coprostanol, cholesteryl-2′-hydroxyethyl ether, cholesteryl-4′-hydroxybutyl ether, and mixtures thereof. In certain embodiments, the phospholipid is DPPC, DSPC, or mixtures thereof.

### Conjugated Lipid

In the nucleic acid-lipid particles of the present disclosure, the conjugated lipid that inhibits aggregation of particles may comprise, e.g., one or more of the following: a polyethylenegly col (PEG) lipid conjugate, a polyamide (ATTA)-lipid conjugate, a cationic-polymer-lipid conjugates (CPLs), or mixtures thereof. In some embodiments, the nucleic acid-lipid particles comprise either a PEG-lipid conjugate or an ATTA-lipid conjugate.

PEG is a linear, water-soluble polymer of ethylene PEG repeating units with two terminal hydroxyl groups. PEGs are classified by their molecular weights; for example, PEG 2000 has an average molecular weight of about 2,000 daltons, and PEG 5000 has an average molecular weight of about 5,000 daltons. PEGs are commercially available from Sigma Chemical Co. and other companies and include, for example, the following: monomethoxypolyethylene glycol (MePEGOH), monomethoxypolyethylene glycolsuccinate (MePEGS), monomethoxypolyethylene glycolsuccinimidyl succinate (MePEG-S-NHS), monomethoxypolyethylene glycolamine (MePEG-NH₂), monomethoxypolyethylene glycoltresylate (MePEG-TRES), and monomethoxypolyethylene glycolimidazolylcarbonyl (MePEG-IM). Other PEGs such as those described in U.S. Pat. Nos. 6,774,180 and 7,053,150 (e.g., mPEG (20 KDa) amine) are also useful for preparing the PEG-lipid conjugates of the present disclosure. The disclosures of these patents are herein incorporated by reference in their entirety for all purposes. In addition, monomethoxypolyethyleneglycolacetic acid (MePEG-CH₂COOH) is particularly useful for preparing PEG-lipid conjugates including, e.g., PEG-DAA conjugates.

In certain embodiments, the PEG-lipid conjugate or ATTA-lipid conjugate is used together with a CPL. The conjugated lipid that inhibits aggregation of particles may comprise a PEG-lipid including, e.g., a PEG-diacylglycerol (DAG), a PEG dialkyloxypropyl (DAA), a PEG-phospholipid, a PEG-ceramide (Cer), or mixtures thereof. The PEGDAA conjugate may be PEG-dilauryloxypropyl (C₁₂), a PEG-dimyristyloxypropyl (C₁₄), a PEG-dipalmityloxypropyl (C₁₆), a PEG-distearyloxypropyl (Cis), or mixtures thereof.

Additional PEG-lipid conjugates suitable for use in the disclosure include, but are not limited to, mPEG2000-1,2-diO-alkyl-sn3-carbomoylglyceride (PEG-C-DOMG). The synthesis of PEG-C-DOMG is described in PCT Application No. PCT/US08/88676, filed Dec. 31, 2008, the disclosure of which is herein incorporated by reference in its entirety for all purposes. Yet additional PEG-lipid conjugates suitable for use in the disclosure include, without limitation, 1-[8′-(1,2-dimyristoyl-3-propanoxy)-carboxamido-3′,6′-dioxaoctanyl]carbamoyl-methyl-poly(ethylene glycol) (2 KPEG-DMG). The synthesis of 2 KPEG-DMG is described in U.S. Pat. No. 7,404,969, the disclosure of which is herein incorporated by reference in its entirety for all purposes.

The PEG moiety of the PEG-lipid conjugates described herein may comprise an average molecular weight ranging from about 550 daltons to about 10,000 daltons. In certain instances, the PEG moiety has an average molecular weight of from about 750 daltons to about 5,000 daltons (e.g., from about 1,000 daltons to about 5,000 daltons, from about 1,500 daltons to about 3,000 daltons, from about 750 daltons to about 3,000 daltons, from about 750 daltons to about 2,000 daltons, etc.). In some embodiments, the PEG moiety has an average molecular weight of about 2,000 daltons or about 750 daltons.

In addition to the foregoing, it will be readily apparent to those of skill in the art that other hydrophilic polymers can be used in place of PEG. Examples of suitable polymers that can be used in place of PEG include, but are not limited to, polyvinylpyrrolidone, polymethyloxazoline, polyethyloxazoline, polyhydroxypropyl methacrylamide, polymethacrylamide and polydimethylacrylamide, polylactic acid, polyglycolic acid, and derivatized celluloses such as hydroxymethylcellulose or hydroxyethylcellulose.

In addition to the foregoing components, the particles (e.g., LNP) of the present disclosure can further comprise cationic poly(ethylene glycol) (PEG) lipids or CPLs (e.g., Chen et al., Bioconj. Chem., 11:433-437 (2000)). Suitable SPLPs and SPLP-CPLs for use in the present disclosure, and methods of making and using SPLPs and SPLP-CPLs, are disclosed, e.g., in U.S. Pat. No. 6,852,334 and PCT Publication No. WO 00/62813, the disclosures of which are herein incorporated by reference in their entirety for all purposes.

In certain instances, the conjugated lipid that inhibits aggregation of particles (e.g., PEG-lipid conjugate) may comprise from about 0.1 mol % to about 2 mol %, from about 0.5 mol % to about 2 mol %, from about 1 mol % to about 2 mol %, from about 0.6 mol % to about 1.9 mol %, from about 0.7 mol % to about 1.8 mol %, from about 0.8 mol % to about 1.7 mol %, from about 1 mol % to about 1.8 mol %, from about 1.2 mol % to about 1.8 mol %, from about 1.2 mol % to about 1.7 mol %, from about 1.3 mol % to about 1.6 mol %, from about 1.4 mol % to about 1.5 mol %, or about 1, 1.1, 1.2, 1.3, 1.4, 1.5, 1.6, 1.7, 1.8, 1.9, or 2 mol % (or any fraction thereof or range therein) of the total lipid present in the particle.

In the lipid nanoparticles of the present disclosure, the active agent or therapeutic agent may be fully encapsulated within the lipid portion of the particle, thereby protecting the active agent or therapeutic agent from enzymatic degradation. In some embodiments, a nucleic acid-lipid particle comprising a nucleic acid such as a messenger RNA (i.e., mRNA) is fully encapsulated within the lipid portion of the particle, thereby protecting the nucleic acid from nuclease degradation. In certain instances, the nucleic acid in the nucleic acid-lipid particle is not substantially degraded after exposure of the particle to a nuclease at 37° C. for at least about 20, 30, 45, or 60 minutes. In certain other instances, the nucleic acid in the nucleic acid-lipid particle is not substantially degraded after incubation of the particle in serum at 37° C. for at least about 30, 45, or 60 minutes or at least about 2, 3, 4, 5, 6, 7, 8, 9, 10, 12, 14, 16, 18, 20, 22, 24, 26, 28, 30, 32, 34, or 36 hours. In other embodiments, the active agent or therapeutic agent (e.g., nucleic acid such as siRNA) is complexed with the lipid portion of the particle. One of the benefits of the formulations of the present disclosure is that the lipid particle compositions are substantially non-toxic to mammals such as humans.

### Synthesis

The present disclosure further provides methods of preparing the compounds of the present disclosure. Compounds of the present teachings can be prepared in accordance with the procedures outlined here, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field.

It is appreciated that where typical or preferred process conditions (i.e., reaction temperatures, times, mole ratios of reactants, solvents, pressures, and so forth) are given, other process conditions can also be used unless otherwise stated. Optimum reaction conditions can vary with the particular reactants or solvent used, but such conditions can be determined by one skilled in the art by routine optimization procedures. Those skilled in the art of organic synthesis will recognize that the nature and order of the synthetic steps presented can be varied for the purpose of optimizing the formation of the compounds described herein.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatography such as high pressure liquid chromatography (HPLC), gas chromatography (GC), gel-permeation chromatography (GPC), or thin layer chromatography (TLC).

Preparation of the compounds can involve protection and deprotection of various chemical groups. The need for protection and deprotection and the selection of appropriate protecting groups can be readily determined by one skilled in the art. The chemistry of protecting groups can be found, for example, in Greene, et al., Protective Groups in Organic Synthesis, 2d. Ed. (Wiley & Sons, 1991), the entire disclosure of which is incorporated by reference herein for all purposes.

The reactions or the processes described herein can be carried out in suitable solvents that can be readily selected by one skilled in the art of organic synthesis. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

In certain embodiment, the compounds synthesized using the methods described herein may contain one or more chiral carbon atoms, giving rise to two or more isomers. The absolute stereochemistry may be depicted using wedge bonds (bold or parallel lines). In certain embodiments, the product formed in any of the reactions described may be a racemate. If a racemate is formed, the isomers making up the racemate may be separated using any suitable method for chiral resolution known to a person skilled in the art. Suitable methods for chiral resolution include, but are not limited to, supercritical fluid chromatography (SFC), chiral HPCL, crystallization, derivatization, or any combination thereof.

In certain embodiments, separation of the isomers formed in one or more separate reactions may require forming a derivative prior to chiral resolution. A non-limiting example of derivatization is protecting one or more functional groups present in a compound using known protecting groups (such as esters, amides, carbamates, ethers, etc.), followed by separation of the isomers by a suitable method. The desired compound is finally obtained through removal of the protecting group.

The present disclosure provides ionizable lipid compounds of Formula (I) and non-limiting exemplary methods for the preparation thereof. One skilled in the art would recognize that alternate techniques and/or methods may be suitable for the synthesis of a compound of Formula (I).

In certain embodiments, the compounds of the present disclosure can be prepared as provided in Schemes 1-5, wherein X¹, X², and X³ are each independently a halogen, s is an integer ranging from 1 to 24, R′ is an optionally substituted alkyl comprising at least one branched or tertiary carbon (i.e., a carbon atom covalently bound to at least 3 carbon atoms), and L, m, and Y are defined within the scope of the present disclosure.

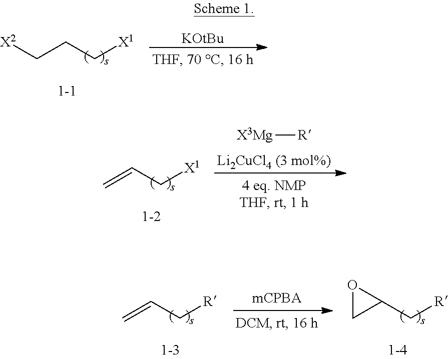

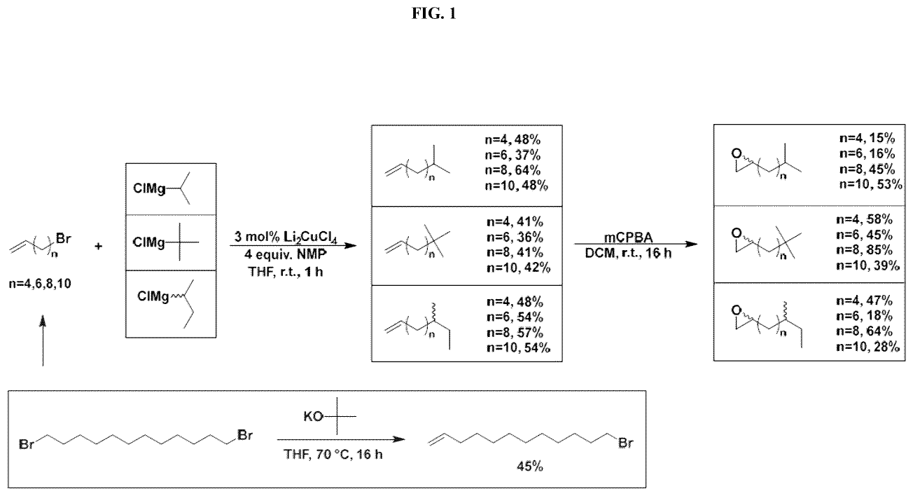

In certain embodiments, homologated haloalkene 1-2 can be prepared from di-haloalkane 1-1 by selective elimination of one halogen in a suitable solvent, including but not limited to tetrahydrofuran (THF), under suitable conditions, including but not limited to a temperature of about 70° C., for a period of time including but not limited to about 16 h. In certain embodiments, alkene 1-3 can be prepared by nucleophilic addition of a Grignard reagent (i.e., R′—MgX³) in the presence of a suitable catalyst, including but not limited to Li₂CuCl₄, in the presence of a polar solvent and/or additive, non-limiting examples including N-methyl pyrrolidone (NMP), further in the presence of a suitable solvent, including but not limited to THF, under suitable reaction conditions, including an ambient reaction temperature, for a period of time including but not limited to 1 h. In certain embodiments, epoxide 1-4 can be prepared from alkene 1-3 by epoxidation with a suitable epoxidation reagent, including but not limited to m-chloroperoxy benzoic acid (mCPBA), under suitable reaction conditions including an ambient reaction temperature, for a period of time including but not limited to 16 h. Exemplary embodiments of the synthetic route depicted in Scheme 1 are provided in FIG. 1.

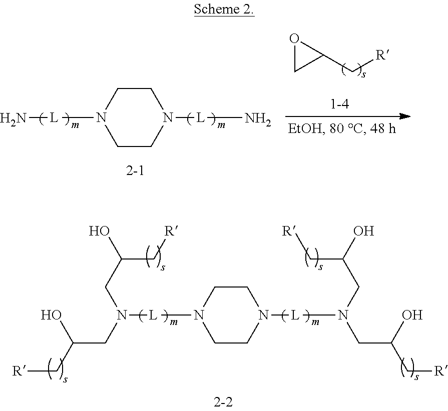

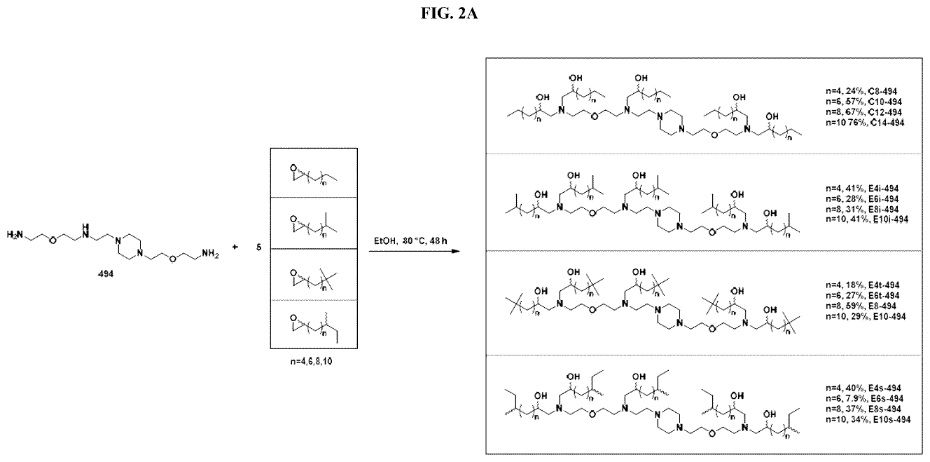

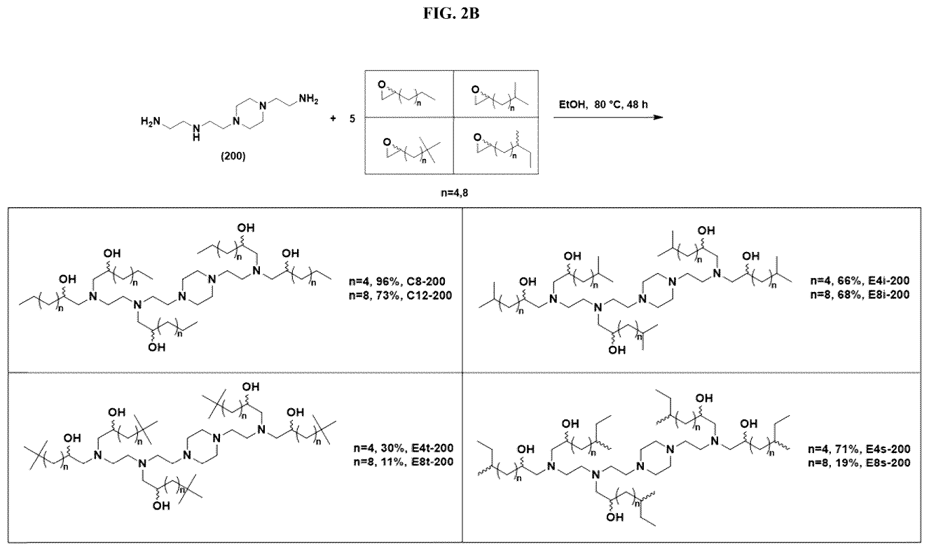

In certain embodiments, polyamine core 2-1 may be alkylated with 4 or more equivalents of epoxide 1-4 to provide ionizable lipid compound 2-2, in the presence of a suitable solvent, including but not limited to ethanol (EtOH), under suitable reaction conditions, including but not limited to a temperature of about 80° C., for a period of time including but not limited to 48 h. In certain embodiments. L comprises a primary or secondary amine. In such embodiments, greater than 4 equivalents of 1-4 are required for full alkylation in embodiments wherein L comprises a primary or secondary amine. In certain embodiments, superstoichiometric quantities of 1-4 may be used to facilitate alkylation. Exemplary embodiments of the synthetic route depicted in Scheme 2 are provided in FIG. 2.

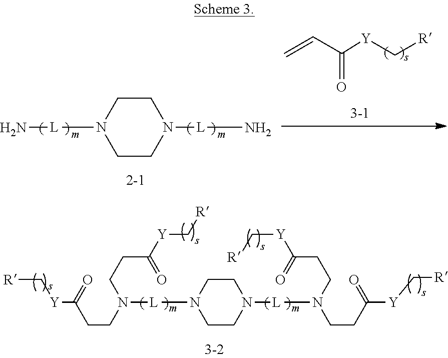

In certain embodiments, polyamine core 2-1 can be conjugated with 4 or more equivalents of α,β-unsaturated carbonyl compound 3-1 (i.e., an α,β-unsaturated ketone, ester, and/or amide) by [1,4]-conjugated addition (i.e., Michael addition reaction) to provide ionizable lipid compound 3-2, in the presence of a suitable solvent, and under suitable reaction conditions.

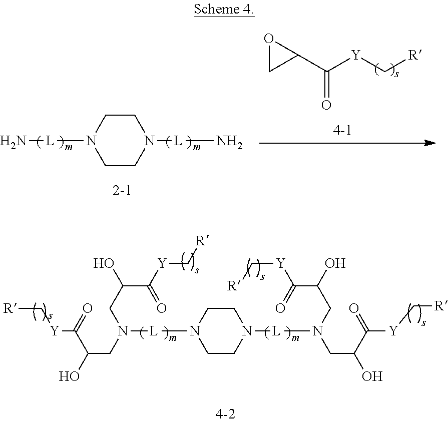

In certain embodiments, polyamine core 2-1 can be conjugated with 4 or more equivalents of α,β-epoxy substituted carbonyl compound 4-1 by nucleophilic addition to provide ionizable lipid compound 4-2, in the presence of a suitable solvent, under suitable reaction conditions.

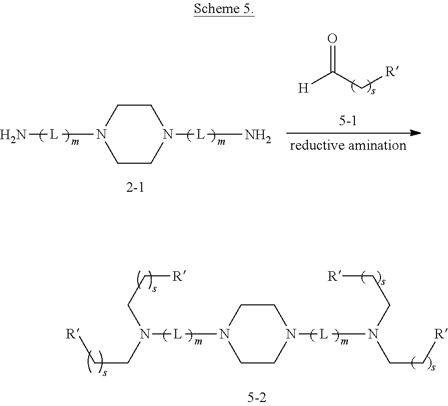

In certain embodiments, polyamine core 2-1 can be conjugated with 4 or more equivalents of aldehyde 5-1 by reductive amination and/or reductive alkylation to provide ionizable lipid compound 5-2, in the presence of a suitable hydride reducing agent, in the presence of a suitable solvent, and under suitable reaction conditions.

Further, the present disclosure exemplifies synthesis of ionizable lipids of Formula (I) (e.g., compounds 2-2, 3-2, 4-2, and 5-2 in Schemes 2-5) wherein polyamine 2-1 is selected from the group consisting of polyamine core 494 (i.e., 2-(2-aminoethoxy)-N-(2-(4-(2-(2-aminoethoxy)ethyl)piperazin-1-yl)ethyl)ethan-1-amine) and polyamine core 200 (i.e., Ni-(2-(4-(2-aminoethyl)piperazin-1-yl)ethyl)ethane-1,2-diamine). The present disclosure is not limited to the embodiments explicitly exemplified herein and alternative polyamine cores are contemplated for use in the present disclosure, including but not limited to 3-(4-(3-aminopropyl)piperazin-1-yl)-N-(2-(4-(3-(4-(3-aminopropyl)piperazin-1-yl)propyl)piperazin-1-yl)ethyl)propan-1-amine, 2-(2-(2-aminoethoxy)ethoxy)-N-(2-(4-(2-(2-(2-aminoethoxy)ethoxy)ethyl)piperazin-1-yl)ethyl)ethan-1-amine, Ni-(2-(4-(10-aminodecyl)piperazin-1-yl)ethyl)decane-1,10-diamine, 3-(2-(2-(3-aminopropoxy)ethoxy)ethoxy)-N-(2-(4-(3-(2-(2-(3-aminopropoxy)ethoxy)ethoxy)propyl)piperazin-1-yl)ethyl)propan-1-amine, Ni-(2-(4-(3-amino-2-ethoxypropyl)piperazin-1-yl)ethyl)-2-ethoxypropane-1,3-diamine, N-((1-(aminomethyl)cyclohexyl)methyl)-2-(4-((1-(aminomethyl)cyclohexyl)methyl)piperazin-1-yl)ethan-1-amine, and N¹-(2-(4-(4-aminocyclohexyl)piperazin-1-yl)ethyl)cyclohexane-1,4-diamine.

TABLE 1
Exemplary ionizable lipids of the present disclosure

| ID | Compound |
|---|---|
| E4i-494 | 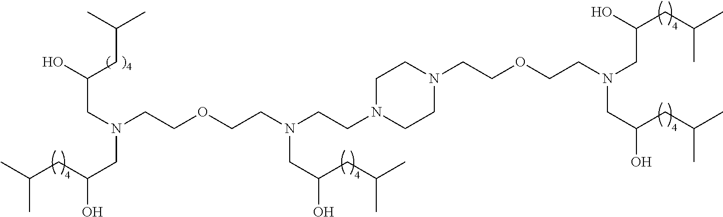 |
| E6i-494 | 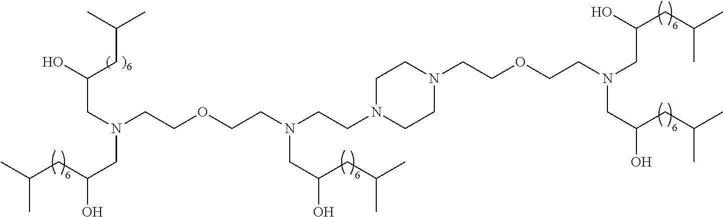 |
| E8i-494 | 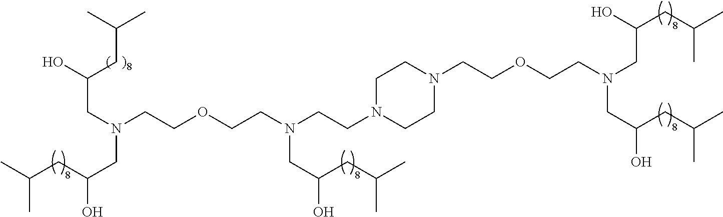 |
| E10i-494 | 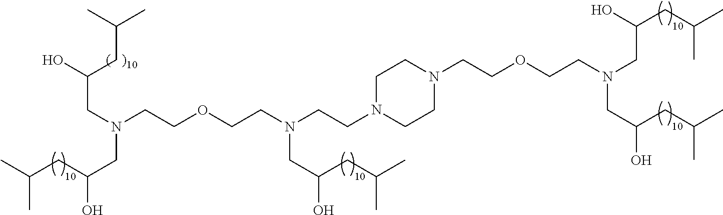 |
| E4t-494 | 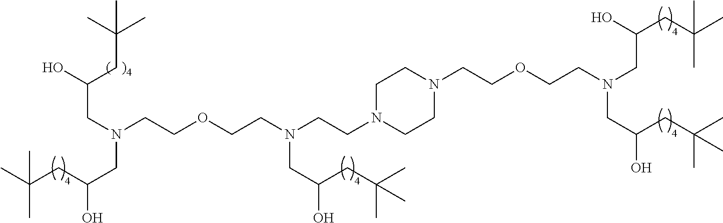 |
| E6t-494 | 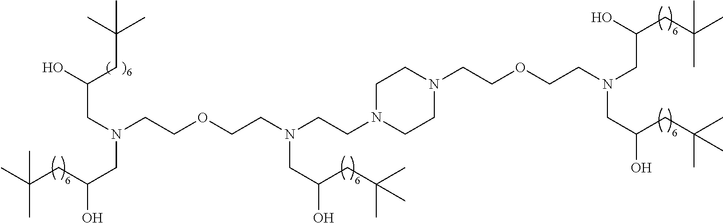 |
| E8t-494 | 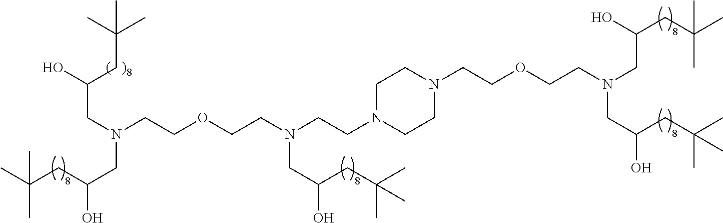 |
| E10t-494 | 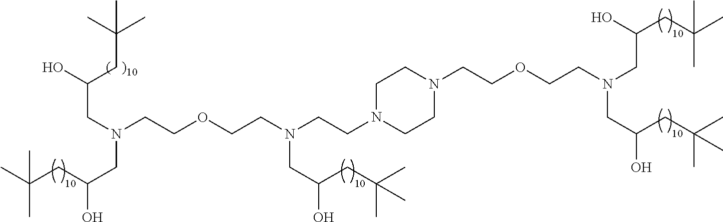 |
| E4s-494 | 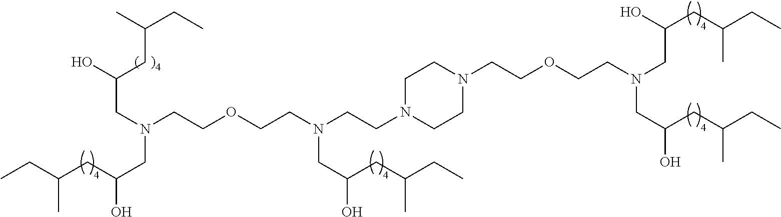 |
| E6s-494 | 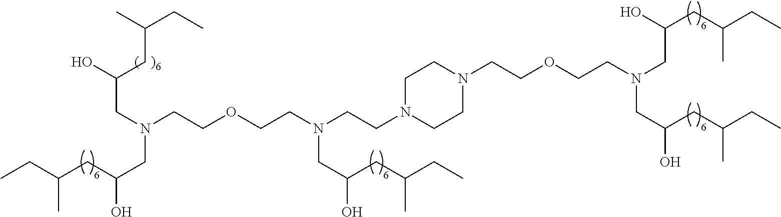 |
| E8s-494 | 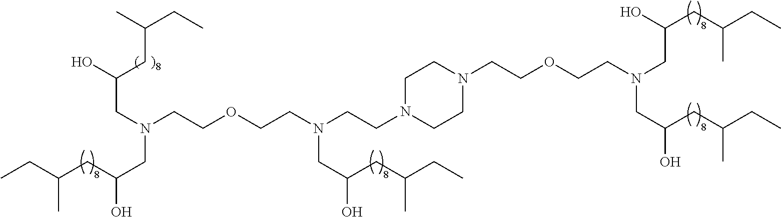 |
| E10s-494 | 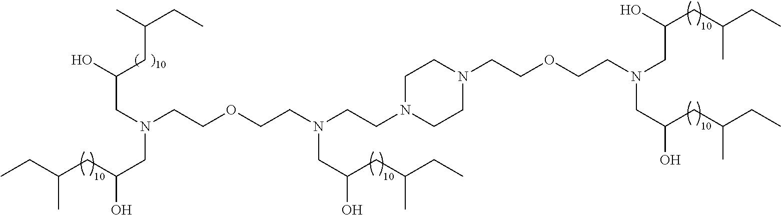 |
| E4i-200 | 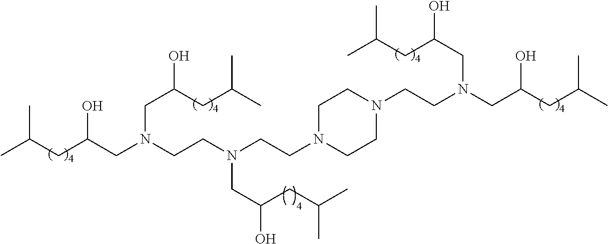 |
| E8i-200 | 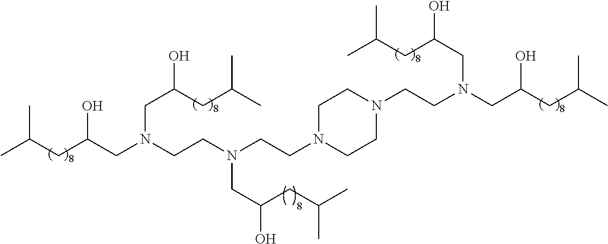 |
| E10i-200 | 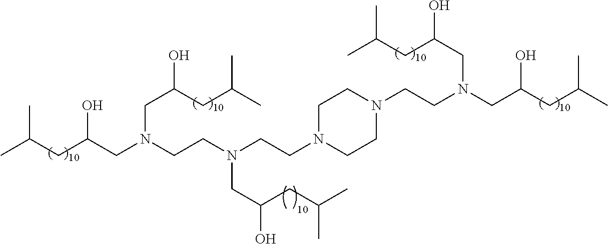 |
| E4t-200 | 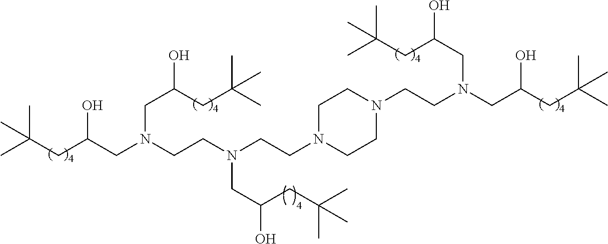 |
| E8t-200 | 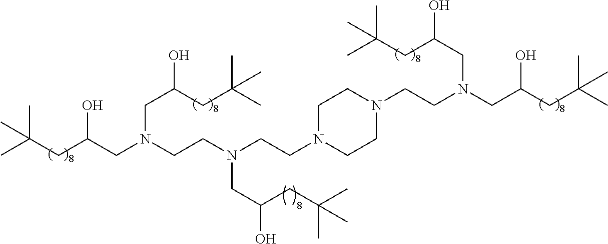 |
| E10t-200 | 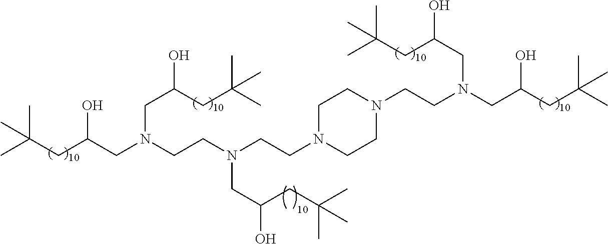 |
| E4s-200 | 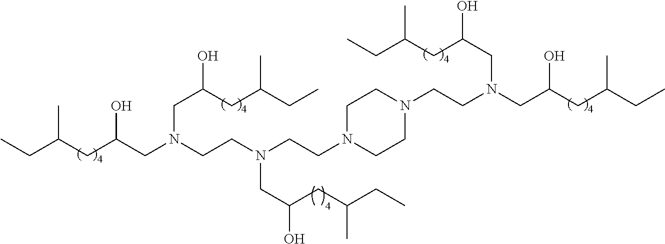 |
| E8s-200 | 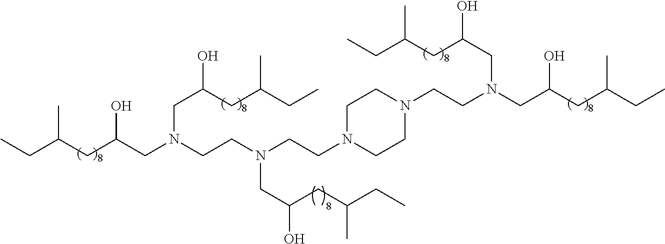 |
| E10s-200 | 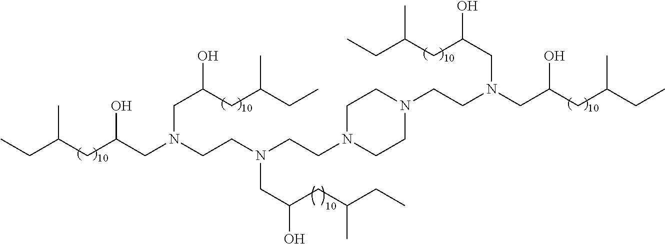 |

### Lipid Nanoparticles (LNPs)

In another aspect, the present disclosure provides a lipid nanoparticle (LNP) composition.

In certain embodiments, the LNP composition comprises at least one ionizable lipid compound or a salt, solvate, stereoisomer, or isotopologue thereof having the structure of Formula (I):

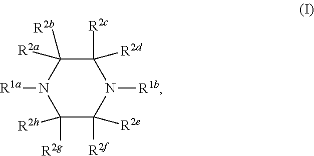

wherein:

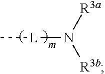

R^{1a} and R^{1b} are each independently R
R^{2a}, R², R^{2c}, R^{2d}, R^{2e}, R^{2f}, R^{2g}, and R^{2h} are each independently selected from the group consisting of H, optionally substituted C₁-C₁₂ alkyl, optionally substituted C₂-C₁₂ heteroalkyl, optionally substituted C₃-C₅ cycloalkyl, optionally substituted C₂-C₈ heterocycloalkyl, optionally substituted C₂-C₁₂ alkenyl, optionally substituted C₂-C₁₂ alkynyl, optionally substituted C₆-C₁₀ aryl, and optionally substituted C₂-C₁₀ heteroaryl;
each occurrence of R^{3a}, R^{3A}, and R³⁰ is independently selected from the group consisting of H,

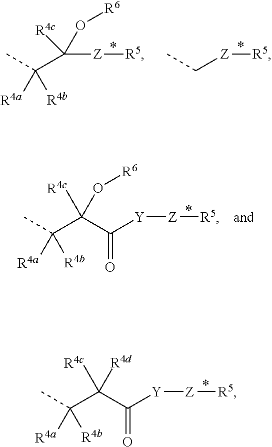

wherein no more than one of R^{3a}, R^{3b}, and R^{3c} is H;
each occurrence of R^{4a}, R^{4b}, R^{4c}, and R^{4d}, if present, is independently selected from the group consisting of optionally substituted C₁-C₁₂ alkyl, halogen, CN, and NO₂;
each occurrence of R⁵ is independently selected from the group consisting of optionally substituted C₁-C₃ alkyl, optionally substituted C₃-C₁₂ cycloalkyl, optionally substituted C₆-C₁₀ aryl, and optionally substituted C₂-C₁₀ heteroaryl;
each occurrence of R⁶ is independently selected from the group consisting of H, optionally substituted C₁-C₆ alkyl, optionally substituted C₃-C₁₂ cycloalkyl, C₂-C₁₂ heterocycloalkyl, optionally substituted C₆-C₁₂ aralkyl, optionally substituted C₆-C₁₂ aryl, optionally substituted C₂-C₁₂ heteroaryl, C(═O)R^{a}, C(═O)OR^{a}, and C(═O)N(R^{a})(R^{b});
each occurrence of L is independently selected from the group consisting of a bond, -(optionally substituted C₁-C₁₂ alkylenyl)-X—, -(optionally substituted C₂-C₁₂ alkenylenyl)-X—, -(optionally substituted C₁-C₁₂ alkynylenyl)-X—, -(optionally substituted C₁-C₁₂ heteroalkylenyl)-X—, optionally substituted C₃-C₈ cycloalkylenyl, and optionally substituted C₂-C₈ heterocyloalkylenyl;
each occurrence of X, if present, is independently selected from the group consisting of a bond, —N(R^{3c})—, and —O—;
each occurrence of Y, if present, is independently selected from the group consisting of a bond, —N(R^{a})—, and —O—;
each occurrence of Z is C₁-C₂₄ alkylenyl,
   wherein the C₁-C₂₄ alkylenyl in each occurrence of Z is independently substituted with at least one substituent selected from the group consisting of C₁-C₁₂ alkyl and C₁-C₁₂ haloalkyl, and
   wherein the C₁-C₂₄ alkylenyl in each occurrence of Z is independently optionally further substituted;
each occurrence of R^{a} and R^{b} is independently selected from the group consisting of H, optionally substituted C₁-C₆ alkyl, optionally substituted C₃-C₈ cycloalkyl, optionally substituted C₁-C₆ haloalkyl, optionally substituted C₆-C₁₂ aralkyl, optionally substituted phenyl, optionally substituted C₂-C₈ heteroaryl, C(═O)R^{c}, C(═O)OR^{c}, and C(═O)N(R)(R^{d});
each occurrence of R^{c} and R^{d} is independently selected from the group consisting of H, optionally substituted C₁-C₆ alkyl, optionally substituted C₃-C₈ cycloalkyl, optionally substituted C₁-C₆ haloalkyl, optionally substituted C₆-C₁₂ aralkyl, optionally substituted phenyl, and optionally substituted C₂-C₈ heteroaryl; and
   each occurrence of m is independently an integer selected from the group consisting of 1, 2, 3, and 4;

In certain embodiments, the LNP composition comprises at least one neutral lipid.

In certain embodiments, the LNP composition comprises cholesterol.

In certain embodiments, the LNP composition comprises at least one conjugated lipid.

In certain embodiments, the LNP comprises at least one nucleic acid and/or therapeutic agent cargo, wherein the cargo is at least partially encapsulated therein.

In certain embodiments, at least one selected from the group consisting of R^{2a}, R^{2b}, R^{2c}, R^{2d}, R^{2e}, R^{2f}, R^{2g}, and R^{2h} is H. In certain embodiments, at least two selected from the group consisting of R^{2a}, R^{2b}, R^{2c}, R^{2d}, R^{2e}, Rf, R^{2g}, and R^{2h} are H. In certain embodiments, at least three selected from the group consisting of R^{2a}, R^{2b}, R^{2c}, R^{2d}, R^{2e}, R^{2f}, R^{2g}, and R^{2h} are H. In certain embodiments, at least four selected from the group consisting of R^{2a}, R^{2b}, R^{2c}, R^{2d}, R^{2e}, R^{2f}, R^{2g}, and R^{2h} are H. In certain embodiments, at least five selected from the group consisting of R^{2a}, R^{2b}, R^{2c}, R^{2d}, R^{2e}, R^{2f}, R^{2g}, and R^{2h} are H. In certain embodiments, at least six selected from the group consisting of R^{2a}, R^{2b}, R^{2c}, R^{2d}, R^{2e}, R^{2f}, R^{2g}, and R^{2h} are H. In certain embodiments, at least seven selected from the group consisting of R^{2a}, R^{2b}, R^{2c}, R^{2d}, R^{2e}, R^{2f}, R^{2g}, and R^{2h} are H. In certain embodiments, each of R^{2a}, R^{2b}, R^{2c}, R^{2d}, R^{2e}, R^{2f}, R^{2g}, and R^{2h} are H.

In certain embodiments, L is —(CH₂)₁₋₁₀—. In certain embodiments, L is —(CH₂)₂₋₁₀NR^{3c}—. In certain embodiments, L is —(CH₂)₂₋₁₀O—. In certain embodiments, L is —(CH₂)₁₋₃—CH(OR^{a})—(CH₂)₁₋₃—. In certain embodiments, L is piperazinylenyl. In certain embodiments, L is cyclohexylenyl.

In certain embodiments, L is —CH₂—. In certain embodiments, L is —(CH₂)₂—. In certain embodiments, L is —(CH₂)₃—. In certain embodiments, L is —(CH₂)₁₀—. In certain embodiments, L is —(CH₂)₂O—. In certain embodiments, L is —(CH₂)₃O—. In certain embodiments, L is —CH₂CH(OR^{a})CH₂—. In certain embodiments, L is —(CH₂)₂NR^{3c}—. In certain embodiments L

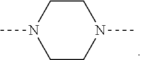

In certain embodiments, L is

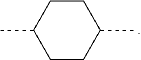

In certain embodiments, L is

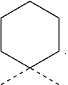

In certain embodiments, the compound of Formula (I) is:

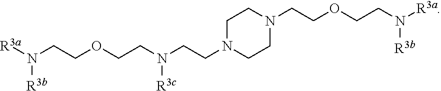

In certain embodiments, the compound of Formula (I) is:

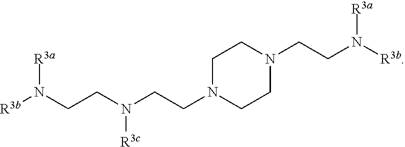

In certain embodiments, the compound of Formula (I) is:

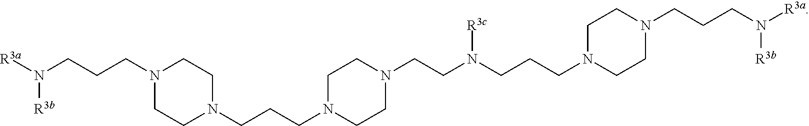

In certain embodiments, the compound of Formula (I) is:

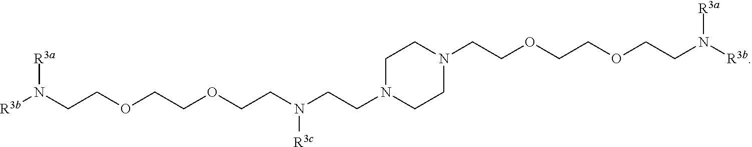

In certain embodiments, the compound of Formula (I) is:

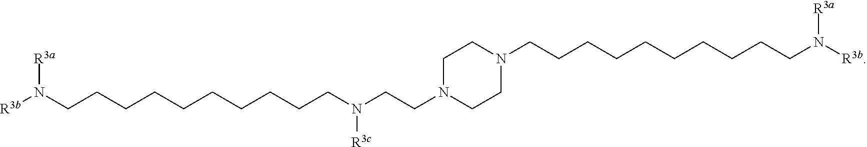

In certain embodiments, the compound of Formula (I) is:

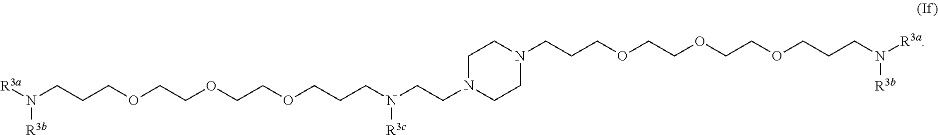

In certain embodiments, the compound of Formula (I) is:

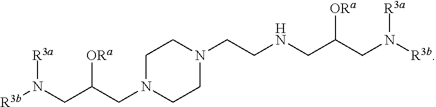

In certain embodiments, the compound of Formula (I) is:

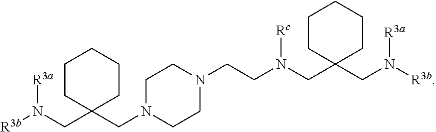

In certain embodiments, the compound of Formula (I) is:

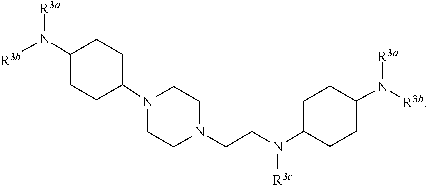

In certain embodiments, R^{4a} is H. In certain embodiments, R^{4b} is H. In certain embodiments, R^{4c} is H. In certain embodiments, R^{4d} is H.

In certain embodiments, R⁵ is methyl.

In certain embodiments, R⁶ is H.

In certain embodiments, each occurrence of Z is independently:

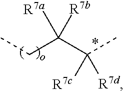

wherein:
each occurrence of R^{7a}, R^{7b}, R^{7c}, and R^{7d} is independently selected from the group consisting of H, C₁-C₆ alkyl, and C₁-C₆ haloalkyl,
   wherein at least one of R^{7a}, R^{7b}, R^{7c}, and R^{7d} is not H; and
each occurrence of o is an integer selected from the group consisting of 1, 2, 3, 4, 5, 6, 7, 8, 9, 10, 11, and 12.

In certain embodiments, R^{7a} is H. In certain embodiments, R^{7b} is H. In certain embodiments, R^{7c} is H. In certain embodiments. R^{7d} is H. In certain embodiments, R^{7a} is methyl. In certain embodiments, R^{7b} is methyl. In certain embodiments, R^{7c} is methyl. In certain embodiments, R^{7d} is methyl.

In certain embodiments, Z is —(CH₂)₄₋₁₀—CH(CH₃)—*. In certain embodiments, Z is —(CH₂)₄₋₁₀—C(CH₃)₂—*. In certain embodiments, Z is —(CH₂)₄₋₁₀—CH(CH₃)—CH₂—*.

In certain embodiments, R^{3a}, is

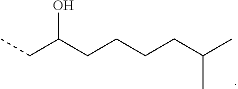

In certain embodiments, R^{3a}, is

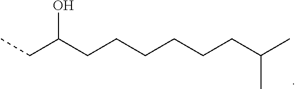

In certain embodiments, R^{3a}, is

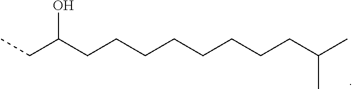

In certain embodiments, R^{3a}, is

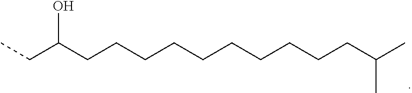

In certain embodiments, R^{3a}, is

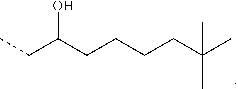

In certain embodiments, R^{3a}, is

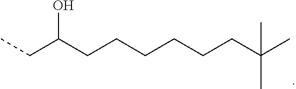

In certain embodiments, R^{3a}, is

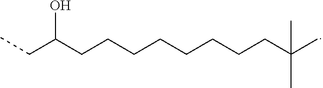

In certain embodiments, R^{3a}, is

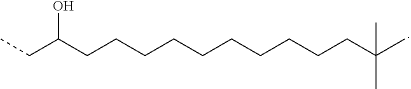

In certain embodiments, R^{3a}, is

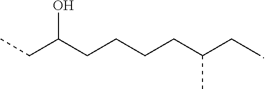

In certain embodiments, R^{3a}, is

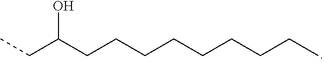

In certain embodiments, R^{3a}, is

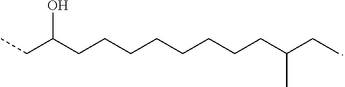

In certain embodiments, R^{3a}, is

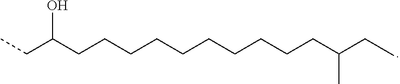

In certain embodiments, R^{3b}, is

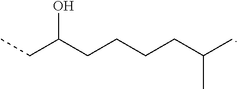

In certain embodiments, R^{3b}, is

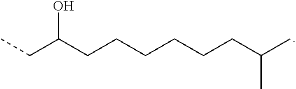

In certain embodiments, R^{3b}, is

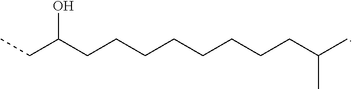

In certain embodiments, R^{3b},

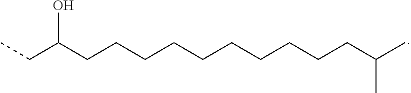

In certain embodiments, R^{3b}, is

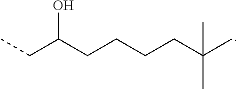

In certain embodiments, R^{3b}, is

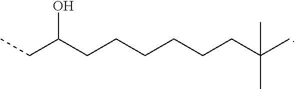

In certain embodiments, R^{3b}, is

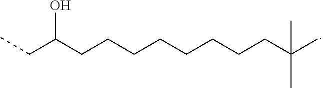

In certain embodiments, R^{3b}, is

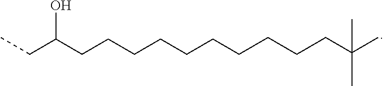

In certain embodiments, R^{3b}, is

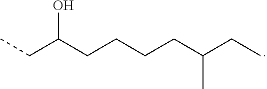

In certain embodiments, R^{3b}, is

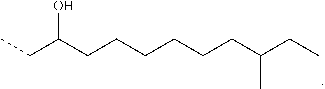

In certain embodiments, R^{3b}, is

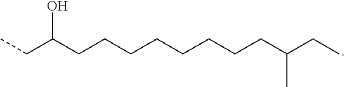

In certain embodiments, R^{3b}, is

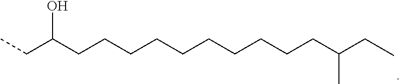

In certain embodiments, R^{3c}, is

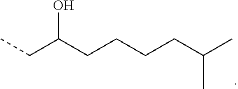

In certain embodiments, R^{3c}, is

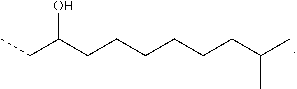

In certain embodiments, R^{3c}, is

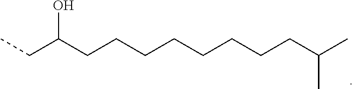

In certain embodiments, R^{3c}, is

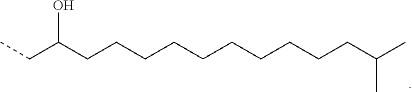

In certain embodiments, R^{3c}, is

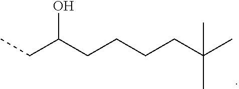

In certain embodiments, R^{3c}, is

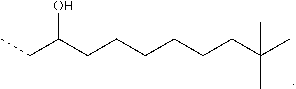

In certain embodiments, R^{3c}, is

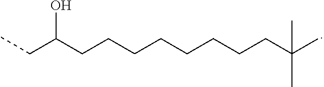

In certain embodiments, R^{3c}, is

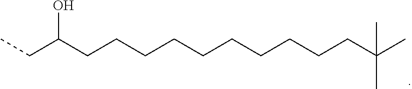

In certain embodiments, R^{3c}, is

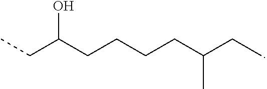

In certain embodiments, R^{3c}, is

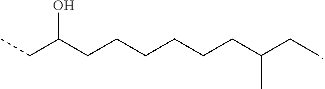

In certain embodiments, R^{3c}, is

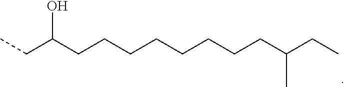

In certain embodiments, R^{3c}, is

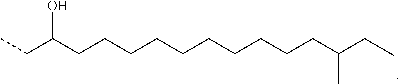

In certain embodiments, each occurrence of optionally substituted alkyl, optionally substituted heteroalkyl, optionally substituted cycloalkyl, optionally substituted heterocycloalkyl, optionally substituted alkenyl, optionally substituted alkynyl, optionally substituted aryl, optionally substituted heteroaryl, optionally substituted aralkyl, optionally substituted alkylenyl, optionally substituted alkenylenyl, optionally substituted alkynylenyl, optionally substituted heteroalkylenyl, optionally substituted cycloalkylenyl, and optionally substituted heterocycloalkylenyl, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OH, N(R′)(R″), C(═O)R′, C(═O)OR′, OC(═O)OR′, C(═O)N(R′)(R″), S(═O)₂N(R′)(R″), N(R′)C(═O)R″, N(R′)S(═O)₂R″, C₂-C₈ heteroaryl, and phenyl optionally substituted with at least one halogen, wherein each occurrence of R′ and R″ is independently selected from the group consisting of H, C₁-C₆ alkyl, C₃-C₈ cycloalkyl, C₁-C₆ haloalkyl, benzyl, and phenyl.

In certain embodiments, the compound of Formula (I) is

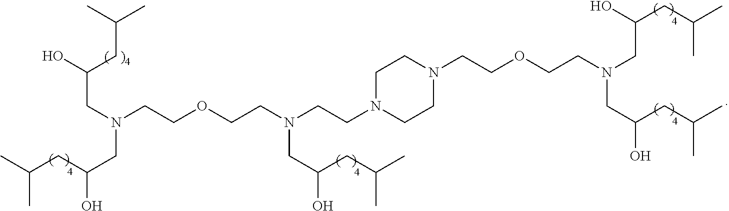

In certain embodiments, the compound of Formula (I) is

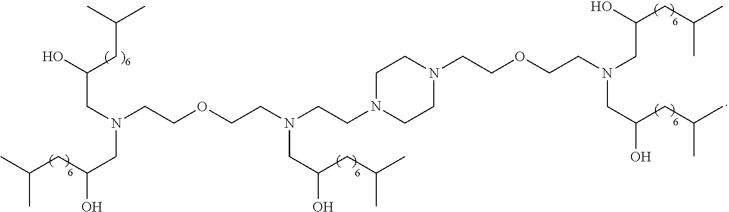

In certain embodiments, the compound of Formula (I) is

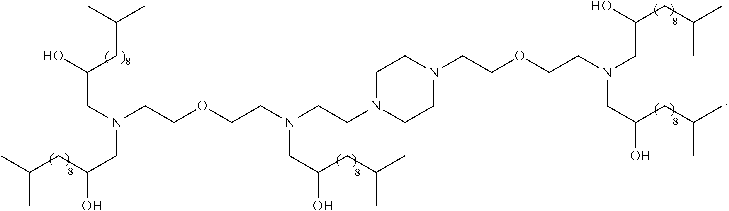

In certain embodiments, the compound of Formula (I) is

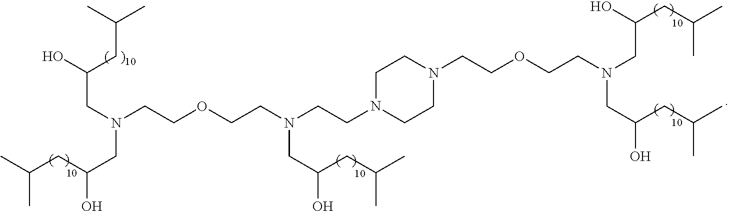

In certain embodiments, the compound of Formula (I) is

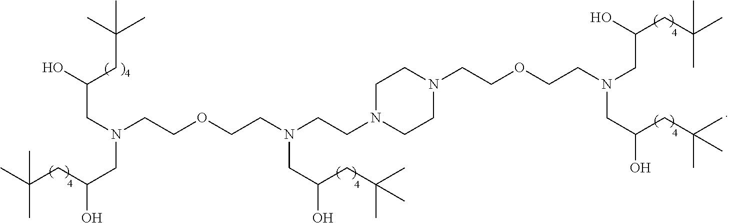

In certain embodiments, the compound of Formula (I) is

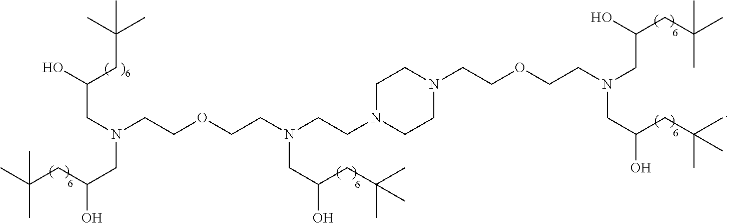

In certain embodiments, the compound of Formula (I) is

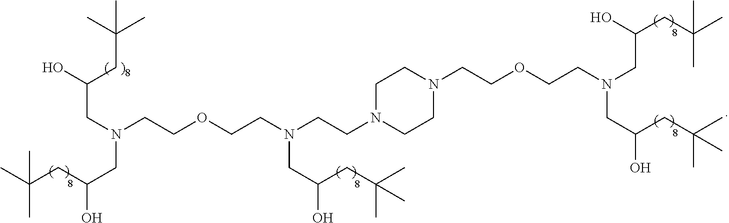

In certain embodiments, the compound of Formula (I) is

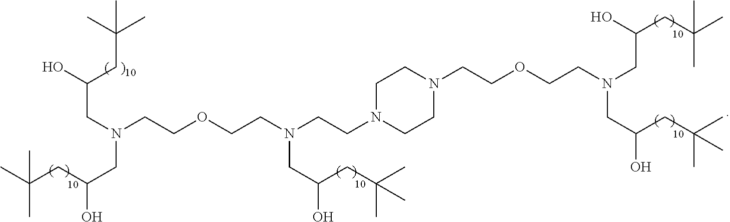

In certain embodiments, the compound of Formula (I) is

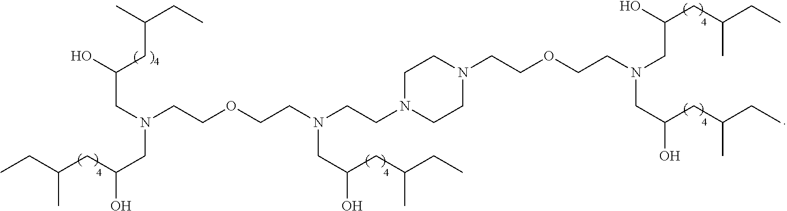

In certain embodiments, the compound of Formula (I) is

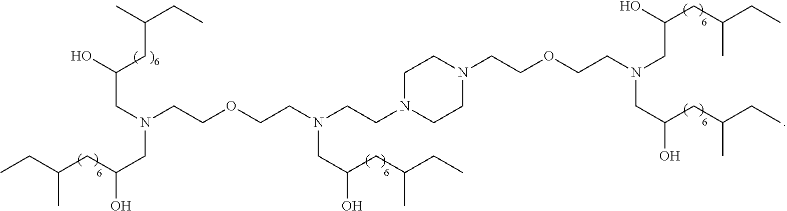

In certain embodiments, the compound of Formula (I) is

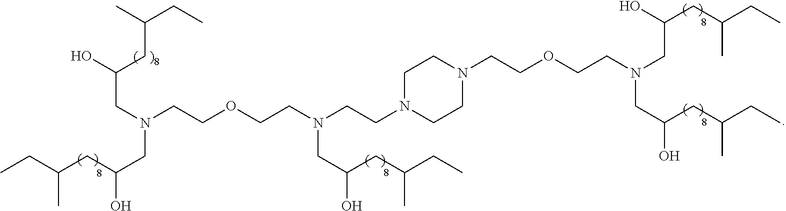

In certain embodiments, the compound of Formula (I) is

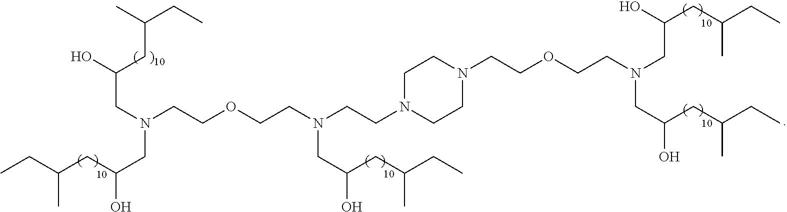

In certain embodiments, the compound of Formula (I) is

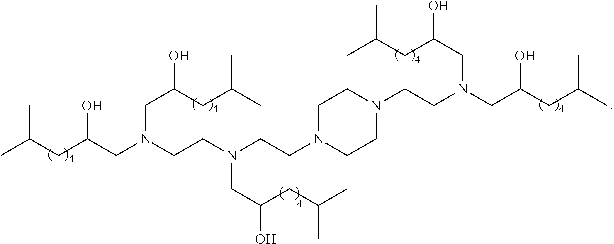

In certain embodiments, the compound of Formula (I) is

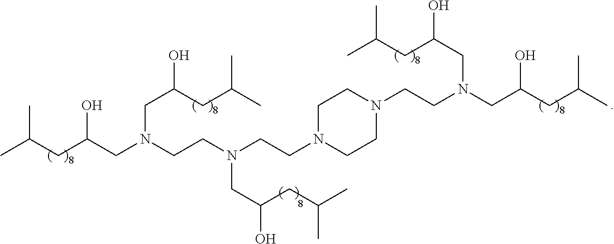

In certain embodiments, the compound of Formula (I) is

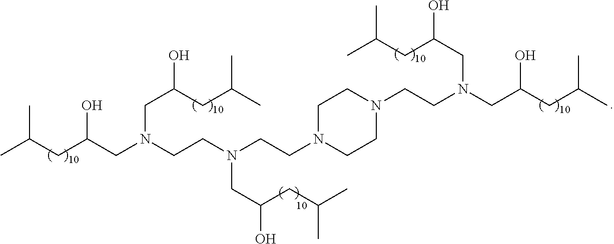

In certain embodiments, the compound of Formula (I) is

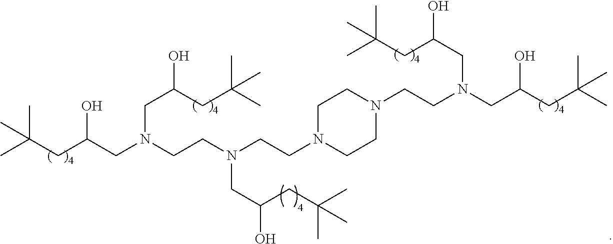

In certain embodiments, the compound of Formula (I) is

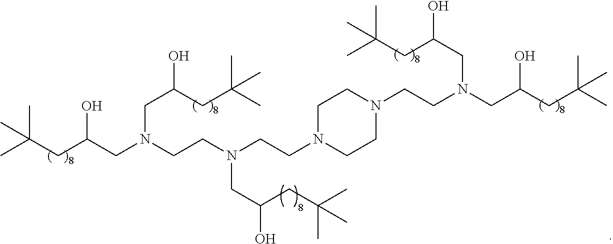

In certain embodiments, the compound of Formula (I) is

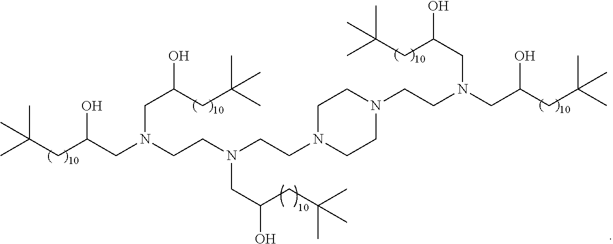

In certain embodiments, the compound of Formula (I) is

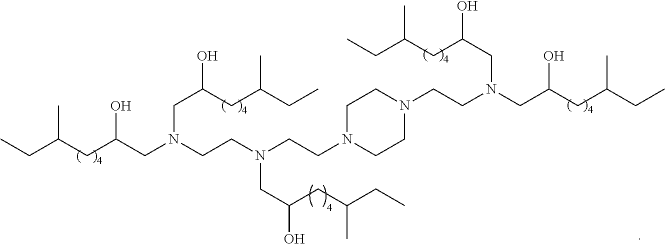

In certain embodiments, the compound of Formula (I) is

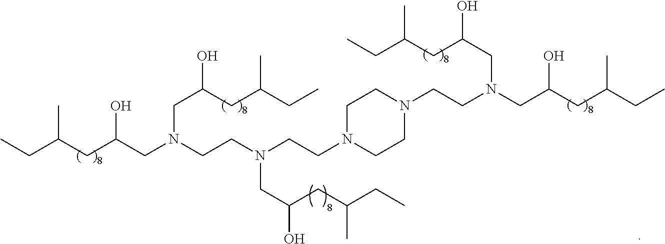

In certain embodiments, the compound of Formula (I) is

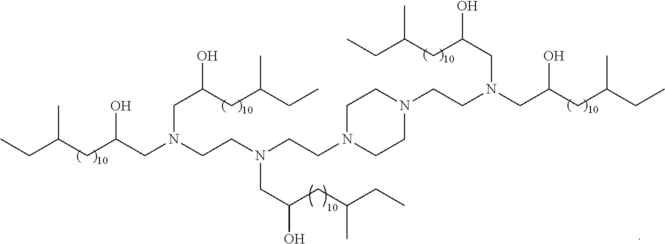

In certain embodiments, the at least one ionizable lipid of Formula (I) comprises about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or about 90 mol % of the LNP.

In certain embodiments, the at least one ionizable lipid of Formula (I) comprises less than about 10, 11, 12, 13, 14, 15, 16, 17, 18, 19, 20, 21, 22, 23, 24, 25,26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or about 90 mol % of the LNP.

In certain embodiments, the at least one ionizable lipid of Formula (I) comprises greater than about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or about 90 mol % of the LNP.

In certain embodiments, the at least one ionizable lipid of Formula (I) comprises about 35 mol % of the LNP. In certain embodiments, the at least one ionizable lipid of Formula (I) comprises less than about 35 mol % of the LNP. In certain embodiments, the at least one ionizable lipid of Formula (I) comprises greater than about 35 mol % of the LNP.

In certain embodiments, the at least one ionizable lipid of Formula (I) comprises about 40 mol % of the LNP. In certain embodiments, the at least one ionizable lipid of Formula (I) comprises less than about 40 mol % of the LNP. In certain embodiments, the at least one ionizable lipid of Formula (I) comprises greater than about 40 mol % of the LNP.

In certain embodiments, the at least one neutral lipid comprises about 1, 2, 3, 4, 5, 6, 7, 8,9, 10, 11, 12, 13, 14, 15, 16, 17, 18, 19, 20, 21,22,23,24,25,26,27,28,29, 30, 31, 32, 33, 34, 35, 36, 37, 38, 39, or about 40 mol % of the LNP.

In certain embodiments, the at least one neutral lipid comprises less than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or about 40 mol % of the LNP.

In certain embodiments, the at least one neutral lipid comprises greater than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or about 40 mol % of the LNP.

In certain embodiments, the at least one neutral lipid comprises about 16 mol % of the LNP. In certain embodiments, the at least one neutral lipid comprises less than about 16 mol % of the LNP. In certain embodiments, the at least one neutral lipid comprises greater than about 16 mol % of the LNP.

In certain embodiments, the at least one neutral lipid comprises about 30 mol % of the LNP. In certain embodiments, the at least one neutral lipid comprises less than about 30 mol % of the LNP. In certain embodiments, the at least one neutral lipid comprises greater than about 30 mol % of the LNP.

In certain embodiments, the at least one neutral lipid comprises dioleoylphosphatidylethanolamine (DOPE). In certain embodiments, the at least one neutral lipid comprises distearoylphosphatidylcholine (DSPC). In certain embodiments, the at least one neutral lipid comprises dioleoylphosphatidylcholine (DOPC).

In certain embodiments, the cholesterol comprises about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or about 75 mol % of the LNP.

In certain embodiments, the cholesterol comprises less than about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or about 75 mol % of the LNP.

In certain embodiments, the cholesterol comprises greater than about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or about 75 mol % of the LNP.

In certain embodiments, the cholesterol comprises about 25 mol % of the LNP. In certain embodiments, the cholesterol comprises less than about 25 mol % of the LNP. In certain embodiments, the cholesterol comprises more than about 25 mol % of the LNP.

In certain embodiments, the cholesterol comprises about 46.5 mol % of the LNP. In certain embodiments, the cholesterol comprises less than about 46.5 mol % of the LNP. In certain embodiments, the cholesterol comprises more than about 46.5 mol % of the LNP.

In certain embodiments, the at least one conjugated lipid comprises about 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10.0, 11.0, 12.0, 13.0, 14.0 or about 15.0 mol % of the LNP.

In certain embodiments, the at least one conjugated lipid comprises less than about 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10.0, 11.0, 12.0, 13.0, 14.0 or about 15.0 mol % of the LNP.

In certain embodiments, the at least one conjugated lipid comprises greater than about 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10.0, 11.0, 12.0, 13.0, 14.0 or about 15.0 mol % of the LNP.

In certain embodiments, the at least one conjugated lipid comprises 1,2-dimyristoyl-rac-gly cero-3-methoxypolyethylene glycol-2000 (DMG-PEG₂₀₀₀).

In certain embodiments, the LNP has a molar ratio of (a):(b):(c):(d) of about 35:16:46.5:2.5. In certain embodiments, the LNP has a molar ratio of (a):(b):(c):(d) of about 40:30:25:2.5.

In certain embodiments, the nucleic acid molecule is a therapeutic agent. In certain embodiments, the nucleic acid molecule comprises RNA. In certain embodiments, the nucleic acid molecule comprises DNA. In certain embodiments, the nucleic acid molecule comprises mRNA. In certain embodiments, the nucleic acid molecule comprises cDNA. In certain embodiments, the nucleic acid molecule comprises miRNA. In certain embodiments, the nucleic acid molecule comprises siRNA. In certain embodiments, the nucleic acid molecule comprises modified RNA.

In certain embodiments, the nucleic acid is mRNA.

In certain embodiments, the LNP has a mass ratio of (a):mRNA of about 20:1, 19:1, 18:1, 17:1, 16:1, 15:1, 14:1, 13:1, 12:1, 11:1, 10:1, 9:1, 8:1, 7:1, 6:1, or about 5:1 (w/w). In certain embodiments, the LNP has a mass ratio of (a):mRNA of about 10:1.

In certain embodiments, mRNA encodes a chimeric antigen receptor (CAR). In certain embodiments, CAR is specific for binding to a surface antigen of a pathogenic cell or a tumor cell. In certain embodiments, the surface antigen is selected from the group consisting of CDI, CD2, CD3. CD5, CD7, CD8, CD16, CD19, CD20, CD22, CD25, CD26, CD27, CD28, CD30, CD33, CD38, CD39, CD40L, CD44, CD45, CD62L, CD69, CD73, CD80, CD83, CD86, CD95, CD103, CD119, CD123, CD126, CD150, CD153, CD154, CD161, CD183, CD223, CD254, CD275, CD45RA, CXCR3, CXCR5, FasL, IL18R1, CTLA-4, OX40, GITR, LAG3, ICOS, PD-1, leu-12, TCR, TLR1, TLR2, TLR3, TLR4, TLR6, NKG2D, CCR, CCR1, CCR2, CCR4, CCR6, CCR7, k light chain, ROR1, ErbB2, ErbB3, ErbB4, EGFR vIII, carcinoembryonic antigen, EGP2, EGP40, mesothelin, TAG72, PSMA, NKG2D ligands, B7-H6, IL13R-α2, MUC1, VEGF-A, Tem8, FAP, EphA2, HER2, MUC16, CA9, GD2, GD3, HMW-MAA, CD171, Lewis Y, G250/CALX, HLA-AI MAGE A1, HAL-A2 NY-ESO-1, PSC1, folate receptor-α, 8H9, NCAM, VEGF, 5T4, Fetal AchR, NKG2D ligands, TEM1, and TEM8.

In certain embodiments, the mRNA encodes an enzyme. In certain embodiments, the mRNA encodes a clustered regularly interspaced short palindrome repeats (CRISPR) associated protein, optionally wherein the CRISPR associated protein is Cas9. In certain embodiments, the mRNA further encodes a small-guiding RNA (sgRNA).

### Methods

In another aspect, the present disclosure provides a method of treating, preventing, and/or ameliorating a disease in a subject. In certain embodiments, the method comprises administering to the subject at least one lipid nanoparticle (LNP) of the present disclosure and/or at least one pharmaceutical composition of the present disclosure.

In certain embodiments, the LNPs of the present disclosure are useful for the treatment, prevention, and/or amelioration of any of a number of diseases and/or disorders. In certain embodiments, the LNPs of the present disclosure are suitable for treatment, prevention, and/or amelioration of diseases and/or disorders for which mRNA delivery and/or gene therapy is useful (e.g., cancer immunotherapy). In certain embodiments, the LNPs of the present disclosure are suitable for the treatment, prevention, and/or amelioration of diseases and/or disorders including autoimmune disorders, cardiovascular diseases, and neurological disorders, inter alia. In certain embodiments the LNPs of the present disclosure are suitable for gene editing applications, including gene editing of the liver, brain, lung, and/or hematopoietic cells for monogenic diseases.

In certain embodiments, the disease is cancer. In certain embodiments, the cancer is at least one selected from the group consisting of pancreatic cancer, colorectal cancer, bladder cancer, breast cancer, prostate cancer, renal cancer, hepatocellular cancer, lung cancer, ovarian cancer, cervical cancer, gastric cancer, esophageal cancer, head and neck cancer, melanoma, neuroendocrine cancer, CNS cancer, brain cancer, bone cancer, soft tissue sarcoma, non-small cell lung cancer, small-cell lung cancer, or colon cancer. In certain embodiments, the subject is further administered at least one additional agent or therapy useful for treating, preventing, and/or ameliorating cancer in the subject.

In certain embodiments, the subject is a mammal. In certain embodiments, mammal is a human.

In another aspect, the present disclosure provides a method of delivering a nucleic acid or therapeutic agent to the liver of a subject. In certain embodiments, the method comprises administering to the subject at least one lipid nanoparticle (LNP) of the present disclosure and/or at least one pharmaceutical composition of the present disclosure.

In another aspect, the present disclosure provides a method of preparing a modified immune cell or precursor thereof. In certain embodiments, the method comprises contacting an immune cell or precursor thereof with at least one lipid nanoparticle (LNP) of the present disclosure and/or at least one pharmaceutical composition of the present disclosure.

In certain embodiments, the modified immune cell or precursor thereof is αβ T cell, a γδ T cell, a CD8+ T cell, a CD4+ helper T cell, a CD4+ regulatory T cell, an NK T cell, an NK cell, and any combination thereof.

In certain embodiments, the modified immune cell or precursor thereof is a T cell, optionally wherein the T cell is a CD4+ T cell. In certain embodiments, the modified immune cell or precursor thereof is a NK cell.

In another aspect, the present disclosure provides a method of preparing a compound of Formula (2-2), or a salt, solvate, stereoisomer, or isotopologue thereof:

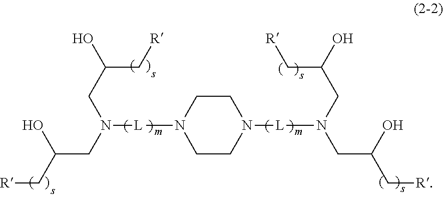

In certain embodiments, the method comprises contacting a compound of formula (1-4):

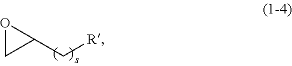

and a compound of formula (2-1):

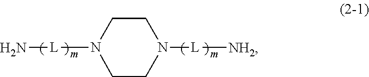

wherein:
each occurrence of L is independently selected from the group consisting of a bond, -(optionally substituted C₁-C₁₂ alkylenyl)-X—, -(optionally substituted C₂-C₁₂ alkenylenyl)-X—, -(optionally substituted C₁-C₁₂ alkynylenyl)-X—, -(optionally substituted C₁-C₁₂ heteroalkylenyl)-X—, optionally substituted C₃-C₈ cycloalkylenyl, and optionally substituted C₂-C₈ heterocyloalkylenyl;
each occurrence of R′ is independently optionally substituted C₁-C₁₂ alkyl, wherein each occurrence of R′ comprises at least one tertiary carbon;
each occurrence of m is independently an integer selected from the group consisting of 1, 2, 3, and 4; and
each occurrence of s is independently an integer selected form the group consisting of 1, 2, 3, 4, 5, 6, 7, 8, 9, 10, 11, and 12.

In certain embodiments, the contacting occurs in the presence of a solvent, optionally wherein the solvent comprises ethanol (EtOH). In certain embodiments, the contacting occurs at a temperature of about 70° C. to about 90° C., optionally wherein the contacting occurs at a temperature of about 80° C. In certain embodiments, the contacting occurs for a period of about 1 h to about 72 h, optionally wherein the contacting occurs for a period of about 48 h.

In certain embodiments, the compound of formula (1-4) is prepared by contacting a compound of formula (1-3):

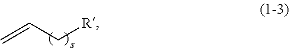

and an organomagnesium compound of formula R′—MgX³,
wherein X³ is selected from the group consisting of Cl, Br, and I.

In certain embodiments, the contacting occurs in the presence of a suitable epoxidation reagent, optionally wherein the epoxidation reagent is m-chloroperoxy benzoic acid. In certain embodiments, the contacting occurs in the presence of a solvent, optionally wherein the solvent comprises dichloromethane.

In certain embodiments, the compound of formula (1-3) is prepared by contacting a compound of formula (1-2):

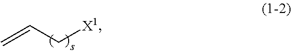

and an organomagnesium compound of formula R′—MgX³, wherein X¹ and X³ are each independently selected from the group consisting of Cl, Br, and I.

In certain embodiments, the contacting occurs in the presence of a suitable catalyst, optionally wherein the catalyst comprises Li₂CuCl₄. In certain embodiments, the contacting occurs in the presence of a solvent, optionally wherein the solvent comprises at least one selected from the group consisting of tetrahydrofuran (THF) and N-methyl pyrrolidone (NMP).

In certain embodiments, the compound of formula (1-2) is prepared by contacting a compound of formula (1-1):

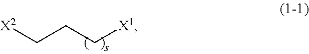

and an alkoxide base, wherein X¹, X², and X³ are each independently selected from the group consisting of Cl, Br, and I.

In certain embodiments, the contacting occurs in the presence of a solvent, optionally wherein the solvent comprises THF. In certain embodiments, the alkoxide base comprises a tert-butoxide base, optionally wherein the tert-butoxide base is potassium tert-butoxide. In certain embodiments, the contacting occurs at a temperature of about 60° C. to about 80° C.

### Pharmaceutical Compositions

In another aspect, the present disclosure provides a pharmaceutical composition comprising the lipid nanoparticle (LNP) of the present disclosure and at least one pharmaceutically acceptable carrier. In certain embodiments, the composition further comprises at least one adjuvant.

Such a pharmaceutical composition may consist of at least one composition of the invention, in a form suitable for administration to a subject, or the pharmaceutical composition may comprise at least one composition, and one or more pharmaceutically acceptable carriers, one or more additional ingredients, or any combinations of these. At least one composition of the invention may be present in the pharmaceutical composition in the form of a physiologically acceptable salt, such as in combination with a physiologically acceptable cation or anion, as is well known in the art.

In certain embodiments, the pharmaceutical compositions useful for practicing the method of the invention may be administered to deliver a dose of between 1 ng/kg/day and 100 mg/kg/day. In other embodiments, the pharmaceutical compositions useful for practicing the invention may be administered to deliver a dose of between 1 ng/kg/day and 1,000 mg/kg/day.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for nasal, inhalational, oral, rectal, vaginal, pleural, peritoneal, parenteral, topical, transdermal, pulmonary, intranasal, buccal, ophthalmic, epidural, intrathecal, intravenous, or another route of administration. A composition useful within the methods of the invention may be directly administered to the brain, the brainstem, or any other part of the central nervous system of a mammal or bird. Other contemplated formulations include projected nanoparticles, microspheres, liposomal preparations, coated particles, polymer conjugates, resealed erythrocytes containing the active ingredient, and immunologically-based formulations.

In certain embodiments, the compositions of the invention are part of a pharmaceutical matrix, which allows for manipulation of insoluble materials and improvement of the bioavailability thereof, development of controlled or sustained release products, and generation of homogeneous compositions. By way of example, a pharmaceutical matrix may be prepared using hot melt extrusion, solid solutions, solid dispersions, size reduction technologies, molecular complexes (e.g., cyclodextrins, and others), microparticulate, and particle and formulation coating processes. Amorphous or crystalline phases may be used in such processes.

The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology and pharmaceutics. In general, such preparatory methods include the step of bringing the active ingredient into association with a carrier or one or more other accessory ingredients, and then, if necessary or desirable, shaping or packaging the product into a desired single-dose or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

In certain embodiments, the compositions of the invention are formulated using one or more pharmaceutically acceptable excipients or carriers. In certain embodiments, the pharmaceutical compositions of the invention comprise a therapeutically effective amount of at least one compound of the invention and a pharmaceutically acceptable carrier. Pharmaceutically acceptable carriers, which are useful, include, but are not limited to, glycerol, water, saline, ethanol, recombinant human albumin (e.g., RECOMBUMIN®), solubilized gelatins (e.g., GELOFUSINE®), and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), recombinant human albumin, solubilized gelatins, suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, isotonic agents, for example, sugars, sodium chloride, or polyalcohols such as mannitol and sorbitol, are included in the composition. Prolonged absorption of the injectable compositions may be brought about by including in the composition an agent that delays absorption, for example, aluminum monostearate or gelatin.

Formulations may be employed in admixtures with conventional excipients, i.e., pharmaceutically acceptable organic or inorganic carrier substances suitable for oral, parenteral, nasal, inhalational, intravenous, subcutaneous, transdermal enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring, and/or fragrance-conferring substances and the like. They may also be combined where desired with other active agents, e.g., other analgesic, anxiolytics or hypnotic agents. As used herein, “additional ingredients” include, but are not limited to, one or more ingredients that may be used as a pharmaceutical carrier.

The composition of the invention may comprise a preservative from about 0.005% to 2.0% by total weight of the composition. The preservative is used to prevent spoilage in the case of exposure to contaminants in the environment. Examples of preservatives useful in accordance with the invention include but are not limited to those selected from the group consisting of benzyl alcohol, sorbic acid, parabens, imidurea and any combinations thereof. One such preservative is a combination of about 0.5% to 2.0% benzyl alcohol and 0.05-0.5% sorbic acid.

The composition may include an antioxidant and a chelating agent that inhibit the degradation of the compound. Antioxidants for some compounds are BHT, BHA, alpha-tocopherol and ascorbic acid in the exemplary range of about 0.01% to 0.3%, or BHT in the range of 0.03% to 0.1% by weight by total weight of the composition. The chelating agent may be present in an amount of from 0.010% to 0.5% by weight by total weight of the composition. Exemplary chelating agents include edetate salts (e.g. disodium edetate) and citric acid in the weight range of about 0.01% to 0.20%, or in the range of 0.02% to 0.10% by weight by total weight of the composition. The chelating agent is useful for chelating metal ions in the composition that may be detrimental to the shelf life of the formulation. While BHT and disodium edetate are exemplary antioxidant and chelating agent, respectively, for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethyl cellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, acacia, and ionic or non-ionic surfactants. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying. Methods for mixing components include physical milling, the use of pellets in solid and suspension formulations and mixing in a transdermal patch, as known to those skilled in the art.

### Administration/Dosing

The regimen of administration may affect what constitutes an effective amount. The therapeutic formulations may be administered to the patient either prior to or after the onset of a disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present disclosure to a patient, such as a mammal, such as a human, may be carried out using known procedures, at dosages and for periods of time effective to treat a disease or disorder contemplated herein. An effective amount of therapeutic (i.e., composition) necessary to achieve a therapeutic effect may vary according to factors such as the activity of the particular therapeutic employed; the time of administration; the rate of excretion of the composition; the duration of the treatment; other drugs, compounds or materials used in combination with the composition; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic composition of the disclosure is from about 0.01 mg/kg to 100 mg/kg of body weight/per day of active agent (i.e., nucleic acid). One of ordinary skill in the art would be able to study the relevant factors and make the determination regarding the effective amount of the therapeutic composition without undue experimentation.

The composition may be administered to an animal as frequently as several times daily, or it may be administered less frequently, such as once a day, once a week, once every two weeks, once a month, or even less frequently, such as once every several months or even once a year or less. It is understood that the amount of composition dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon a number of factors, such as, but not limited to, type and severity of the disease being treated, and type and age of the animal.

Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic composition to produce the desired therapeutic effect in association with the required pharmaceutical vehicle. The dosage unit forms of the disclosure are dictated by and directly dependent on (a) the unique characteristics of the therapeutic composition and the particular therapeutic effect to be achieved, and (b) the limitations inherent in the art of compounding/formulating such a therapeutic composition for the treatment of a disease or disorder in a patient.

In certain embodiments, the compositions of the disclosure are administered to the patient in dosages that range from one to five times per day or more. In other embodiments, the compositions of the disclosure are administered to the patient in range of dosages that include, but are not limited to, once every day, every two days, every three days to once a week, and once every two weeks. It will be readily apparent to one skilled in the art that the frequency of administration of the various combination compositions of the disclosure will vary from subject to subject depending on many factors including, but not limited to, age, disease or disorder to be treated, gender, overall health, and other factors. Thus, the disclosure should not be construed to be limited to any particular dosage regime and the precise dosage and composition to be administered to any patient will be determined by the attending physician taking all other factors about the patient into account.

The amount of active agent of the composition(s) of the disclosure for administration may be in the range of from about 1 μg to about 7,500 mg, about 20 μg to about 7,000 mg, about 40 μg to about 6,500 mg, about 80 μg to about 6,000 mg, about 100 μg to about 5,500 mg, about 200 μg to about 5,000 mg, about 400 μg to about 4,000 mg, about 800 μg to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments there-in-between.

In some embodiments, the dose of active agent (i.e., nucleic acid) present in the composition of the disclosure is from about 0.5 μg and about 5,000 mg. In some embodiments, a dose of active agent present in the composition of the disclosure used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of the composition of the disclosure, alone or in combination with a second pharmaceutical agent; and instructions for using the compound to treat, prevent, or reduce one or more symptoms of a disease or disorder in a patient.

The term “container” includes any receptacle for holding the pharmaceutical composition or for managing stability or water uptake. For example, in certain embodiments, the container is the packaging that contains the pharmaceutical composition, such as liquid (solution and suspension), semisolid, lyophilized solid, solution and powder or lyophilized formulation present in dual chambers. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition.

Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a disease or disorder in a patient.

### Administration

Routes of administration of any of the compositions of the disclosure include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, epidural, intrapleural, intraperitoneal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, emulsions, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Injectable formulations may also be prepared, packaged, or sold in devices such as patient-controlled analgesia (PCA) devices.

Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In certain embodiments of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form in a recombinant human albumin, a fluidized gelatin, in a liposomal preparation, or as a component of a biodegradable polymer system.

Compositions for sustained release or implantation may comprise pharmaceutically acceptable polymeric or hydrophobic materials such as an emulsion, an ion exchange resin, a sparingly soluble polymer, or a sparingly soluble salt.

### EXAMPLES

Various embodiments of the present application can be better understood by reference to the following Examples which are offered by way of illustration. The scope of the present application is not limited to the Examples given herein.

### Materials and Methods

### Materials

All non-IL LNP lipid excipients as well as lipids to make the artificial endosomes were purchased from Avanti Polar Lipids (Alabaster, AL, USA). Cas9 and firefly luciferase mRNA were purchased from TriLink Biotechnologies (San Diego, CA, USA) with 5-methoxyuridine substitutions. TTR sgRNA was synthesized by Axolabs (Kulmbach, Germany), using the following sequence: 5′-ususasCAGCCACGUCUACAGCAGUUUUAGAgcuagaaauagcAAGUUAAAAUAAGGCU AGUCCGUUAUCAacuugaaaaaguggcaccgagucggugcusususu-3′ (SEQ ID NO: 1), where N refers to RNA residues, n are 2′-O-methyl residues, and s are phosphorothioate backbone modifications.

The chemicals 1,2-epoxyoctane, 1,2-epoxydecane, 1,2-epoxydodecane, 1,2-epoxytetradecane, and 8-bromo-1-octene were purchased from TCI (Montgomeryville, PA, USA); Triton X-100 was purchased from Alfa Aesar (Haverhill, MA, USA), 1,12-dibromododecane and N1-(2-(4-(2-aminoethyl)piperazin-1-yl)ethyl)ethane-1,2-diamine were purchased from AmBeed (Arlington Heights, IL, USA); anhydrous 1-methyl-2-pyrrolidinone and chloroform-d were purchased from Acros Organics (Geel, Belgium); all non-anhydrous solvents as well as anhydrous magnesium sulfate, and 1 N hydrochloric acid were purchased from Fisher Scientific (Waltham, MA, USA); 10-bromo-1-decene was purchased from Oakwood Chemicals (Estill, SC, USA); 6-bromo-1-hexene was purchased from Asta Tech (Bristol, PA, USA); 2-{2-[4-(2-{[2-(2-aminoethoxy)ethyl]amino}ethyl)piperazin-1-yl]ethoxy}ethan-1-amine was purchased from Enamine (Kiev, Ukraine). All other chemical reagents were purchased from MilliporeSigma (St. Louis, MO, USA).

### Synthesis

All flash chromatography was performed on a Teledyne Isco (Lincoln, NE, USA) CombiFlash NextGen 300+ equipped with evaporative light scattering detection using RediSep Gold® silica gel disposable flash columns. Solvent evaporation was performed using a Buchi (New Castle, DE, USA) Rotavapor® R-300 System Professional. ¹H and ¹³C NMR spectra were acquired in d-chloroform using an Avance Neo 400 MHz spectrometer (Bruker, Billerica, MA, USA). LC-MS spectra were acquired in ethanol using an SQD equipped with an Acquity UPLC (Milford, MA, USA), using a C8 column with a 2 min wash followed by a gradient mobile phase from 50% water (1% trifluoroacetic acid) and 50% acetonitrile (1% trifluoroacetic acid) to 100% acetonitrile (1% trifluoroacetic acid).

12-bromnododec-1-ene

To a 250 mL round bottom flask was added 1,12-dibromododecane (8.00 g, 24.4 mmol, 2.0 equiv.) and anhydrous tetrahydrofuran (20 mL). Then, potassium tert-butoxide (2.74 g, 24.4 mmol, 1.0 equiv.) in anhydrous THF (50 mL) was added dropwise. The reaction stirred at 70° C. for 16 h. The reaction was then quenched with deionized water (40 mL), and the was extracted with hexanes (3×35 mL). The organic fractions were combined, dried with magnesium sulfate, filtered, and concentrated in vacuo. The crude product was further purified via flash chromatography assisted by a CombiFlash using a liquid injection into an 80 g column. The mobile phase was isocratic hexanes for 10 min using a 20 mL/min flow. The product was isolated as a clear oil at a 25.8% yield. ¹H NMR (400 MHz, CDCl₃) δ 5.82 (ddt, J=16.9, 10.1, 6.7 Hz, 1H), 5.18-4.81 (m, 2H), 3.41 (td, J=6.8, 1.0 Hz, 2H), 2.25-1.96 (m, 2H), 1.87 (dt, J=14.5, 7.0 Hz, 2H), 1.64-1.01 (m, 14H). ¹³C NMR (101 MHz, CDCl₃) δ 139.06, 114.12, 33.83, 33.77, 32.89, 29.52, 29.48, 29.46, 29.15, 28.96, 28.80, 28.21.

### General Procedure “A”: Branched Alkene Synthesis

A 100 mL Schlenk flask was purged with nitrogen. To the flask was added anhydrous tetrahydrofuran (5 or 10 mL), N-methylpyrrolidinone (48.0 mmol, 4.0 equiv.), dilithium tetrachlorocuprate (0.1 M tetrahydrofuran; 0.36 mmol, 0.03 equiv.), and the corresponding bromoalkene (12.0 mmol, 1.0 equiv.). The solution stirred at room temperature and under nitrogen for 5 min. Then, the flask was put on a water bath at room temperature. The corresponding Grignard reagent (13.2 mmol, 1.1 equiv.) was then added dropwise. After 5 min, the flask was removed from the water bath, and then stirred at room temperature for 1 h. The flask was then cooled to 0° C. and then slowly quenched with hydrochloric acid (1 M; 40 mL). The aqueous phase was extracted with hexanes (3×20 mL), and the organic layers were combined, washed with hydrochloric acid (1 M; 1×40 mL), washed with brine (2×40 mL), dried with magnesium sulfate, filtered, and concentrated in vacuo. The crude product was further purified via flash chromatography assisted by a CombiFlash using a liquid injection into a 40 g column. The mobile phase was isocratic hexanes using a flow rate of 7 mL/min. The products were isolated as clear oils. See the Supplementary Information for the specific conditions for each reaction.

### General Procedure “B”: Branched Epoxide Synthesis

To a 100 mL round bottom flask was added the corresponding branched alkene (1.0 equiv.) and dichloromethane (5 mL). The flask was mixed for 1 min and then cooled to 0° C. Then, to the flask was added dropwise half of a solution of meta-chloroperoxybenzoic acid (70% pure; 2.0 equiv.) dissolved in dichloromethane (30 mL). The mixture stirred for 1 h after which the other half of the meta-chloroperoxybenzoic acid solution in dichloromethane was added dropwise. After 1 h, the reaction flask was removed from the 0° C. bath and stirred at room temperature for 14 h. The reaction was quenched by adding 20 mL of a 1:1 solution of sat. sodium bicarbonate and sat. sodium thiosulfate. The layers were separated, and the organic layer was washed with brine (1×30 mL). The aqueous layers were then combined and extracted with DCM (3×15 mL). The organic layers were combined, dried with magnesium sulfate, filtered, and concentrated in vacuo. The crude product was further purified via flash chromatography assisted by a CombiFlash using a liquid injection into a 24 g column. The mobile phase had a gradient of 100% hexanes to 90% hexanes and 10% ethyl acetate over 15 min using a flow rate of 35 mL/min. The products were isolated as clear oils. See the Supplementary Information for the specific conditions for each reaction.

### General Procedure “C”: Ionizable Lipid Synthesis

To a 1 dram vial was added the corresponding polyamine core (1.0 equiv.), the corresponding epoxide (6.0 or 7.0 equiv.), and ethanol (0.3 mL). The reaction stirred at 80° C. for 48 h. Afterwards, the solution was diluted with dichloromethane (0.7 mL). The solution was purified via flash chromatography assisted by a CombiFlash using a liquid injection into a 12 g column. The mobile phase had a gradient of 95% dichloromethane and 5% Ultra solution (75% dichloromethane, 22% methanol, and 3% aqueous ammonium hydroxide) to 80% dichloromethane and 20% Ultra solution over 35 min using a flow rate of 7 mL/min. The products were isolated as yellow to clear viscous oils. See the Supplementary Information for the specific conditions for each reaction.

### LNP Formulation

An ethanol phase containing each lipid used in the formulation (i.e., ionizable lipid, neutral lipid, and lipid conjugate) and cholesterol, and an aqueous phase containing mRNA were mixed using a microfluidic device to formulate the LNPs. The ethanol phase consisted of the corresponding ionizable lipid, 1,2-dioleoyl-sn-glycero-3-phosphoethanolamine (DOPE), cholesterol and 1,2-dimyristoyl-sn-glycero-3-phosphoethanolamine-N-[methoxy(polyethyleneglycol)-2000](DMG-PEG2000) with a fixed molar ratio of 35%, 16%, 46.5%, and 2.5%, respectively. The aqueous phase was composed of mRNA dissolved in 10 mM citrate buffer. The aqueous and ethanol phases were mixed at flow rates of 1.8 mL/min and 0.6 mL/min, respectively, using Pump33DS syringe pumps. LNPs were dialyzed in 1× phosphate buffered saline using a microdialysis cassette (20,000 MWCO, Thermo Fisher Scientific, Waltham, MA) for 2 h and then filtered through a 0.22 μm filter.

### LNP Encapsulation Efficiency

mRNA encapsulation efficiencies of each LNP formulation were calculated using the Quant-iT-RiboGreen (Thermo Fisher Scientific, Waltham, MA) assay as previously described (Heyes et al., 2005, J. Controlled Release. 107:276-287). Each LNP sample was diluted to approximately 2 ng/μL in two microcentrifuge tubes containing 1×TE buffer or 0.1% (v/v) Triton X-100 (Sigma-Aldrich). After 20 min, LNPs in TE buffer and Triton X-100 as well as mRNA standards were plated in triplicate in black 96-well plates and the fluorescent RiboGreen reagent was added per manufacturer's instructions. Fluorescence intensity was read on an Infinite 200 Pro plate reader (Tecan) at an excitation wavelength of 480 nm and an emission wavelength of 520 nm. RNA content was estimated by comparison to a standard curve estimated using least squares linear regression (LSLR). Encapsulation efficiency was calculated as (B−A)/B·100 where A is the RNA content in TE buffer and B is the RNA content in Triton X-100. Encapsulation efficiencies are reported as mean±standard deviation (n=3).

### Dynamic Light Scattering and Zeta Potential

For baseline dynamic light scattering (DLS) measurements, 10 μL of each LNP solution was diluted 100× in 1×PBS in 4 mL disposable cuvettes. For baseline zeta potential measurements, 20 μL of each LNP solution was diluted 50× in deionized water in DTS1070 zeta potential cuvettes (Malvern Panalytical, Malvern, UK). Four measurements each with at least 10 runs were recorded for each sample using a Zetasizer Nano (Malvern Instruments, Malvern, UK). Data are reported as mean±standard deviation (n=3 to 4 measurements).

### LNP pKa Measurements

Surface ionization measurements to calculate the pKa of each LNP formulation were performed as previously described (Hajj et al., 2019, Small, 15:1805097). Buffered solution containing 150 mM sodium chloride, 20 mM sodium phosphate, 20 mM ammonium acetate, and 25 mM ammonium citrate was adjusted to pH 2 to 12 in increments of 0.5. 125 μL of each pH-adjusted solution and 5 μL of each LNP formulation were plated in triplicate in black 96-well plates. 6-(p-toluidinyl)naphthalene-2-sulfonic acid (TNS) was then added to each well to a final TNS concentration of 6 μM. The fluorescence intensity was read on an Infinite 200 Pro plate reader (Tecan, Morrisville, NC) at an excitation wavelength of 322 nm and an emission wavelength of 431 nm. Using least squares regression, the pKa was taken as the pH corresponding to half-maximum fluorescence intensity, i.e., 50% protonation.

In Vitro LNP-Mediated Luciferase mRNA Delivery to HeLa Cells HeLa cells (ATCC no. CCL-2) were cultured in DMEM with L-glutamine (Thermo Fisher Scientific) supplemented with 10% FBS and T % penicillin-streptomycin. Cells were plated at 20,000 cells per well in 100 μL of medium in tissue culture treated 96-well plates and were left to adhere overnight. LNP formulations were used to treat cells at a dose of 20 ng of mRNA (i.e., TriLink Luciferase) per 20,000 cells. One group of cells was designated as untreated and was treated with media only. 24 hours after treatment with LNPs, the media was removed. 50 μL of 1× lysis buffer (Promega, Madison, WI) followed by 100 μL of luciferase assay substrate (Promega) was added to each well. After 10 minutes of incubation, luminescence was quantified using an Infinite 200 Pro plate reader (Tecan). The luminescence signal for each condition was normalized by dividing by the luminescence signal from an LNP designated as a control (i.e., C14-494).

To evaluate cytotoxicity, additional plates were prepared as described elsewhere herein. After 24 hours, 100 μL of CellTiter-Glo (Promega) was added to each well and the luminescence corresponding to ATP production was quantified using a plate reader following 10 minutes of incubation. Luminescence for each group was normalized by dividing by the luminescence signal from untreated control cells.

Luciferase expression and percent viability are reported as mean standard deviation (n=3-4 biological replicates and at least 4 technical replicates per plate). For luciferase expression, a 2-way ANOVA with a Dunnett's multiple comparisons test was used to compare means across formulation and treatment condition.

### Luciferase Imaging and Quantification

Luciferase signal was assessed in Black 6 mice following intravenous tail vein injection containing luciferase mRNA. Specifically, mice were imaged 12 h after I.V. injection of LNPs or PBS. Luciferase imaging was performed using an in vivo imaging system (IVIS, PerkinElmer, Waltham, MA). Ten minutes before sacrifice and imaging, the mice were injected intraperitoneally with D-luciferin at 150 mg/kg and potassium salt (Biotium, Fremont, CA). The mouse liver, spleen, lungs, kidneys, and heart were subsequently removed and imaged by IVIS. Image analysis was conducted using the Living Image software (PerkinElmer). Reported total body and organ bioluminescence represent the mean±standard deviation (SD) (n≥3). The representative organ IVIS images shown are those that have the highest luminescence values for each treatment condition.

### In Vivo Biodistribution Studies

C57BL/6J female mice of 6-8 weeks old with an average weight of 20 g were purchased from Jackson Laboratory (Bar Harbor, ME). Mice were injected with LNPs encapsulating fluc mRNA via the lateral tail vein at a dose of 0.1 mg of mRNA per kg of body mass (mg/kg). After 12 h, the hair on the ventral side of the mice was removed with Veet Gel Cream Hair Remover (Reckitt Benckiser, Slough, UK). The mice were then administered an intraperitoneal injection of D-luciferin (0.2 mL, 15 mg/mL; Biotium, Fremont, CA). After 5 min, full body luminescence images were obtained using an In Vivo Imaging System (IVIS; PerkinElmer, Waltham, MA). Afterwards. The mice were euthanized, and the heart, lungs, liver, kidneys, and spleen were removed and imaged for luminescence using IVIS. Luminescence flux was quantified by the Living Image Software (PerkinElmer) by placing rectangular region of interests (ROI) around the full body or organ images, keeping the same ROI sizes among each body or organ. Total flux was reported as mean±SEM of n=3 biological replicates.

### TTR Gene Editing

One day prior to injections, blood was collected from mice via retro-orbital bleeding. Serum was isolated by centrifuging the blood in Microtainer blood collection tubes containing serum separator gel (BD, Franklin Lakes, NJ, USA) for 15 min at 3,500 rpm. Mice were injected with LNPs encapsulating Cas9 mRNA and TTR sgRNA at a 1:3 molar ratio via the lateral tail vein at a dose of 1.0 mg/kg. After 7 days, serum was isolated as described previously, the mice were euthanized, and the livers were removed. Serum TTR levels were measured using a mouse Prealbumin ELISA Kit (Aviva Systems Biology, San Diego, CA, USA) according to the manufacturer's instructions.

For indel analysis, DNA was extracted from the liver using a DNeasy Blood & Tissue Kit (Qiagen, Hilden, Germany) and quantified using a nanodrop plate attachment on an Infinite 200 Pro plate reader (Tecan). PCR amplification of the TTR target site was carried out using Q5 High-Fidelity DNA Polymerase (New England Biolabs. Ipswich, MA, USA) and the following primer sequences: mTTR-exon2-F, 5′-CGGTTTACTCTGACCCATTTC-3′ (SEQ ID NO:2) and mTTR-exon2-R, 5′-GGGCTTTCTACAAGCTTACC-3′ (SEQ ID NO:3). Deep sequencing of the TTR amplicons and determination of the on-target indel frequency was performed essentially as described except that 150 bp pair end reads were produced.

### Primary T Cell Luminescence

In certain embodiments the present disclosure provides primary T cell luminescence data obtained using health human donor T cells to evaluate the LNPs of the present disclosure. Cells were plated at 60,000 cells per well with 200 ng of mRNA (i.e., TriLink Luciferase) per well and treated with the LNPs of the present disclosure.

One group of cells was designated as untreated and was treated with medium only. 24 hours after treatment with LNPs, the cells were centrifuged for 5 min at 700 g and the media was removed. 50 μL of 1× lysis buffer (Promega, Madison, WI) followed by 100 μL of luciferase assay substrate (Promega) was added to each well. After 10 minutes of incubation, luminescence was quantified using an Infinite 200 Pro plate reader (Tecan). The luminescence signal for each condition was normalized by dividing by the luminescence signal from an LNP designated as a control (i.e., C14-494). For luciferase expression, a 1-way ANOVA multiple comparisons test with Sidak multiple comparison correction was performed using the B10 formulation was the control group. Additionally, a 2-way ANOVA with a Dunnett's multiple comparisons test was used to compare means across formulation and treatment condition.

To evaluate cytotoxicity, additional plates were prepared as described elsewhere herein. After 24 hours, the cells were centrifuged for 5 min at 700 g, and 100 μL of CellTiter-Glo (Promega) was added to each well and the luminescence corresponding to ATP production was quantified using a plate reader following 10 minutes of incubation. Luminescence for each group was normalized by dividing by the luminescence signal from untreated control cells.

### Cryogenic Transmission Electron Microscopy

Morphology and size was analyzed by cryo-TEM by adding 3 μL of the LNPs at an mRNA concentration of 50 ng/μL were applied to a Quantifoil holey carbon grid which had been glow discharged. Grids were blotted and plunge frozen in liquid ethane using a Vitrobot Mark IV. Imaging was performed at the Beckman Center for cryo-EM on a Titan Krios equipped with a K3 Bioquantum.

### LNP Stability Analysis

LNPs were diluted 10-fold in either 1×PBS or supplemented DMEM (Gibco). The hydrodynamic diameter and PDI of the LNPs were measured every hour at 37° C. using a DynaPro Plate Reader III (Wyatt Technology), as described above. All samples were run in duplicate. Supplemented DMEM was measured as a control.

### Kupffer Cell Knockdown

Mouse macrophage cells were depleted by administering 0.2 mL of clodronate liposomes (Liposoma, Amsterdam, Netherlands) at a dose of 5 mg/mL via the lateral tail vein. After 24 h, the mice were reinjected via the lateral tail vein with LNPs encapsulated fluc at a dose of 0.1 mg/kg. After 12 h, liver luminescence was quantified as described above.

To verify that Kupffer cells were depleted, a subset of mice were injected with clodronate liposomes as described above. After 24 h, the livers were perfused with 10 mL of 1×PBS followed by 10 mL of supplemented DMEM containing 5 mg/mL collagenase IV (STEMCELL Technologies, Vancouver, Canada). The livers were then dissected and placed in 5 mL of RPMI with 5 mg/mL of collagenase IV for 1 h at room temperature. The livers lobes were then separated, crushed using a syringe slider, and passed through a 70 μm filter. The suspension was then centrifuged for 3 min at 100 rpm to pellet the hepatocytes. The supernatant was transferred to a new tube and up to 30 mL of supplemented DMEM was added. The centrifugation process was repeated twice more. Afterwards, the three separate supernatants were spun down at 200 rpm for 10 min, and afterwards, the top portion of the supernatant was discarded, leaving ~5 mL left in each tube. The fractions were combined into a single tube, spun at 25 g for 5 min, the cell pellet was washed with 6 mL of supplemented DMEM, recentrifuged, and the top half of the supernatant was collected. The cells were counted as described above, and then analyzed via flow cytometry.

### LNP Accumulation

LNPs were reformulated with fluc mRNA as described above. Afterwards, the LNPs were mixed with 1,1′-dioctadecyl-3,3,3′,3′-tetramethylindotricarbocyanine iodide (DiR; 5 μM in DMSO; Thermo Fisher Scientific) at a volume ratio of 50:1, respectively. The solution was placed on a plate shaker at 200 rpm for 25 min at room temperature. Then, the LNPs were administered into mice and dissected as described above. Organ fluorescence was obtained using the specific “DiR” setting on Living Image. ROIs were obtained as described above. Total radiant efficiency was reported as mean±SEM of n=3 biological replicates.

### Artificial Endosome Disruption Assay

Artificial endosomes were generated via a lipid film hydration method. To a 3 mL vial was added DOPS, DOPC, DOPE, NBD-PE, and Rho-PE at a molar ratio of 25:25:48:1:1. The lipids were concentrated in vacuo with aluminum foil wrapped around the rotary evaporator to prevent photobleaching. After 2 h of concentrating, the samples were rehydrated with 1×PBS (pH 7.4) at a final concentration of 1 mM using a Branson 3800 Ultrasonic Cleaner (Brookfield, CT, USA) at room temperature for 20 min.

The assays were performed in black bottom 96-well plates. To each well were added 0.1 mL PBS (pH 5.5, 0.1 M), 1 μL of the artificial endosome, and an amount of LNP corresponding to 400 ng.

As a negative control, LNP was substituted with more PBS, and as a positive control, the LNP was replaced with 2% Triton-X100. The plate was wrapped in aluminum foil and incubated at 37° C. Fluorescence was measured at various timepoints over the course of 24 h.

### Example 1: Synthesis of Certain Exemplary Ionizable Lipids, Preparation of Lipid Nanoparticle (LNP) Formulations, and Selected Properties Thereof

Ionizable lipids (ILs) can be generated in one step by reacting a monoamine or polyamine with lipids containing electrophilic functional groups (e.g., epoxides). This allows for the rapid production of combinatorial libraries of ILs by simply mixing different amine cores with epoxides of various lengths. Therefore, a synthetic approach was developed to synthesize epoxides with any desired length and terminal branching that could then be further reacted with most polyamine or monoamine reagents to produce ILs with greater structural variability.

To achieve the flexible synthesis of structurally diverse epoxides, primary bromoalkenes were coupled with branched halomagnesiumalkyls via copper-catalyzed Grignard C—C coupling to produce branched alkenes (FIG. 1). Primary bromoalkenes are common reagents and thus are used here to establish the different lengths of the epoxides. Longer bromoalkenes can be generated by monoselective elimination of dibromoalkanes with tert-butoxide. Numerous Grignard functionalized alkyls are also commercially available due to their wide-use in other synthetic avenues and are utilized here to establish the terminal branched group. The terminally branched alkenes were converted into the corresponding branched epoxides via mCBPA-mediated epoxidation. The C—C coupling and epoxidation steps can be completed and purified in less than 24 h, demonstrating the ease of this method.

In this study, twelve unique branched epoxides were synthesized, non-limiting examples including isopropyl, tert-butyl, and sec-butyl branching groups at four different lipid lengths. To form the ILs, the epoxides were initially reacted via an SN2 reaction with 2-(2-aminoethoxy)-N-(2-(4-(2-(2-aminoethoxy)ethyl)piperazin-1-yl)ethyl)ethan-1-amine (494) or N-(2-(4-(2-aminoethyl)piperazin-1-yl)ethyl)ethane-1,2-diamine (200) (FIGS. 2A-2B). The 494 and 200 polyamine cores have been previously used to generate ILs and the corresponding LNPs for several applications, including CAR T-cell therapy, in utero delivery, and delivery to the placenta, and thus this core represents an interesting structural motif to explore these preliminary studies. As controls, four linear ILs of the same relative lipid length as the branched library were also synthesized using the same SN2 reaction. In total, using 494, sixteen exemplary ILs were synthesized.

The nomenclature used herein to described the branched lipids of the present disclosure utilizes the formula “AXb-C”, where A is the reactive moiety of the lipid tail, b is the branching type, C is the lipid core number, and X is the linker length or number of methylene units between A and B. The branching abbreviations are “i” for isopropyl, “t” for tert-butyl, and “s” for sec-butyl. Non-branched ILs utilize their historical name.

In one aspect, the present disclosure relates to lipid nanoparticles comprising one or more ionizable lipid compounds of Formula (I), one or more neutral lipids, cholesterol, and one or more conjugated lipids. In certain embodiments, the one or more conjugated lipids inhibit aggregation of two or more lipid nanoparticles. In certain embodiments, the lipid nanoparticles further comprise one or more nucleic acid cargos. The preparation and properties of such lipid nanoparticles is provided herein in greater detail.

In certain embodiments, LNPs of the present disclosure were prepared using microfluidic devices, to prepare LNPs having the following molar ratio of components: (a) ionizable lipid of Formula (I) (35 mol %); (b) DOPE (16 mol %); (c) cholesterol (46.5 mol %), and DMG-PEG₂₀₀₀ (2.5 mol %). In certain embodiments, the ionizable lipid of Formula (I) is selected from the group consisting of E4i-494, E6i-494, E8i-494, E10i-494, E4t-494, E6t-494, E8t-494, E10t-494, E4s-494, E6s-494, E8s-494, E10s-494, E4i-200, E8i-200, E4t-200, E8t-200, E4s-200, and E8s-200 (Table 1). LNPs comprising each of the aforementioned ionizable lipids of Formula (I) and having the aforementioned molar ratio have been exemplified herein, and such LNP formulations are disclosed herein with reference to the identifier assigned to each ionizable lipid used to prepare the LNP (e.g., E4i-494 ionizable lipid was used to prepare the LNP labeled E4i-494).

Since the present exemplary LNPs used in several of the exemplary applications and/or studies described herein are formulating using the same, non-limiting excipient molar ratio as well as using the same phospholipid and PEGylated lipid, the difference between each LNP formulation is the structure of the IL.

For consistency, each LNP is named after the corresponding IL, as described herein. The LNPs were initially encapsulated with firefly luciferase (fluc) mRNA at a 10:1 weight ratio of IL and mRNA, and were characterized for encapsulation efficiency, hydrodynamic diameter, ζ-potential, and pKₐ (Table 2). Although multiple parameters were evaluated, there were no noticeable differences between the LNPs with linear, isopropyl, tert-butyl, and sec-butyl ILs. All exemplary LNPs have >80% encapsulation efficiency and sizes ranging from 70-160 nm with average PDIs of approximately 0.2. The ζ-potentials of the LNPs also ranged from 5.57 to −28.1, indicating that most have neutral or weakly negative charge. Lastly, the majority of the LNPs have a pKₐ around 6.

TABLE 2
Selected properties of exemplary LNPs of the present disclosure and controls

| LNP | EE (%) | Size (nm) | PDI | ζ-potential | pKa |
|---|---|---|---|---|---|
| C8-494 | 87.6 ± 9.9 | 104 ± 32 | 0.091 ± 0.019 | −6.73 | 7.29 |
| E4i-494 | 91.3 ± 2.8 | 76.0 ± 32.0 | 0.177 ± 0.019 | −3.73 | 6.58 |
| E4t-494 | 89.5 ± 3.9 | 67.0 ± 25.1 | 0.140 ± 0.023 | −10.3 | 6.40 |
| E4s-494 | 89.0 ± 2.5 | 72.7 ± 24.7 | 0.116 ± 0.007 | −8.40 | 6.16 |
| C10-494 | 89.0 ± 4.7 | 89.6 ± 44.4 | 0.256 ± 0.029 | −0.240 | 6.30 |
| E6i-494 | 83.6 ± 11.8 | 81.9 ± 41.5 | 0.256 ± 0.02 | −13.7 | 5.90 |
| E6t-494 | 91.4 ± 2.7 | 122 ± 57 | 0.219 ± 0.031 | −16.5 | 5.79 |
| E6s-494 | 91.5 ± 2.9 | 82.3 ± 44.7 | 0.269 ± 0.032 | −22.1 | 5.84 |
| C12-494 | 89.4 ± 4.3 | 106 ± 66 | 0.388 ± 0.107 | −11.5 | 6.16 |
| E8i-494 | 89.5 ± 3.9 | 108 ± 50 | 0.213 ± 0.036 | −13.1 | 6.22 |
| E8t-494 | 89.0 ± 2.5 | 92.6 ± 39.5 | 0.182 ± 0.021 | −28.1 | 5.83 |
| E8s-494 | 89.0 ± 4.7 | 88.5 ± 44.5 | 0.253 ± 0.034 | 5.47 | 6.19 |
| C14-494 | 83.6 ± 11.8 | 92.6 ± 47.4 | 0.261 ± 0.065 | −2.21 | 6.58 |
| E10i-494 | 91.4 ± 2.7 | 100 ± 44 | 0.196 ± 0.066 | −11.3 | 6.14 |
| E10t-494 | 91.5 ± 2.9 | 155 ± 75 | 0.232 ± 0.028 | −14.4 | 6.34 |
| E10s-494 | 89.4 ± 4.3 | 118 ± 60 | 0.258 ± 0.031 | −18.4 | 6.30 |
| C8-200 | 87.9 ± 5.3 | 104 ± 32 | 0.091 ± 0.019 | −6.73 | 7.61 |
| E4i-200 | 79.2 ± 8.2 | 76.0 ± 32.0 | 0.177 ± 0.019 | −3.73 | 6.91 |
| E4t-200 | 90.4 ± 4.7 | 67.0 ± 25.1 | 0.140 ± 0.023 | −10.3 | 5.15 |
| E4s-200 | 93.8 ± 3.7 | 72.7 ± 24.7 | 0.116 ± 0.007 | −8.40 | 5.50 |
| C12-200 | 85.4 ± 5.1 | 96.7 ± 43.3 | 0.201 ± 0.015 | −18.6 | 5.75 |
| E8i-200 | 87.9 ± 4.3 | 100 ± 37 | 0.136 ± 0.020 | −17.7 | 4.59 |
| E8t-200 | 85.1 ± 6.1 | 98.1 ± 38.9 | 0.157 ± 0.020 | −11.7 | 5.96 |
| E8s-200 | 87.8 ± 4.8 | 99.4 ± 36.1 | 0.132 ± 0.016 | −19.5 | 5.96 |

LNPs C8-494, C10-494, C12-494, and C12-200 correspond to LNPs having the identical formulation to those of the present disclosure, but which differ with respect to the ionizable lipid component, wherein the ionizable lipid was prepared using either polyamine core 494 or polyamine core 200 with an epoxide having a linear alkyl group with a linker length indicated by the C integer (i.e., C10-494 corresponds to an ionizable lipid prepared using polyamine core 494 with 2-undecyloxirane).

### Example 2: LNPs Comprising Branched Ionizable Lipids Enhance In Vivo, Ex Vivo, and In Vitro mRNA Delivery

The present disclosure provides data relating to the in vivo, ex vivo, and in vitro delivery of exemplary LNPs of the present disclosure.

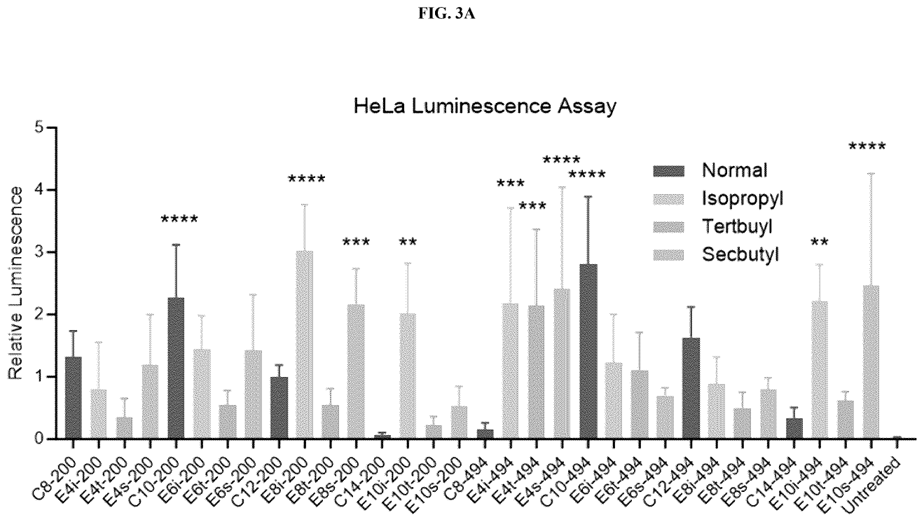

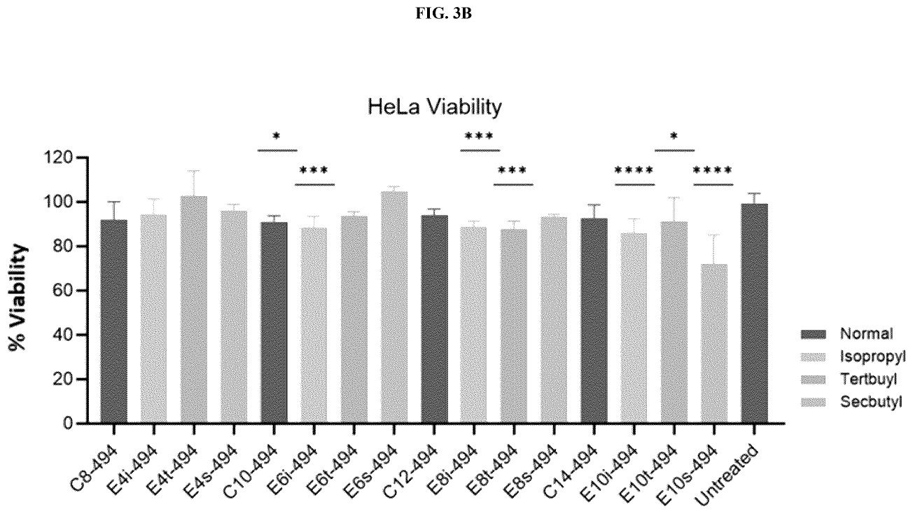

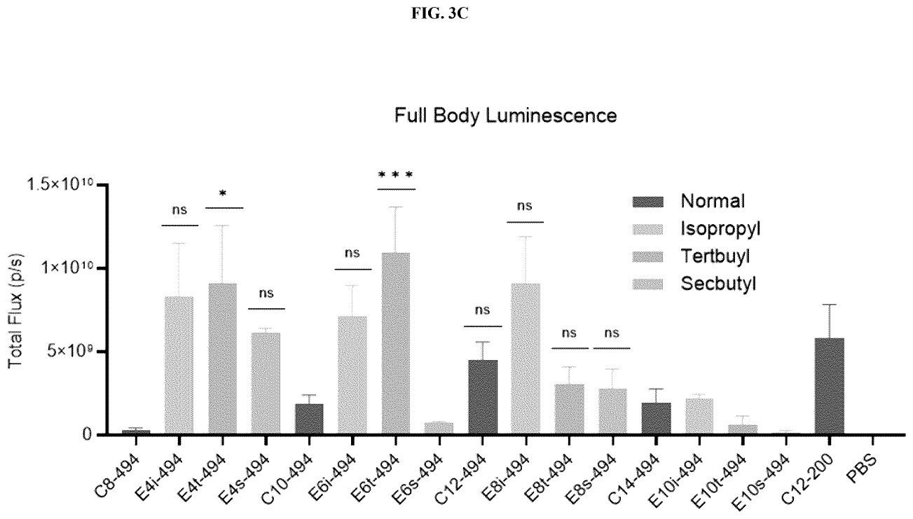

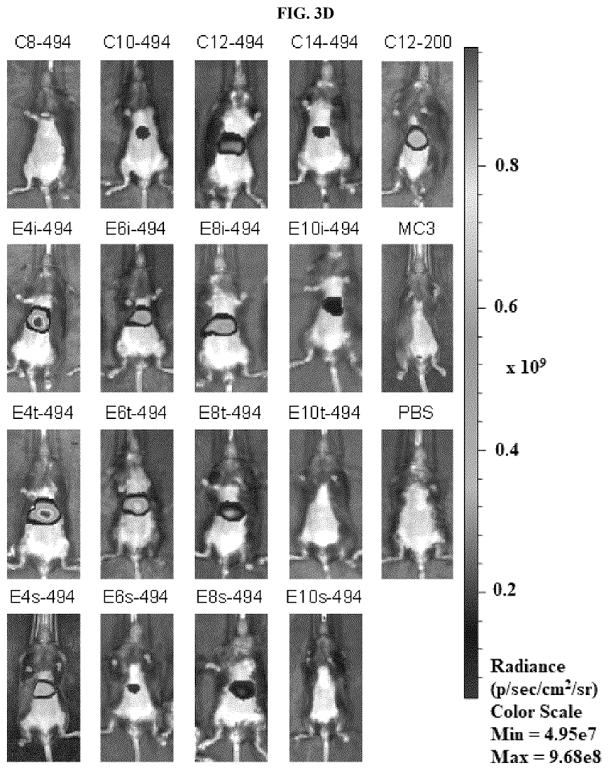

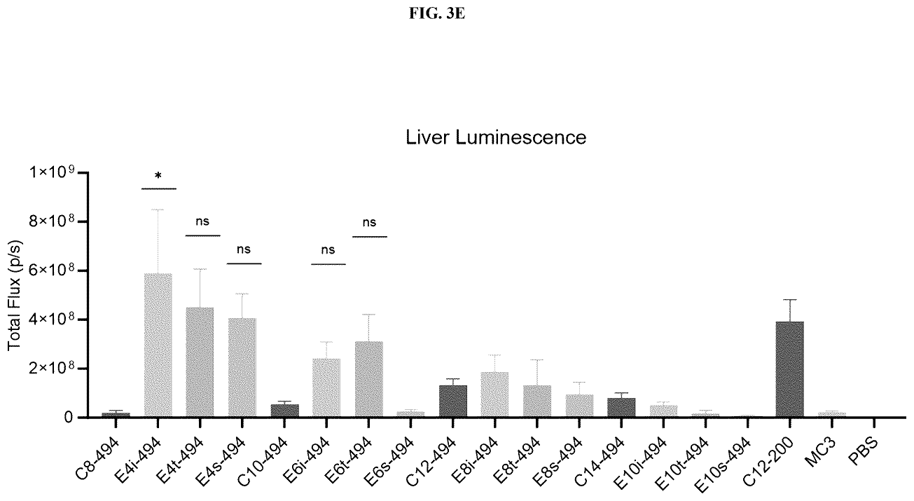

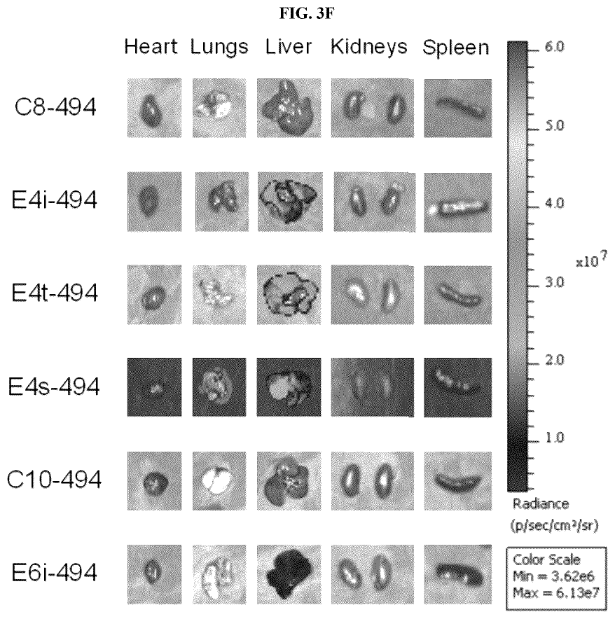

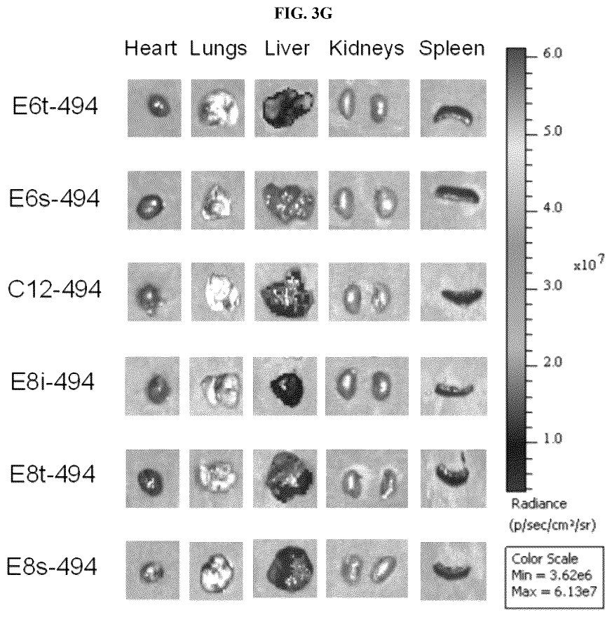

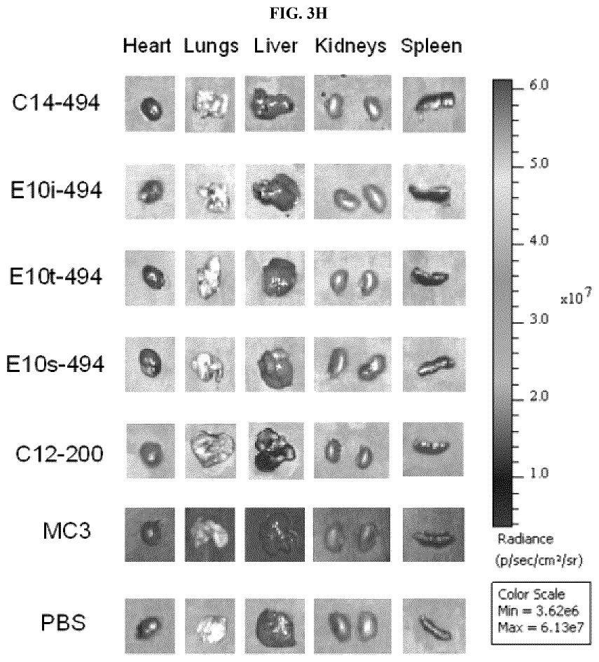

The efficacy of exemplary LNPs of the present disclosure were evaluated in both in vitro and in vivo models. Each LNP was evaluated for transfection efficacy in HeLa cells by incubating the LNPs at a dose of 20 ng of mRNA per 20,000 cells for 24 h. The luminescence results indicated that branching significantly enhanced LNP transfection at the lowest and highest lipid lengths by 10-fold, whereas in the middle lengths, branching induced no significant change or greatly decreased transfection (FIG. 3A). When examined for toxicity using a CellTiter-Glo assay, seven LNPs demonstrated statistically significant toxicity; however, none of the LNPs had viability below 80% o (FIG. 3B).

To verify the consistency of this trend, the LNPs were administered intravenously into C57BL/6J mice at a dose of 0.1 mg/kg. DLin-MC3-DMA (MC3), an FDA-approved LNP formulation for the delivery of siRNA, and C₁₂-200, a more potent LNP for mRNA delivery with a similar polyamine core as 494, were also administered to serve as positive controls. After 12 h, full body and organ luminescence were obtained (FIGS. 3C-3D). Contrary to the in vitro results, all LNPs with branched ILs (branched LNPs) performed as well as or better than the corresponding LNPs with linear ILs (linear LNPs) for total RNA delivery and liver delivery. For full body delivery, six branched LNPs and only one linear LNP induced luciferase expression as well as C12-200, whereas E4t-494 and E6t-494, and no linear LNPs, outperformed C12-200.

All LNPs transfected the liver preferentially over other organs, and in terms of liver luminescence, six LNPs with branched ILs performed as good as or better than C12-200, with E4i-494 facilitating 1.5-fold greater luminescence (FIGS. 3E-3G). Notably, no linear LNPs achieved statistically equal or greater signal than C12-200. When compared against MC3 and not C₁₂-200, four branched LNPs and no linear LNPs had significantly increased luminescence than MC3.

To further investigate the branched lipid paradigm, it was investigated whether lipid branching can enhance the delivery of a liver-trophic amine core, specifically the 200 core exemplified herein. As such, six of the branched epoxides were coupled with the 200 core to form six new branched ILs comprising the 200 core (FIG. 2B). The successful synthesis of the branched 200 core IL underscores the robust nature of the initial branched epoxide design as it can be applied to many different amine cores. The three branching groups were each evaluated at relative lipid lengths of eight carbons and twelve carbons, although the present disclosure is not limited to such chain lengths, and the corresponding linear versions, C8-200 and C12-200, were utilized as controls. While the C12-200 is considered a standard liver-trophic IL, C8-200 performs much poorer and thus represents an important test as to whether branching can also convert this IL into a potent liver targeting LNP.

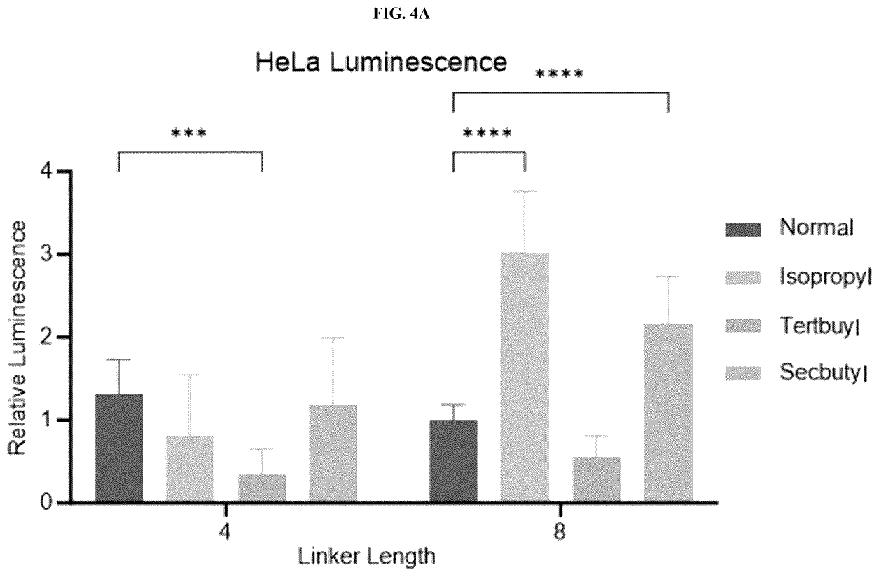

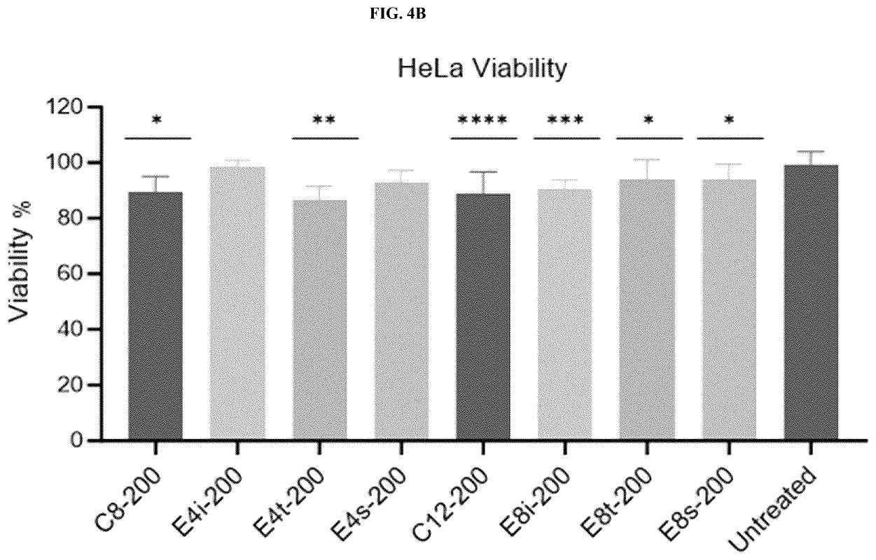

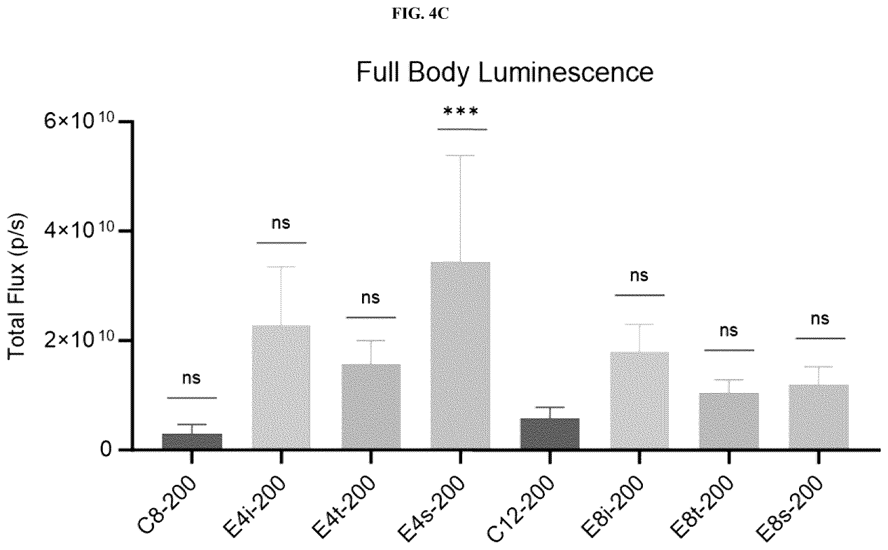

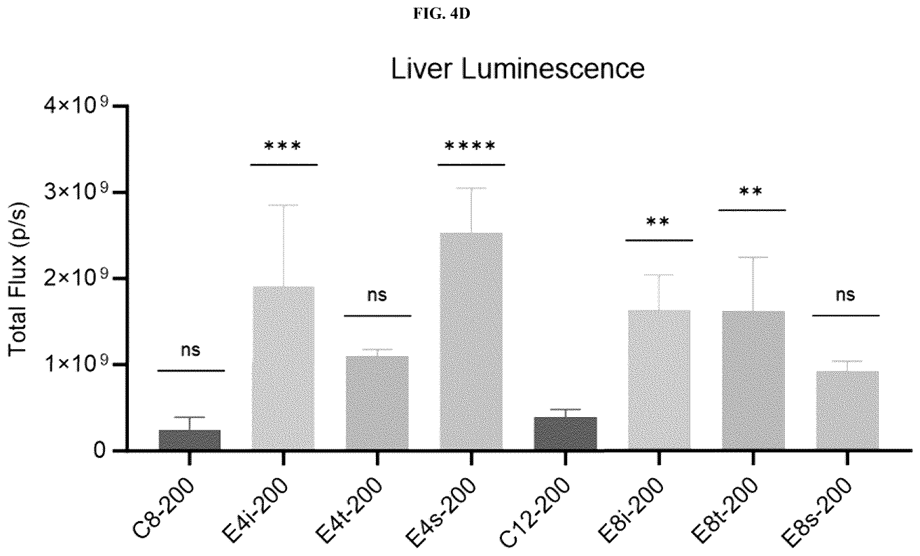

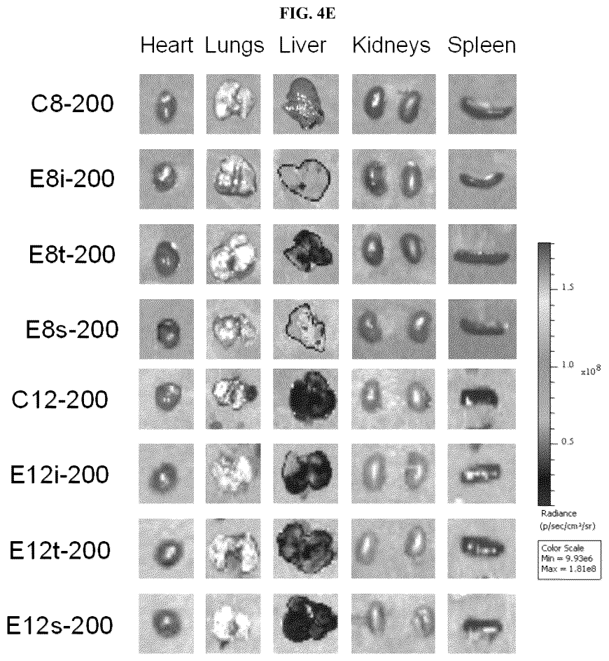

These eight ILs were then formulated into LNPs with fluc mRNA and characterized using the same methodology as described above (FIG. 4A). Akin to the studies with the 494 LNPs, testing in HeLa cells revealed conflicting themes, as C8-200 performed better or the same as the branched equivalents while two of the longer chain branched LNPs strongly outperformed C12-200 (FIG. 4B). None of the LNPs demonstrated toxicity in vitro at the concentration tested for the luciferase assay. Afterwards, the 200 core LNPs were intravenously injected into C57BL/6J mice at 0.1 mg/kg of fluc mRNA. All branched LNPs performed similar to or better than C12-200 for full body luminescence and liver delivery (FIGS. 4C-4E), including the C8-200 branched lipids, with E4s-200 inducing five-fold greater liver luminescence. These results demonstrate that branching at lipid termini enhance liver delivery irrespective of the lipid core, providing an essential design criterium for future ILs.

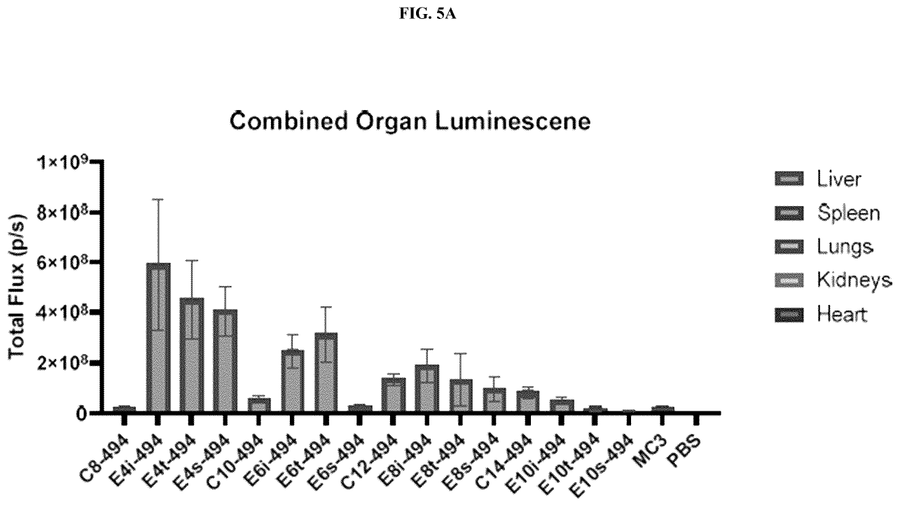

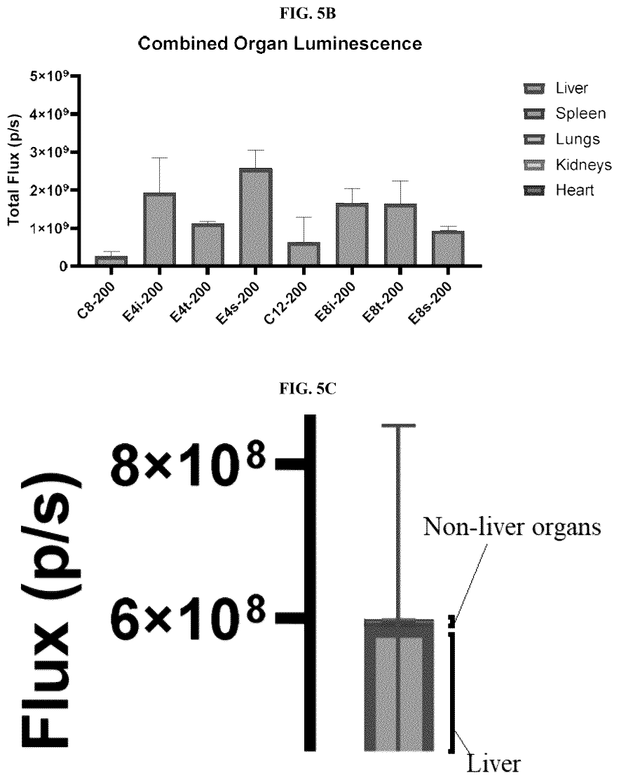

In certain embodiments, the present disclosure provides delivery data comprising luminescence data (e.g., full body, liver, and/or combined organ luminescence data) obtained by administration of exemplary LNPs comprising TriLink Luciferase to Black 6 mice. In certain embodiments, the mice were also administered one or more controls (e.g., LNPs comprising MC3 and/or unbranched ionizable lipids). In certain embodiments, a subset of LNPs comprising an ionizable lipid prepared from the polyamine core 494 were evaluated in this manner, and the data demonstrated enhanced luminescence with several of the LNPs of the present disclosure as compared to LNPs comprising unbranched ionizable lipids, with respect to organ luminescence (FIG. 5A). In certain embodiments, a subset of LNPs comprising an ionizable lipid prepared from the polyamine core 200 were evaluated in this manner, and the data demonstrated enhanced luminescence with several of the LNPs of the present disclosure as compared to LNPs comprising unbranched ionizable lipids, with respect to combined organ luminescence (FIG. 5B).

Further, the experiments provided herein indicate that the LNPs of the present disclosure were selective for distribution to the liver, as compared to the spleen, lungs, kidneys, and/or heart, with little to no luminescence observed in non-liver organs (FIGS. 5A-5D).

### Example 3: Branched LNPs Induce Potent Gene Editing

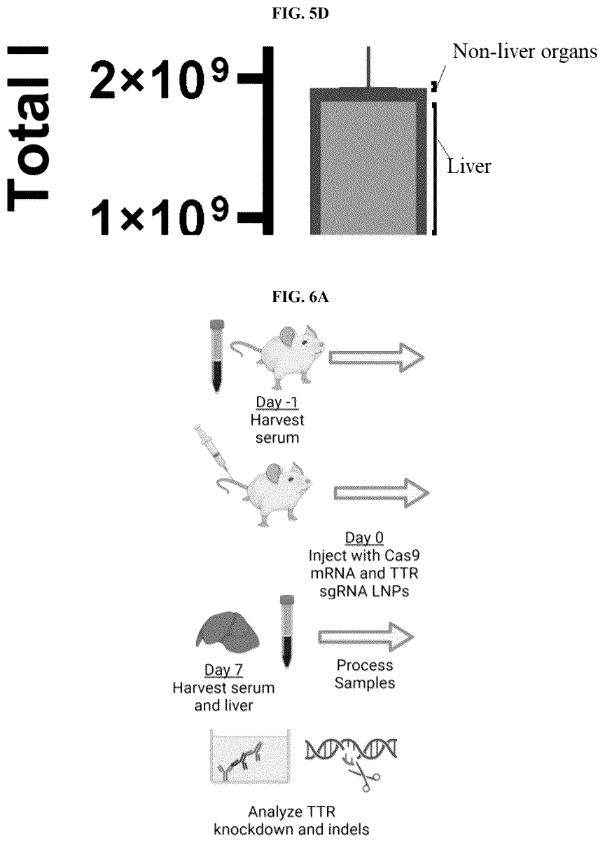

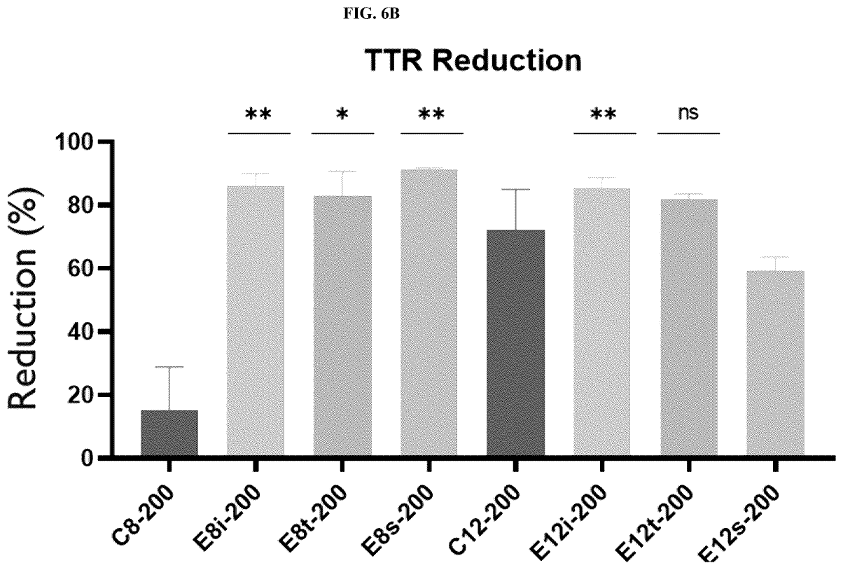

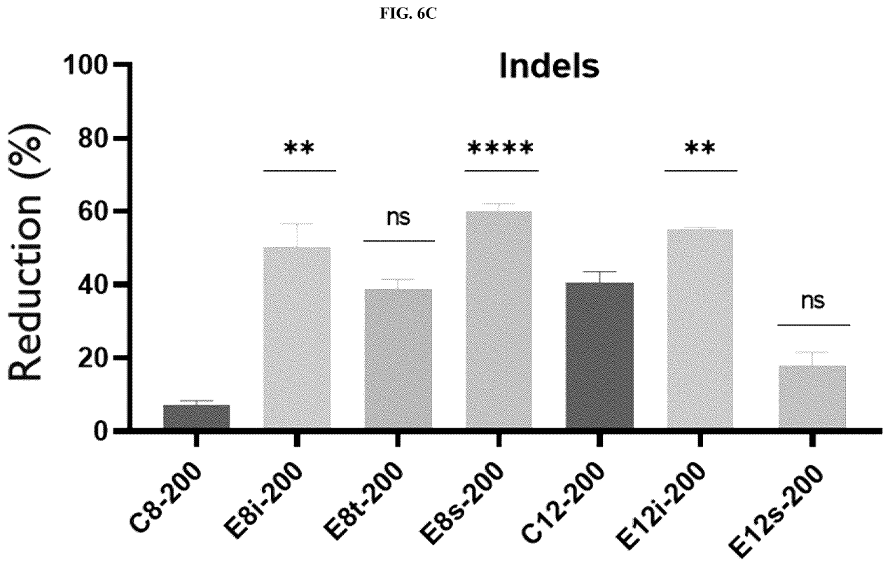

Next, LNP delivery was evaluated in a clinically-relevant murine model for transthyretin (TTR)-mediated amyloidosis. TTR is a protein that when overexpressed in the liver, will develop into amyloid fibrils that can induce restrictive cardiomyopathy and heart failure. LNP-based siRNA therapies, such as the MC3-based ONPATTRO® have been utilized to silence TTR translation in the liver; however, this can require multiple transfusions as siRNA has a limited therapeutic window. Instead, delivering gene editing machinery can offer a single dose therapy that would permanently reduce TTR to safe levels. As such, the eight 200 core LNPs were reformulated to encapsulate Cas9 mRNA and single guide RNA (sgRNA) targeting TTR. C57BL/6J mice were then injected with 0.3 mg/kg or 1.0 mg/kg of combined Cas9 and TTR sgRNA. After seven days, blood was removed, serum was isolated, and the levels of TTR protein were evaluated compared to serum isolated from the same mice 24 h before the study (FIG. 6A).

The mice were sacrificed following blood withdrawal, and the livers were removed, underwent DNA isolation, and analyzed for indels using next-generation sequencing (NGS). While the C12-200 LNP reduced TTR levels by 70%, several of the LNPs with branched ILs reduced TTR levels by 80-90% (FIG. 6B). Additionally, these latter LNPs induced 50-60% indels, while the C12-200 LNP only induced 40% indels (FIG. 6C). These results demonstrate that lipid branching can enhance delivery regardless of the mRNA, showcasing the universality of this design.

To evaluate toxicity, the mice were reinjected with the same formulation. At 12 h, blood was withdrawn and after 24 h, the major organs were removed, stained for H&E, and analyzed for toxic markers via an independent expert. Upon analyzing the blood for liver damage markers, E4i-200, E4s-200, and E8t-200 had significantly higher AST levels compared to the control group, while C12-200 do not. However, for ALT, E4s-200, C12-200, E8i-200, and E8t-200 all had greater levels than PBS.

### Example 4: Branched ILs Facilitate Greater Endosomal Escape

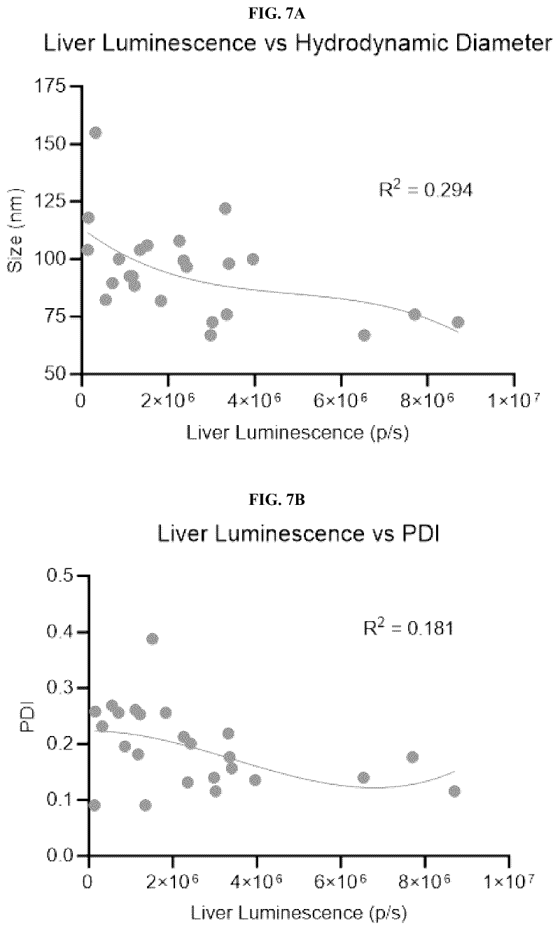

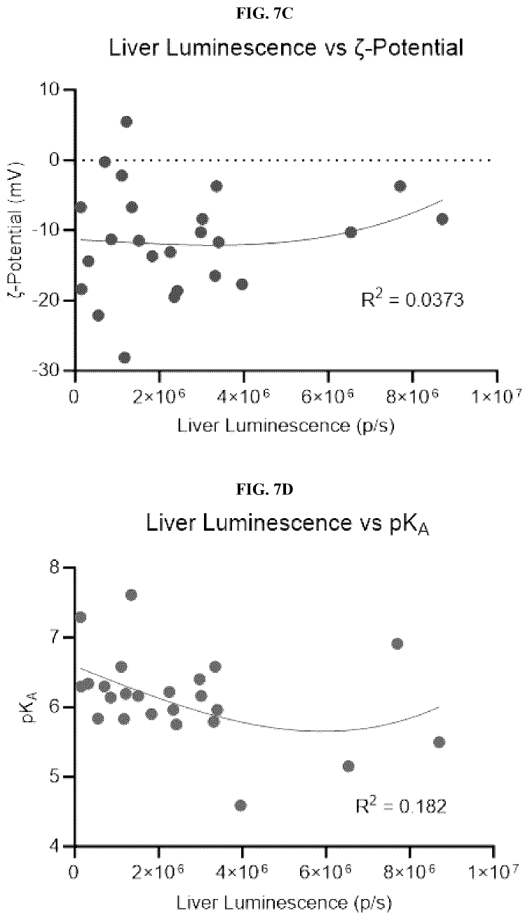

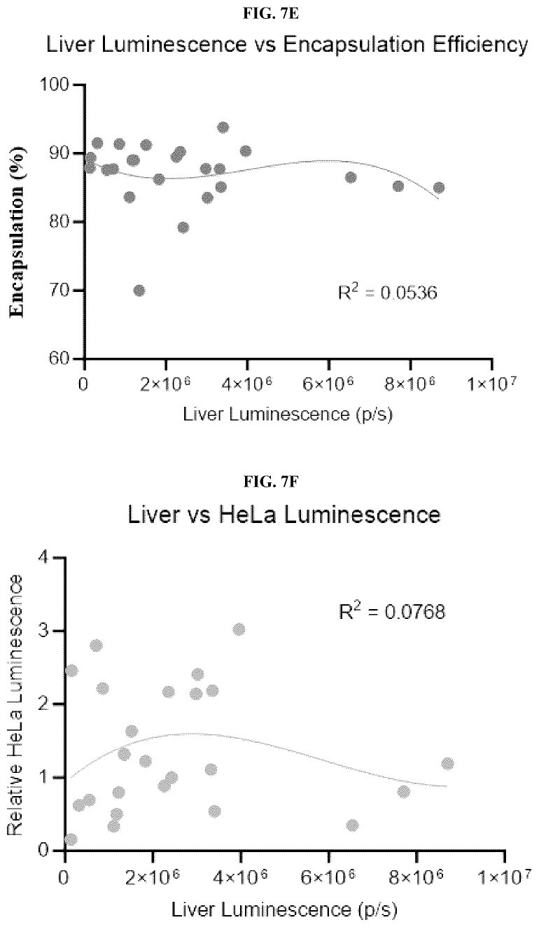

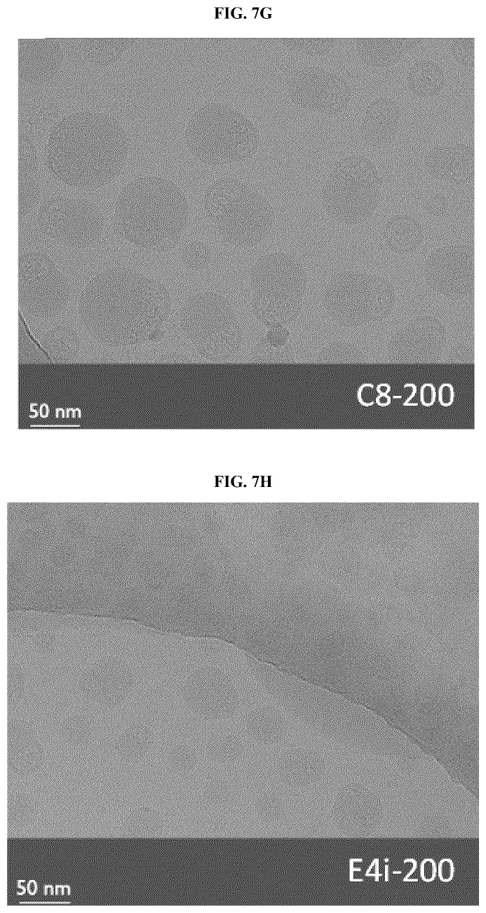

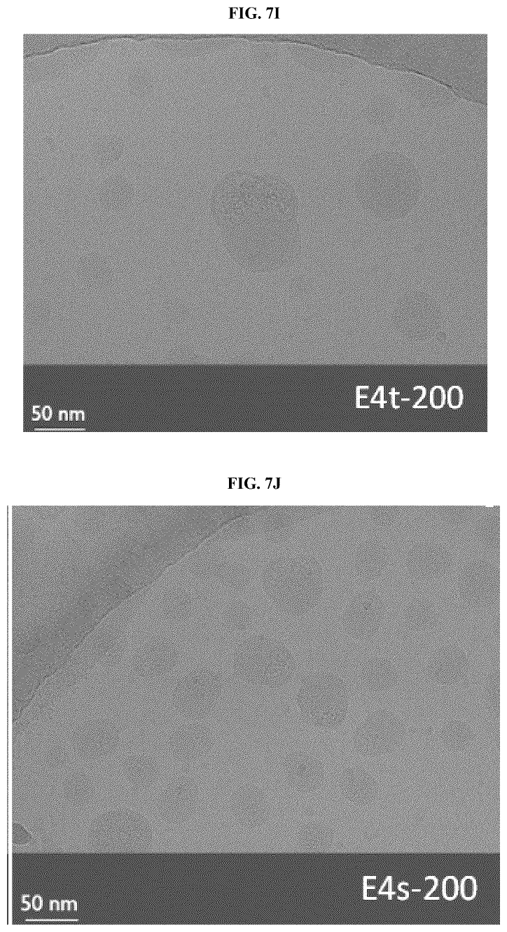

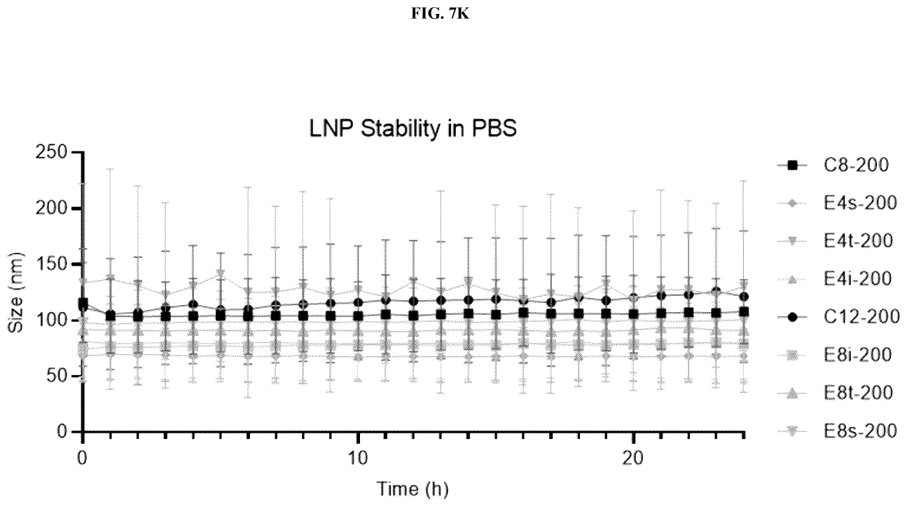

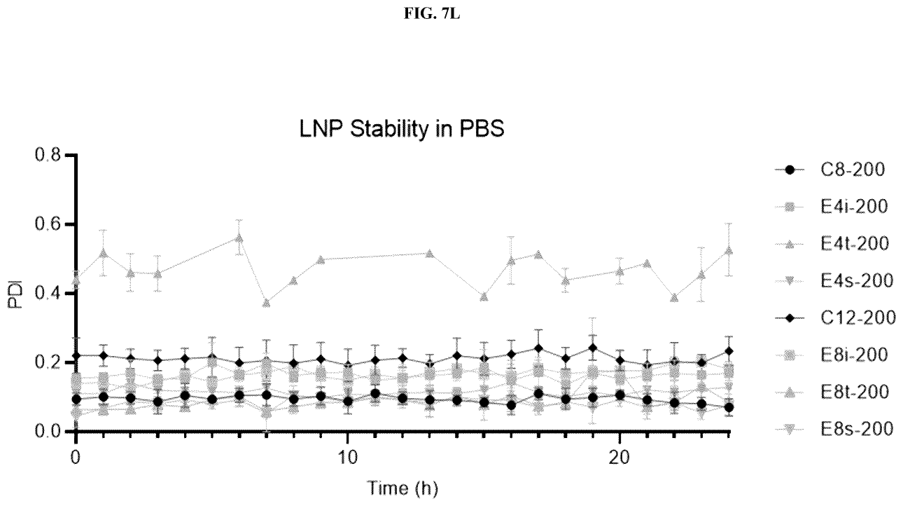

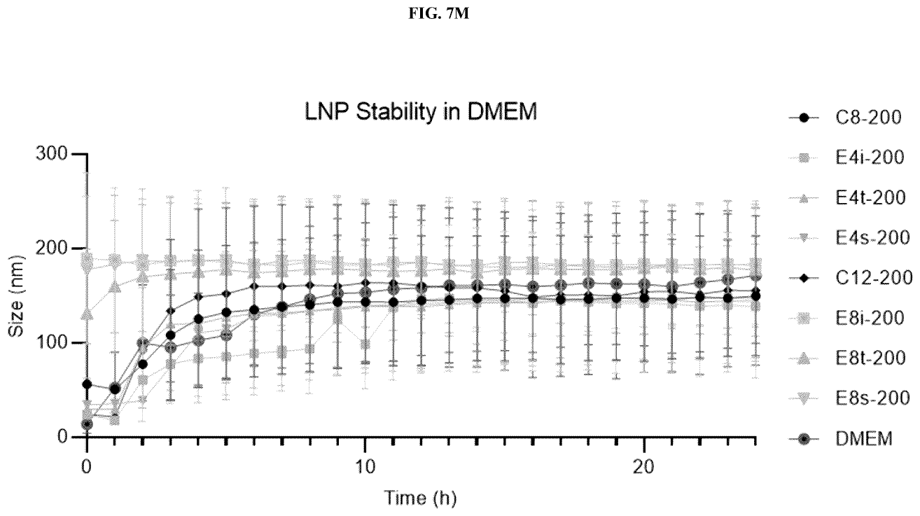

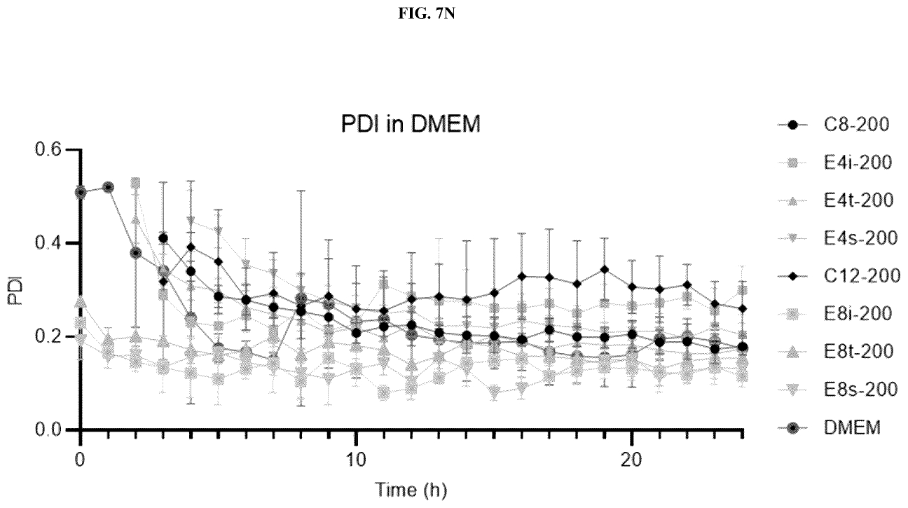

To determine the mechanism of the increased efficacy of branched LNPs, several possible explanations were evaluated. Initially, it was evaluated whether lipid branching results in LNPs with optimal physicochemical parameters, by correlating the resulting liver luminescence of each LNP from the studies with fluc mRNA with the particle's hydrodynamic diameter, PDI, ζ-potential, and pKₐ (FIGS. 7A-7E). The success in liver delivery was further correlated with HeLa cell transfection (FIG. 7F). Intriguingly, after fitting the data with a cubic least squares regression, no parameter was predictive of successful liver delivery. Moreover, HeLa cell transfection was also a very poor predictor of in vivo delivery. Following this study, it was analyzed whether the morphology of branched LNPs differed significantly from linear LNPs; however, upon analyzing the structure via cryo-TEM, all LNPs had a similar egg-shaped morphology (FIGS. 7G-7J). This suggests that this form of lipid branching may have minimal impact on the global arrangement of LNPs, although this does not exclude the possibility of finer changes in bilayer and monolayer arrangement.

The stability of the LNPs was next evaluated by incubating exemplary LNPs in PBS and Dulbecco's Modified Eagle Medium (DMEM) substituted with 10% fetal bovine serum (FBS) at 37° C. The size and PDI of the LNPs were measured every hour for 24 h by dynamic light scattering immediately after incubation (FIGS. 7K-7N). Similar to the characterization results, no significant destabilization was observed in either PBS or DMEM, and in the latter, all LNPs formed a similar sized protein corona.

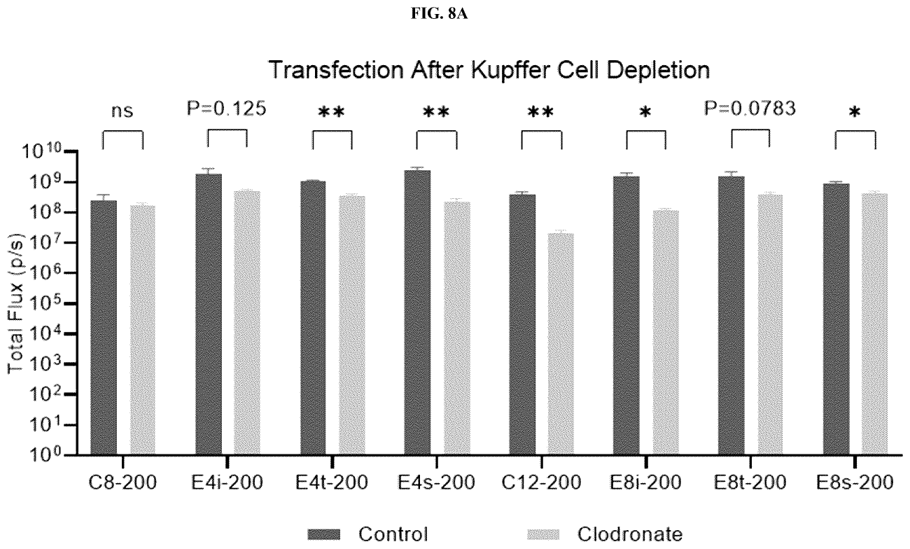

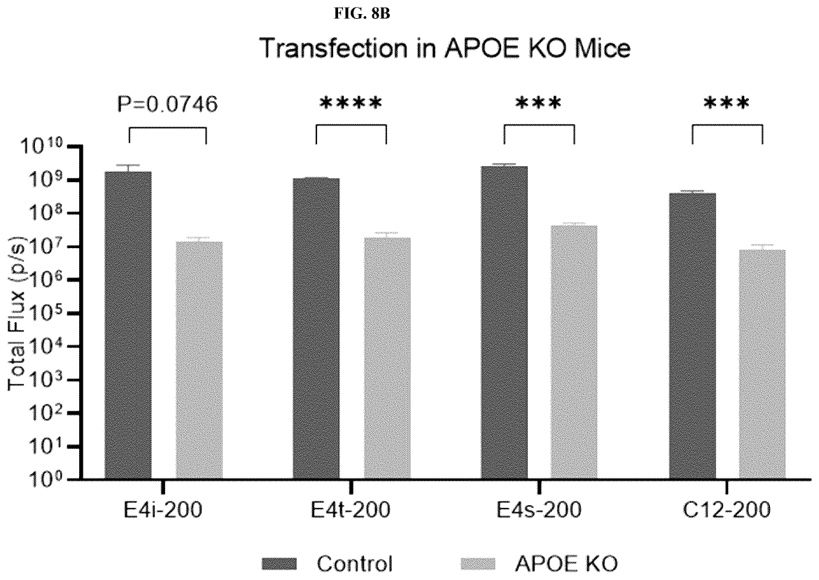

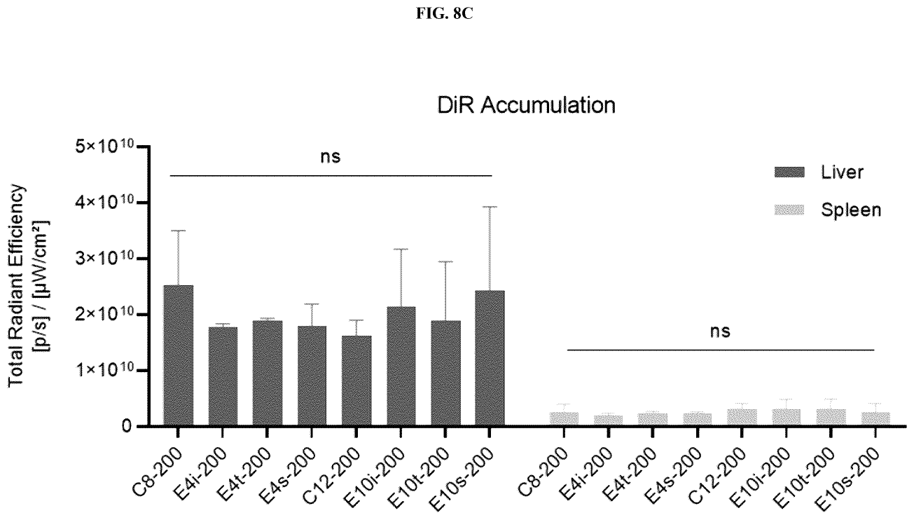

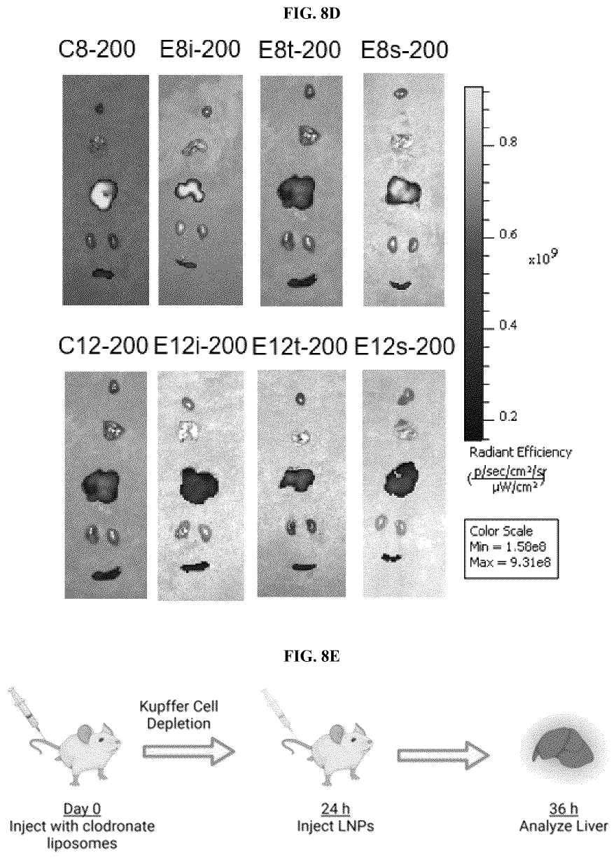

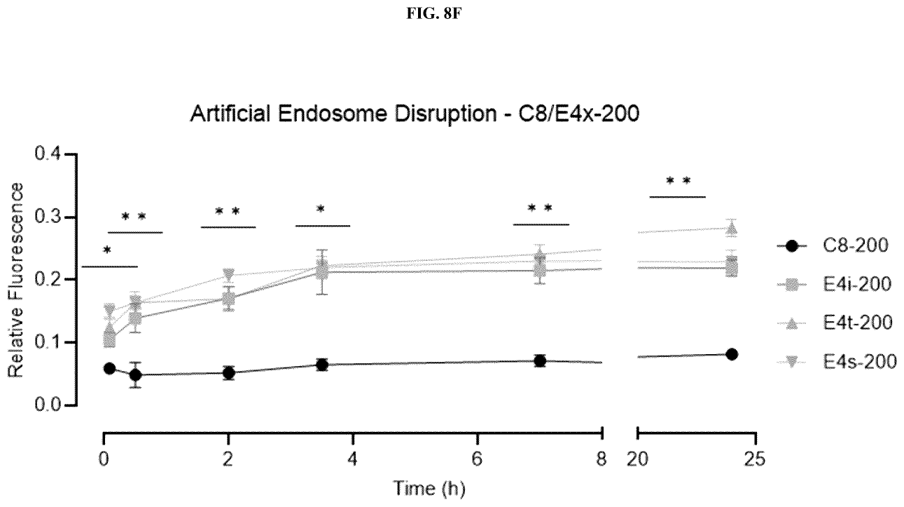

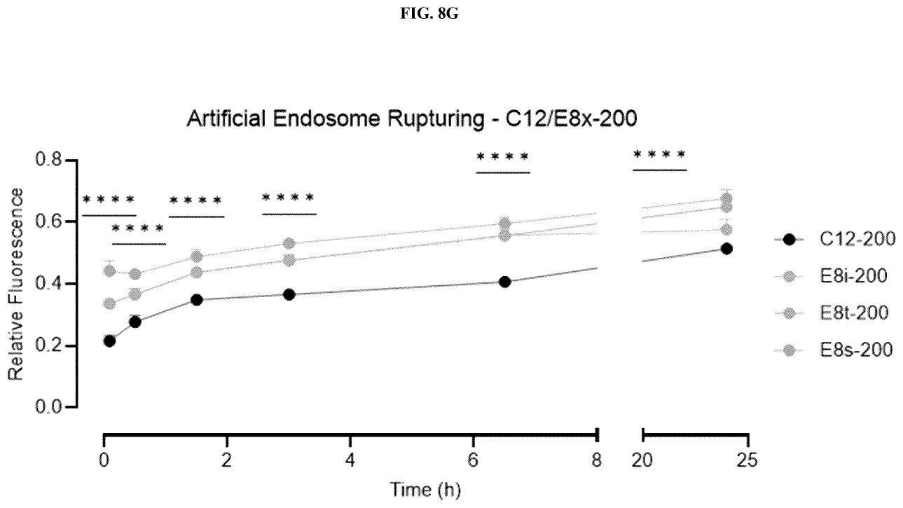

Since particle physicochemical characteristics, morphology, and stability seemed to be unaffected by lipid branching, the role of liver trafficking and uptake was investigated. Several studies have demonstrated that IL structure can impact the specific liver cells that take up LNPs. For example, Kupffer cells are liver macrophages that more favorably capture certain types LNPs, which can result in different liver biodistributions. As such, C57BL/6J mice were injected with clodronate liposomes, which deplete macrophages, and after 24 h, reinjected the mice with the eight 200-core LNPs encapsulating fluc mRNA (FIG. 8E). After a total of 36 h, liver luminescence was imaged, revealing that all LNPs except for C8-200 and E4i-200 had significant decreases in luminescence (FIG. 8A).

These data suggest that branching is likely not a factor in the prevalence of macrophage uptake. Still, C8-200 and E4i-200, the two LNPs with ILs of the smallest lipid lengths, transfect macrophages at a lower frequency compared to non-macrophage cells; although, a larger library would need to be screened to validate this trend.

Next, the role of protein corona and liver interactions was investigated by injecting E4i-200, E4t-200, E4s-200, and C12-200 fluc LNPs into apolipoprotein E (APOE) knockout mice at 0.1 mg/kg. Upon measuring liver luminescence after 12 h, all four LNPs had dramatic decreases of 100-1000 fold luminescence compared to wild type mice (FIG. 8B). This suggests that both branched and unbranched LNPs target the liver via an APOE-mediated mechanism, as has been demonstrated with many other liver-trophic LNPs.

Next, particle accumulation in the liver was analyzed. LNPs containing fluc were reformulated with 1 mol % DiR, a lipophilic carbocyanine near IR fluorescent dye. The fluorescent LNPs were injected into C57BL/6J mice at 0.1 mg/kg of fluc mRNA, and the major organs were dissected and imaged for fluorescence after 12 h. Here, fluorescence is a marker of LNP accumulation. While mRNA transfection and translation had occurred exclusively in the liver, LNP fluorescence was observed in the liver and spleen (FIGS. 8C-8D). More interestingly, all LNPs showed similar fluorescence in both organs.

Without wishing to be bound by any theory, if all eight LNPs are accumulating in the liver at relatively the same amounts, but the branched LNPs are facilitating greater mRNA translation, it was hypothesized that this result is likely due to the branched ILs allowing a higher number of mRNA molecules to escape the endosome and enter the cytosol. While there are other reports of certain IL structural moieties influencing endosomal escape, the specifical role of terminal branching remains unknown in the art.

To verify this result, the interactions of the eight LNPs with artificial endosomes was studied, which is a validated strategy to study the role of endosomal escape. Exemplary endosomes were formulated via thin film hydration of DOPE, 18:1 Δ9-Cis phosphocholine (DOPC), 18:1 phospho-L-serine (DOPS), NBD-conjugated 18:1 phosphoethanolamine (NBD-PE), and Lissamine-Rhodamine-B-conjugated 18:1 phosphoethanolamine (Liss-Rhod-PE) at molar ratios of 48:25:25:1:1, respectively. The latter two lipids are a FRET pair that was used to monitor the integrity of the endosome. Increases in the fluorescence of the donor fluorophore corresponds to endosomal disruption as the FRET pair is separated. In this study, each LNP was diluted 6× in pH 5.5 buffer to represent endosomal conditions and mixed with the artificial endosomes. At various timepoints, the fluorescence of the donor fluorophore was measured. The branched LNPs induced 2-to-3-fold greater fluorescence than the linear LNPs for both the C8-200 (FIG. 8F) and C12-200 (FIG. 8G) groups, supporting the idea that lipid branching facilitates more pronounced endosomal escape and the linear versions.

### Example 5: Oral Cancer Tumor Suppressor Therapy

The present disclosure further provides exemplary data relating to the use of the LNPs described herein for treating and/or ameliorating oral cancer tumors. Exemplary LNPs of the present disclosure utilized LNPs comprising ionizable lipids prepared from the 494 polyamine core, including C8-C14-, E4i-E10i-, E4t-E10t-, and E4s-E10s-494 ionizable lipids, whereas C12-200 was utilized as a positive control.

In the present study, CAL-27 cells were used as a model to evaluate the utility of LNPs of the present disclosure for delivery of certain mRNA cargo. CAL-27 cells are a human HPV-negative tongue squamous cell carcinoma which are considered a gold standard cell line for studies of HPV-negative oral cancer.

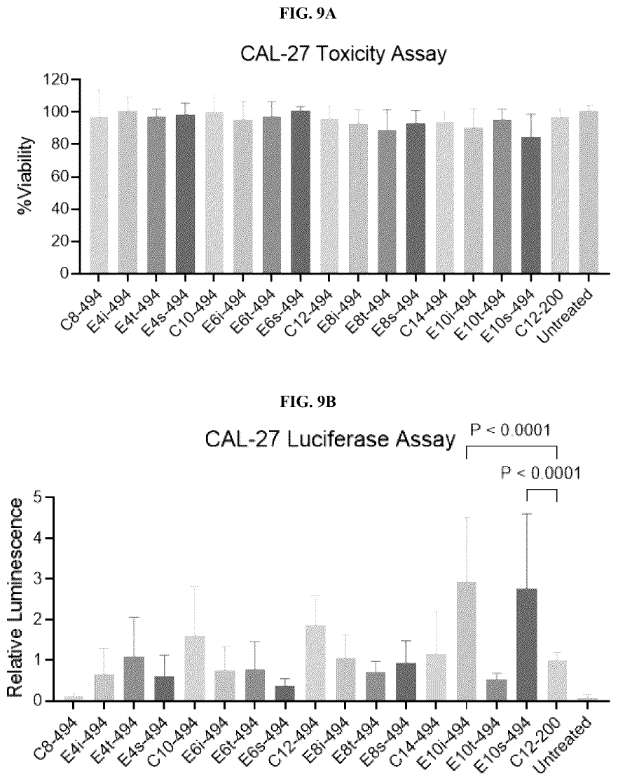

Exemplary LNPs were incubated with luciferase mRNA, and the resultant LNPs were subsequently administered to the cells at a dose of 20 ng mRNA per 20,000 cells. After 24 h, a luciferase assay (FIG. 9A) and cell viability assay (FIG. 9B) were performed to analyze luciferase expression and toxicity, respectively. Luciferase assay data was normalized to C12-200. Several of the evaluated LNPs outperformed the C12-200 LNP (e.g., E10i-494 and E10s-494), while two LNPs comprising branched ionizable lipids significantly outperformed the C12-200 LNP.

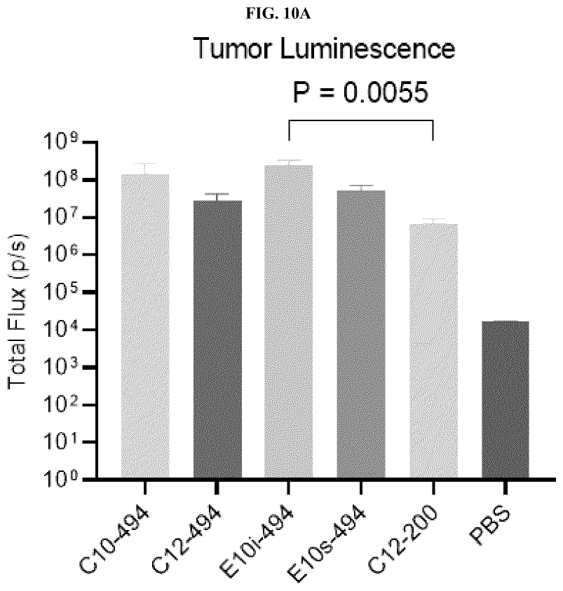

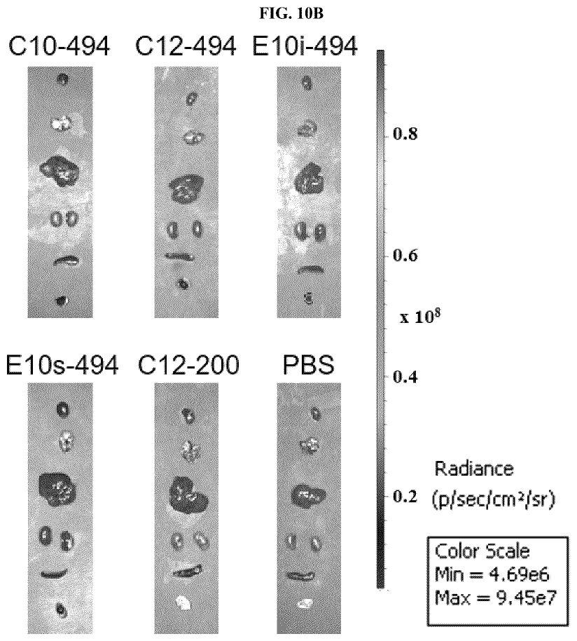

The present disclosure further describes the use of a mouse tumor model, wherein Nu/J mice were inoculated on the right flank with CAL-27 cells, and tumors were permitted to grow for 2 weeks. Subsequently, five exemplary LNPs encapsulating luciferase mRNA, including two LNPs comprising branched ionizable lipids and three LNPs comprising linear ionizable lipids, were injected intratumorally at a dose of 0.1 mg/kg, and certain mice were administered PBS as a control. The mice were sacrificed, the major organs and tumor were harvested, and imaged for fluorescence (FIGS. 10A-10B). The results provided herein indicate that the top performing LNP (i.e., E10i-494), which comprises a branched ionizable lipid, had over 10-fold greater signal than was observed for the LNP comprising C12-200.

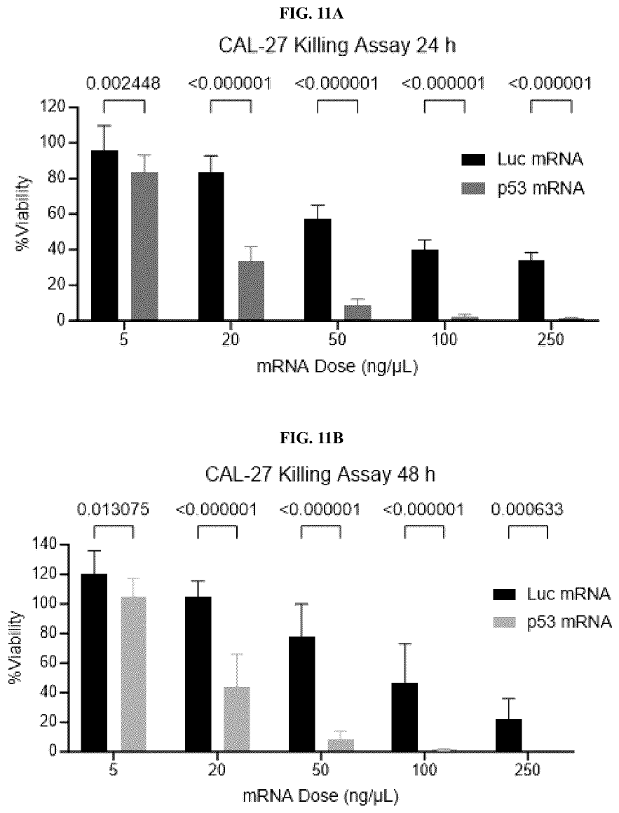

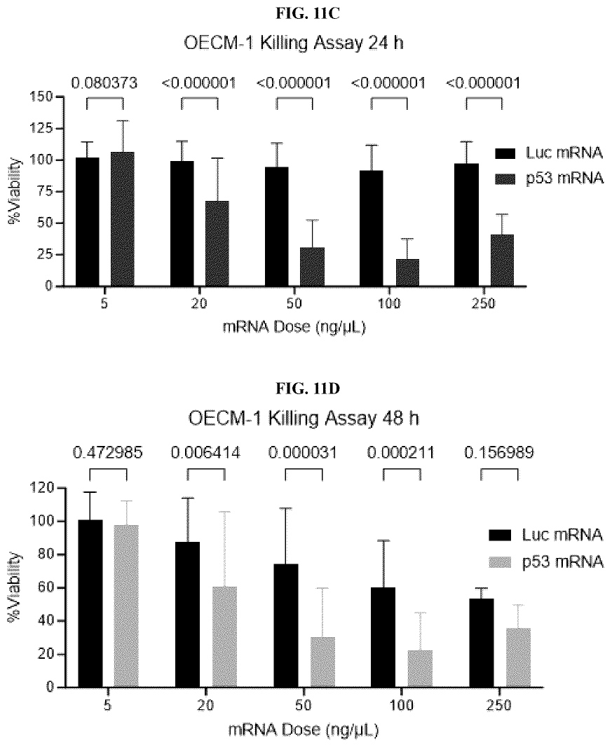

The present disclosure further describes the use of the LNPs of the present disclosure in viability assays (i.e., killing assays) utilizing CAL-27 and OECM-1 cell lines, wherein OECM-1 cells represent a HPV-negative squamous cell carcinoma cell line. Exemplary LNPs utilized in the studies described herein comprise p53 cargo. In one aspect, p53 represents an ideal target for LNP-mediated suppressor therapy, as over 70% of oral squamous cell carcinomas have mutated or downregulated p53. A range of mRNA doses were utilized (i.e., 5, 20, 50, 100, and 250 ng/μL) at varying time intervals (i.e., 24 h or 48 h) and overall viability was measured (FIGS. 11A-11D). Low viability (i.e., cell killing) was observed with administration of exemplary LNPs comprising p53 mRNA at least partially encapsulated therein after both 24 and 48 h.

### Example 6: Stem Cell Reprogramming

The present disclosure further provides exemplary data relating to the use of the LNPs described herein for reprogramming stem cells (e.g., induced pluripotent stem cells (iPSCs)). iPSCs are derived from skin or blood cells that have been reprogrammed back into an embryonic-like pluripotent state that enables the development of a source of any type of human cell needed for therapeutic purposes. For example, an iPSC can be differentiated into a blood cell to create new blood free of cancer cells for a leukemia patient.

Transfection of iPSCs with mRNA cargo permits expression of certain biomolecules of interest and may permit differentiation to a cell line of interest (e.g., neuron and/or muscle cells), as desired. In the experiments described herein, iPSCs were transfected with mRNA at least partially encapsulated in exemplary LNPs of the present disclosure, said LNPs comprising ionizable lipids prepared from the 494 and 200 polyamine cores, whereas LNPs comprising C12-200 were utilized as a positive control.

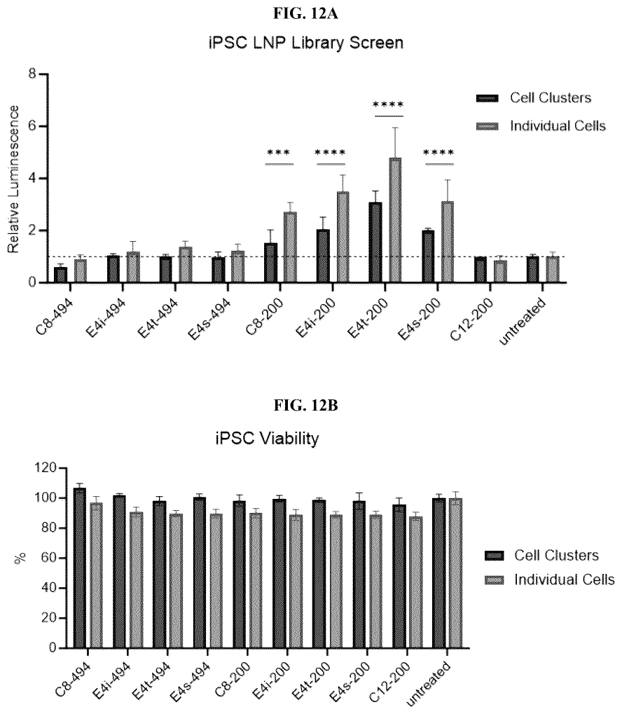

An initial potency screen of exemplary LNPs was performed with LNPs of the present disclosure comprising luciferase mRNA (20 ng/15,000 cells) and luciferase expression (FIG. 12A) and viability were measured (FIG. 12B). Comparable viability was observed with each exemplary LNP, whereas certain LNPs (i.e., E4i-200, E4t-200, and E4s-200) exhibited high relative fluorescence.

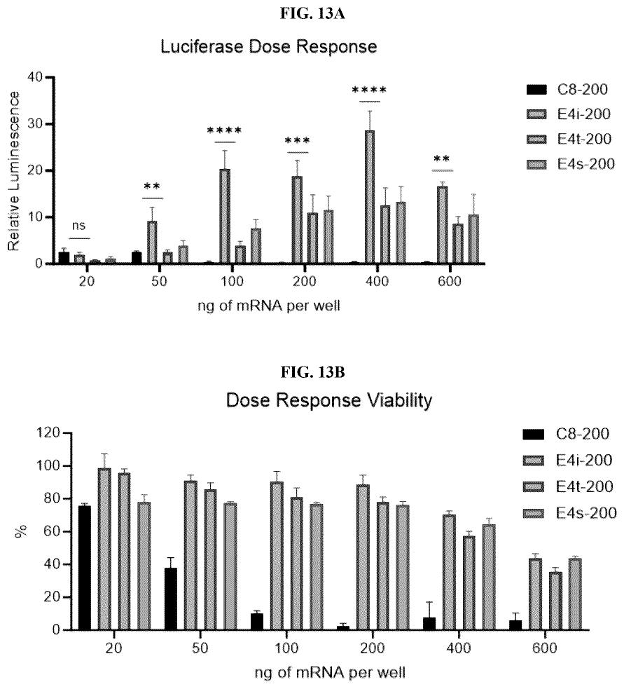

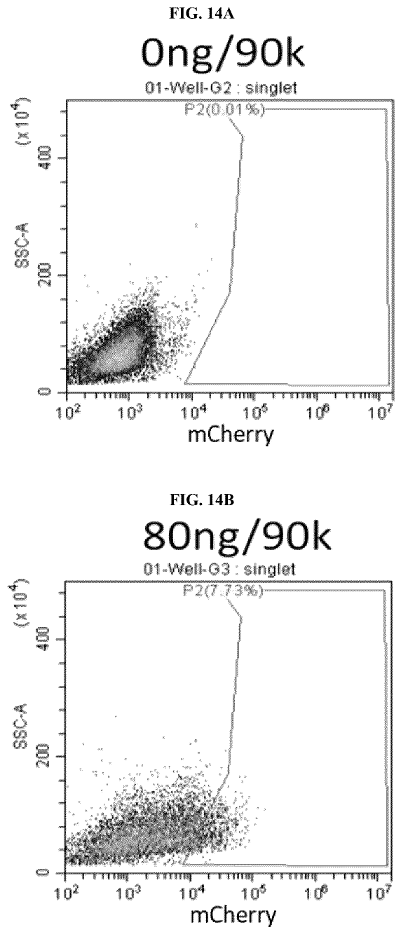

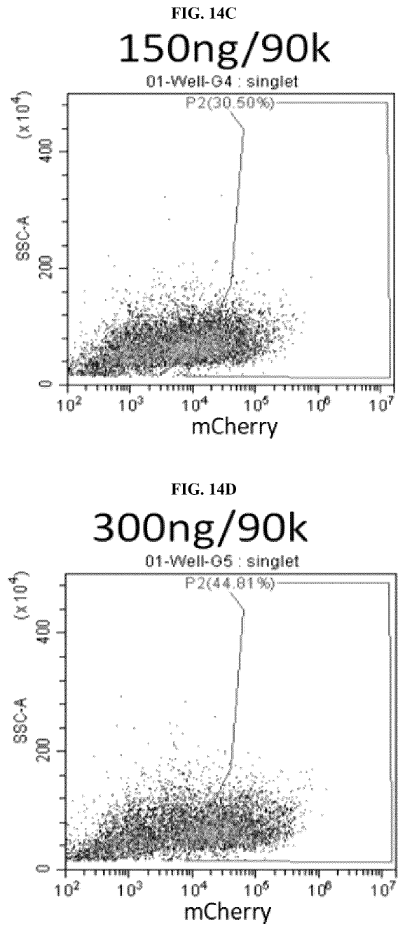

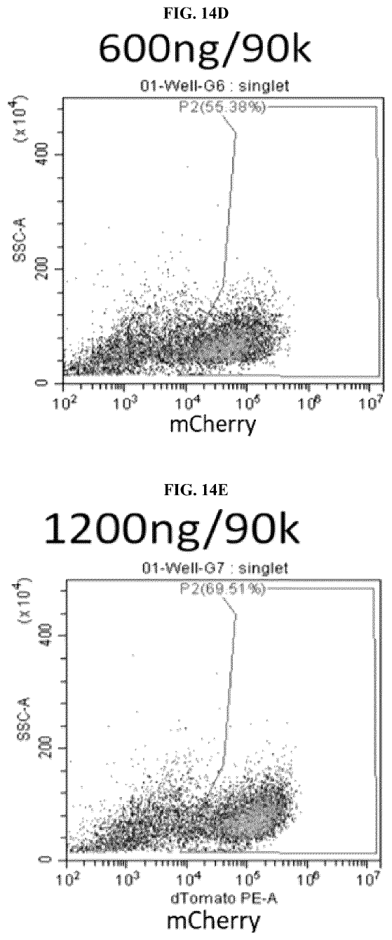

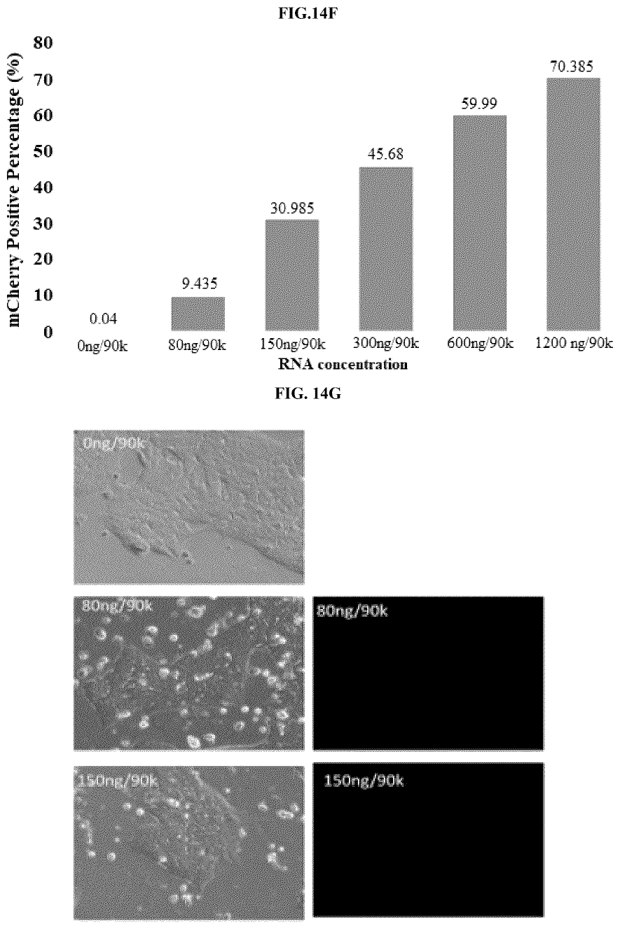

Dose response luciferase luminescence (FIG. 13A) and viability (FIG. 13B) experiments were performed with top performing LNPs identified in the screening studies. LNP E4i-200 was identified as a top performer, with respect to relative luminescence. E4i-200 LNP was reformulated with mCherry. iPSC-SV20 cells were plated in 24-well plates overnight and subsequently treated with E4i-LNP comprising mCherry. Flow cytometry was performed to measure the percentage of cells that were successfully transfected and cells were imaged by fluorescence microscopy to visualize the mCherry signal. (FIGS. 14A-14H).

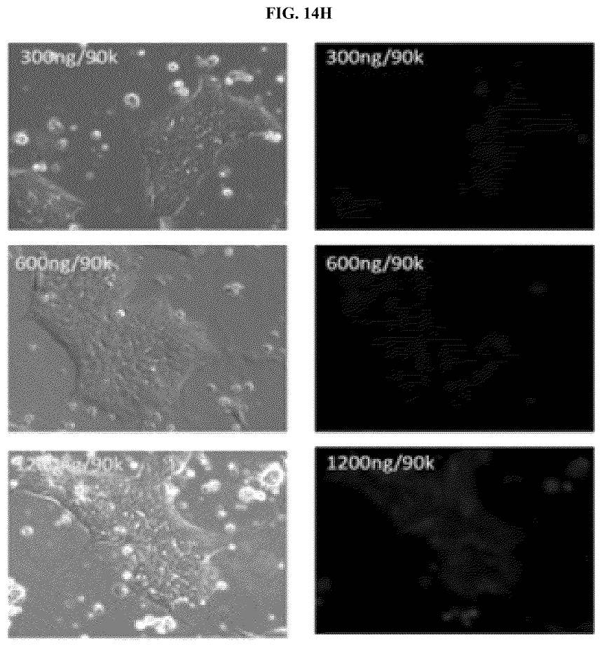

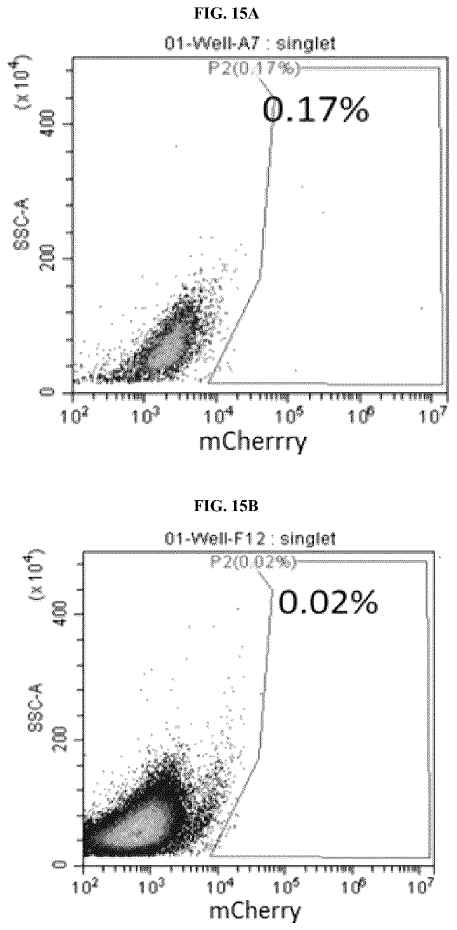

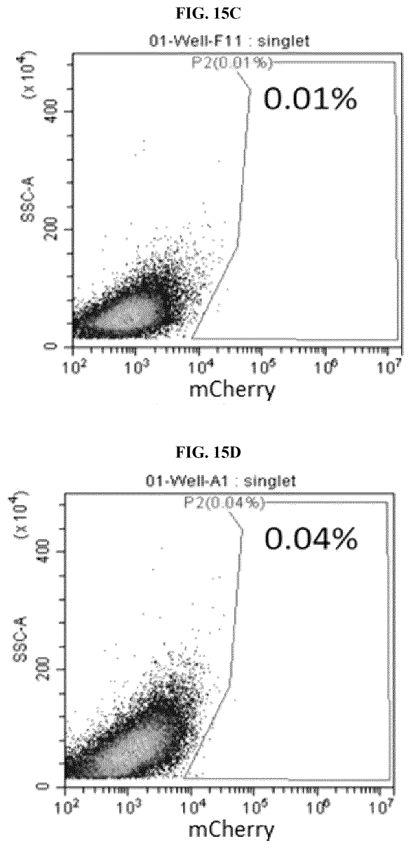

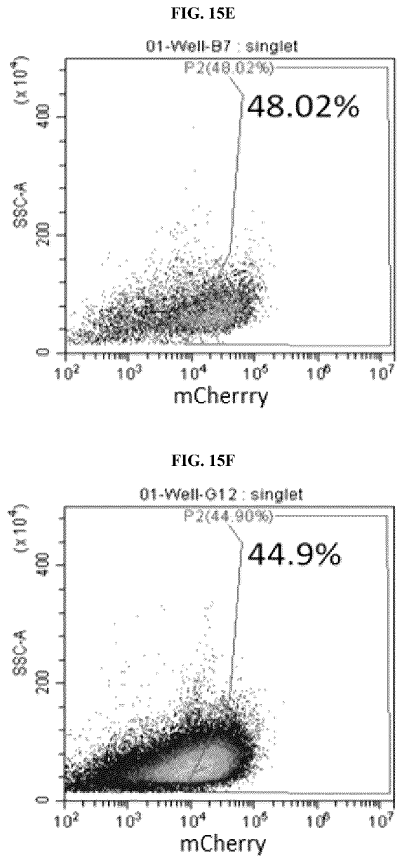

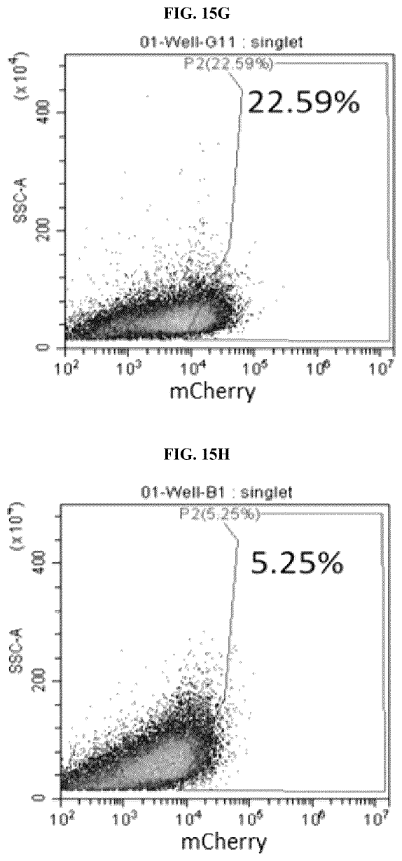

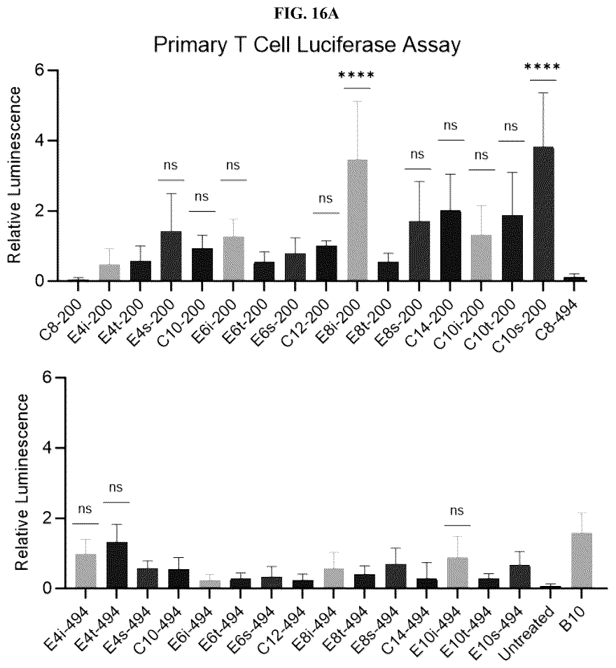

A time-point study was performed, wherein iPSCs transfected with mCherry (or controls) were harvested after 24 h (FIGS. 15A and 15E), 48 h (FIGS. 15B and 15F), 72 h (FIGS. 15C and 15G), and 96 h (FIGS. 15D and 15H). The results provided herein demonstrate that the mCherry signal remained at high intensity for 48 hours, as determined by FACs analysis.

### Example 7: CAR-T Cell and CAR-Natural Killer (NK) Cell Therapy

In another aspect, the present disclosure relates to the use of the LNPs of the present disclosure for use in CAR-T and CAR-NK therapy.

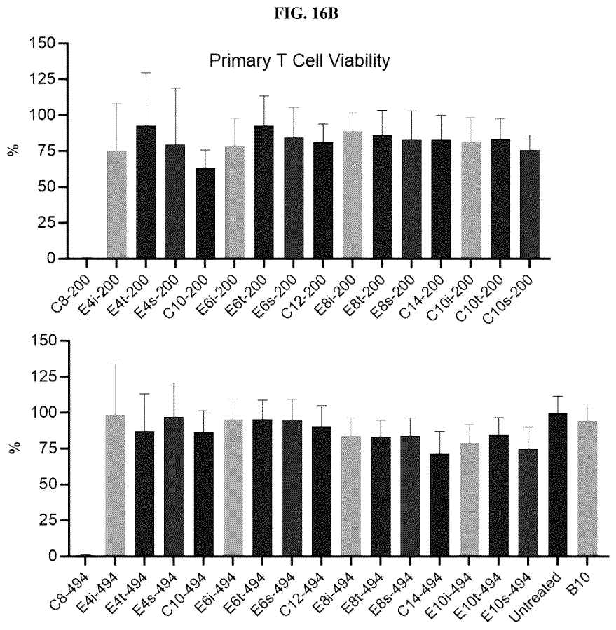

The experiments described herein utilize LNPs comprising ionizable lipids prepared from the 494 and 200 polyamine cores. An initial screen of LNPs was performed utilizing exemplary LNPs comprising luciferase mRNA were incubated in activated primary T cells (1:1 CD4+:CD8+) from health human donors, wherein luciferase was present at a concentration of 200 ng mRNA/60,000 cells. Luciferase expression (FIG. 16A) and cell viability (FIG. 16B) was measured after 24 h, wherein E8i-200 and E10s-200 LNPs were found to outperform the B10 LNP formulation, with minimal toxicity.

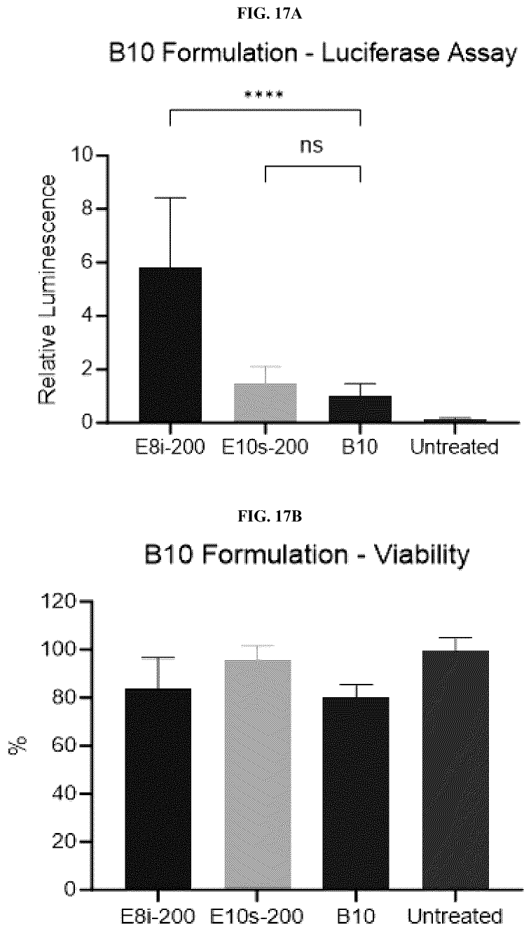

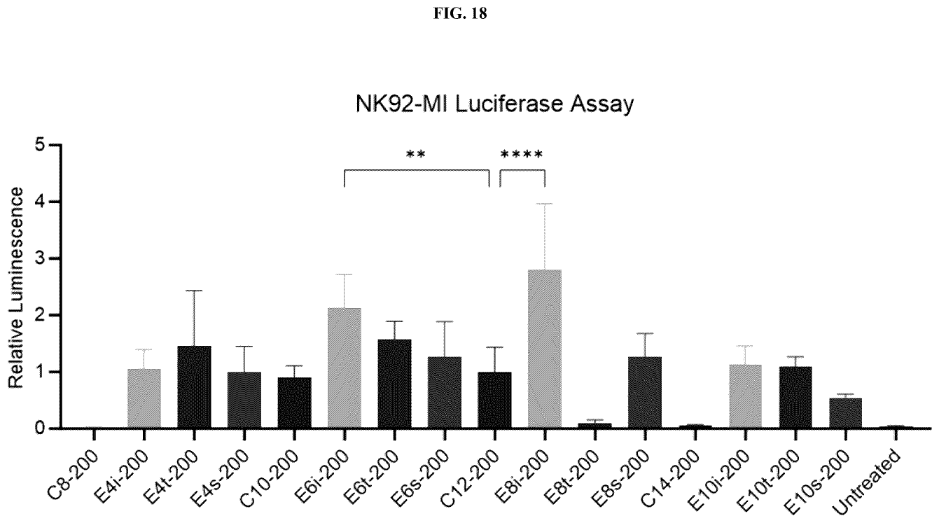

The formulation of LNP components of the top performing LNPs identified in the screen described herein was modified to match the formulation used for the B10 LNP (i.e., ionizable lipid:DOPE:cholesterol:C14PEG2000 of 40:30:25:2.5), and relative luminescence (FIG. 17A) and viability (FIG. 17B) of the exemplary LNPs in primary T cells were measured. LNP E8i-200 was identified as a top performer in this experiment, demonstrating almost 6-fold greater luminescence than B10, with negligible change in viability.

The present disclosure further describes evaluation of exemplary LNPs for transfection of NK-92MI cells (i.e., immortalized human natural killer cells which self-express IL-2). In these experiments, LNPs comprising luciferase mRNA (200 ng mRNA/60,000 cells) were incubated with NK-92MI cells and luciferase expression was measured after 24 h. Certain exemplary LNPs comprising branched ionizable lipids (e.g., E6i-200 and E8i-200) significantly outperformed the LNP comprising C12-200, thereby demonstrating utility of the LNPs of the present disclosure for transfection of immune cells (e.g., T cells and NK cells) (FIG. 18).

Sequence Listing
SEQ ID NO: 1
ususasCAGCCACGUCUACAGCAGUUUUAGAgcuagaaauagcAAGUUAA
AAUAAGGCUAGUCCGUUAUCAacuugaaaaaguggcaccgagucggugcu
sususu
SEQ ID NO: 2
CGGTTTACTCTGACCCATTTC
SEQ ID NO: 3
GGGCTTTCTACAAGCTTACC

### Enumerated Embodiments

The following exemplary embodiments are provided, the numbering of which is not to be construed as designating levels of importance:

Embodiment 1 provides an ionizable lipid compound of Formula (I), or a salt, solvate, stereoisomer, or isotopologue thereof:

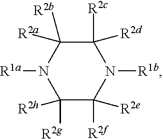

wherein:
R^{1a} and R^{1b} are each independently

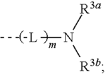

R^{2a}, R^{2b}, R^{2c}, R^{2d}, R^{2e}, R^{2f}, R^{2g}, and R^{2h} are each independently selected from the group consisting of H, optionally substituted C₁-C₁₂ alkyl, optionally substituted C₂-C₁₂ heteroalkyl, optionally substituted C₃-C₈ cycloalkyl, optionally substituted C₂-C₈ heterocycloalkyl, optionally substituted C₂-C₁₂ alkenyl, optionally substituted C₂-C₁₂ alkynyl, optionally substituted C₆-C₁₀ aryl, and optionally substituted C₂-C₁₀ heteroaryl;
each occurrence of R^{3a}, R^{3b}, and R^{3c} is independently selected from the group consisting of H,

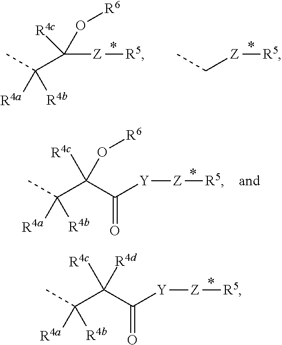

wherein no more than one of R^{3a}, R^{3b}, and R^{3c} is H;
each occurrence of R^{4a}, R^{4b}, R^{4c}, and R^{4d}, if present, is independently selected from the group consisting of optionally substituted C₁-C₁₂ alkyl, halogen, CN, and NO₂;
each occurrence of R⁵ is independently selected from the group consisting of optionally substituted C₁-C₃ alkyl, optionally substituted C₃-C₁₂ cycloalkyl, optionally substituted C₆-C₁₀ aryl, and optionally substituted C₂-C₁₀ heteroaryl;
each occurrence of R⁶ is independently selected from the group consisting of H, optionally substituted C₁-C₆ alkyl, optionally substituted C₃-C₁₂ cycloalkyl, C₂-C₁₂ heterocycloalkyl, optionally substituted C₆-C₁₂ aralkyl, optionally substituted C₆-C₁₂ aryl, optionally substituted C₂-C₁₂ heteroaryl, C(═O)R^{a}, C(═O)OR^{a}, and C(═O)N(R^{a})(R^{b});
each occurrence of L is independently selected from the group consisting of a bond, -(optionally substituted C₁-C₁₂ alkylenyl)-X—, -(optionally substituted C₂-C₁₂ alkenylenyl)-X—, -(optionally substituted C₁-C₁₂ alkynylenyl)-X—, -(optionally substituted C₁-C₁₂ heteroalkylenyl)-X—, optionally substituted C₃-C₈ cycloalkylenyl, and optionally substituted C₂-C₈ heterocyloalkylenyl;
each occurrence of X, if present, is independently selected from the group consisting of a bond, —N(R^{3c})—, and —O—;
each occurrence of Y, if present, is independently selected from the group consisting of a bond, —N(R^{a})—, and —O—;
each occurrence of Z is C₁-C₂₄ alkylenyl,
   wherein the C₁-C₂₄ alkylenyl in each occurrence of Z is independently substituted with at least one substituent selected from the group consisting of C₁-C₁₂ alkyl and C₁-C₁₂ haloalkyl, and
   wherein the C₁-C₂₄ alkylenyl in each occurrence of Z is independently optionally further substituted;
each occurrence of R^{a} and R^{b} is independently selected from the group consisting of H, optionally substituted C₁-C₆ alkyl, optionally substituted C₃-C₈ cycloalkyl, optionally substituted C₁-C₆ haloalkyl, optionally substituted C₆-C₁₂ aralkyl, optionally substituted phenyl, optionally substituted C₂-C₈ heteroaryl, C(═O)R^{c}, C(═O)OR^{c}, and C(═O)N(R)(R^{d});
each occurrence of R^{c} and R^{d} is independently selected from the group consisting of H, optionally substituted C₁-C₆ alkyl, optionally substituted C₃-C₈ cycloalkyl, optionally substituted C₁-C₆ haloalkyl, optionally substituted C₆-C₁₂ aralkyl, optionally substituted phenyl, and optionally substituted C₂-C₈ heteroaryl; and
each occurrence of m is independently an integer selected from the group consisting of 1, 2, 3, and 4.

Embodiment 2 provides the compound of Embodiment 1, wherein at least one of the following applies:
(a) at least one selected from the group consisting of R^{2a}, R^{2b}, R^{2c}, R^{2d}, R^{2e}, R^{2f}, R^{2g}, and R^{2h} is H;
(b) at least two selected from the group consisting of R^{2a}, R^{2b}, R^{2c}, R^{2d}, R^{2e}, R^{2f}, R^{2g}, and R^{2h} are H;
(c) at least three selected from the group consisting of R^{2a}, R^{2b}, R^{2c}, R^{2d}, R^{2e}, R^{2f}, R^{2g}, and R^{2h} are H;
(d) at least four selected from the group consisting of R^{2a}, R^{2b}, R^{2c}, R^{2d}, R^{2e}, R^{2f}, R^{2g}, and R^{2h} are H;
(e) at least five selected from the group consisting of R^{2a}, R^{2b}, R^{2c}, R^{2d}, R^{2e}, R^{2f}, R^{2g}, and R^{2h} are H;
(f) at least six selected from the group consisting of R^{2a}, R^{2b}, R^{2c}, R^{2d}, R^{2e}, R^{2f}, R^{2g}, and R^{2h} are H;
(g) at least seven selected from the group consisting of R^{2a}, R^{2b}, R^{2c}, R^{2d}, R^{2e}, R^{2f}, R^{2g}, and R^{2h} are H; and
(h) each of R^{2a}, R^{2b}, R^{2c}, R^{2d}, R^{2e}, R^{2f}, R^{2g}, and R^{2h} are H.

Embodiment 3 provides the compound of Embodiment 1 or 2, wherein each occurrence of L is independently selected from the group consisting of L is independently selected from the group consisting of —(CH₂)₁₋₁₀—, —(CH₂)₂₋₁₀NR^{3c}—, —(CH₂)₂₋₁₀₀—, —(CH₂)₁₋₃—CH(OR^{a})—(CH₂)₁₋₃—, piperazinylenyl, and cyclohexylenyl.

Embodiment 4 provides the compound of any one of Embodiments 1-3, wherein each occurrence of L is independently selected from the group consisting of —CH₂—, —(CH₂)₂—, —(CH₂)₃—, —(CH₂)₁₀—, —(CH₂)₂₀—, —(CH₂)₃₀—, —CH₂CH(OR^{a})CH₂—, —(CH₂)₂NR^{3c}—,

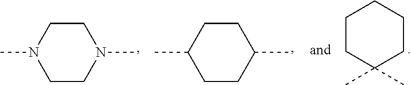

Embodiment 5 provides the compound of any one of Embodiments 1-4, wherein the compound of Formula (I) is selected from the group consisting of:

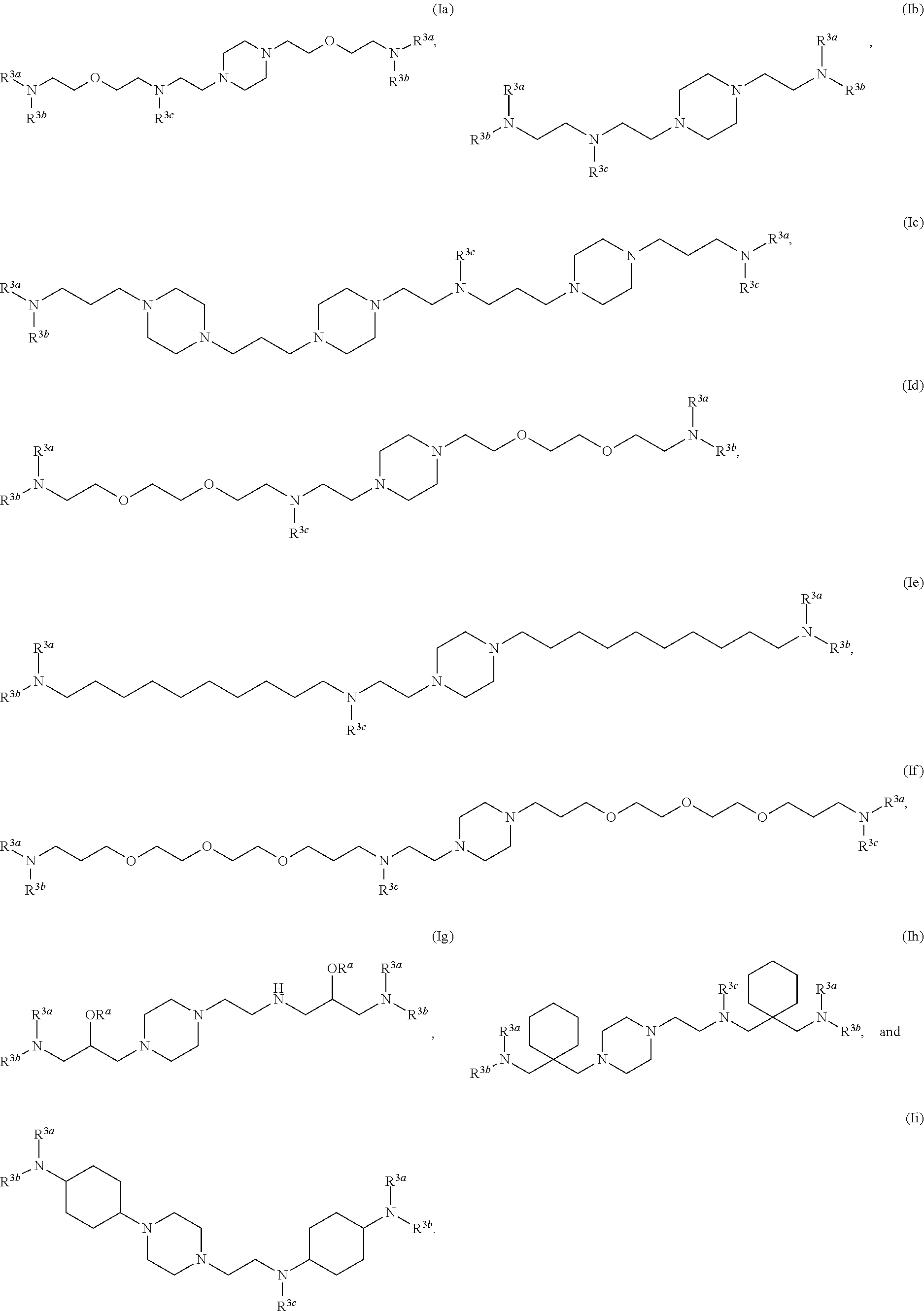

Embodiment 6 provides the compound of any one of Embodiments 1-5, wherein each occurrence of R^{4a}, R^{4b}, R^{4c}, and R^{4d} is independently H.

Embodiment 7 provides the compound of any one of Embodiments 1-6, wherein each occurrence of R⁵ is independently methyl.

Embodiment 8 provides the compound of any one of Embodiments 1-7, wherein each occurrence of R⁶ is independently H.

Embodiment 9 provides the compound of any one of Embodiments 1-8, wherein each occurrence of Z is independently:

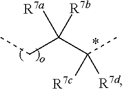

wherein:
each occurrence of R^{7a}, R^{7b}, R^{7c}, and R^{7d} is independently selected from the group consisting of H, C₁-C₆ alkyl, and C₁-C₆ haloalkyl,
   wherein at least one of R^{7a}, R^{7b}, R^{7c}, and R^{7d} is not H; and
each occurrence of o is an integer selected from the group consisting of 1, 2, 3, 4, 5, 6, 7, 8, 9, 10, 11, and 12.

Embodiment 10 provides the compound of Embodiment 9, wherein each occurrence of R^{7a}, R^{7b}, R^{7c}, and R^{7d} is selected from the group consisting of H and methyl.

Embodiment 11 provides the compound of any one of Embodiments 1-10, wherein each occurrence of Z is independently selected from the group consisting of —(CH₂)₄₋₁₀—CH(CH₃)—*, —(CH₂)₄₋₁₀—C(CH₃)₂—*, and —(CH₂)₄₋₁₀—CH(CH₃)—CH₂—*.

Embodiment 12 provides the compound of any one of Embodiments 1-11, wherein each occurrence of R^{3a}, R^{3b}, and R^{3c} is independently selected from the group consisting of

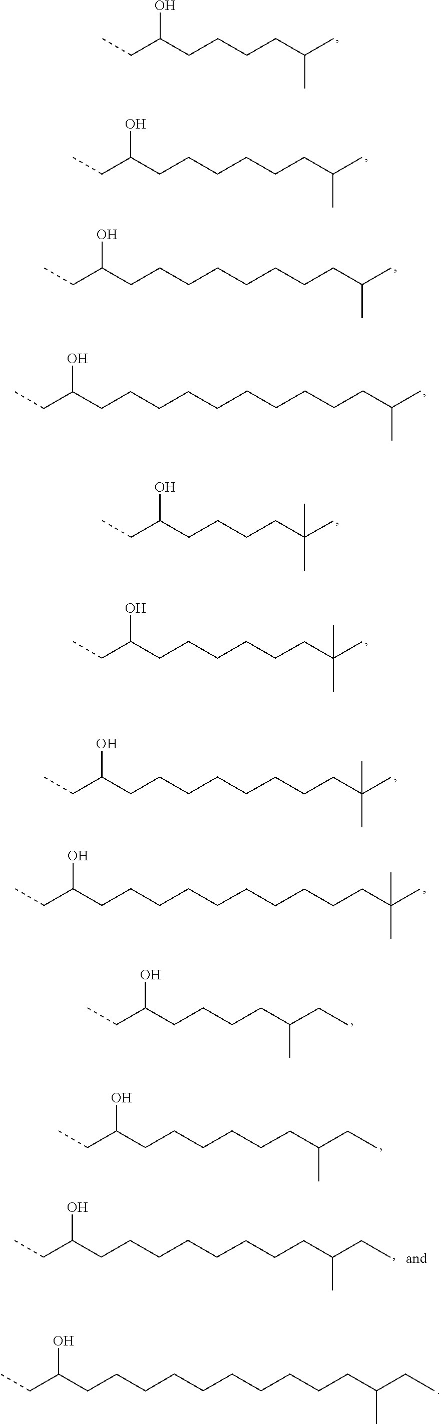

Embodiment 13 provides the compound of any one of Embodiments 1-12, wherein each occurrence of optionally substituted alkyl, optionally substituted heteroalkyl, optionally substituted cycloalkyl, optionally substituted heterocycloalkyl, optionally substituted alkenyl, optionally substituted alkynyl, optionally substituted aryl, optionally substituted heteroaryl, optionally substituted aralkyl, optionally substituted alkylenyl, optionally substituted alkenylenyl, optionally substituted alkynylenyl, optionally substituted heteroalkylenyl, optionally substituted cycloalkylenyl, and optionally substituted heterocycloalkylenyl, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OH, N(R′)(R″), C(═O)R′, C(═O)OR′, OC(═O)OR′, C(═O)N(R′)(R″), S(═O)₂N(R′)(R″), N(R′)C(═O)R″, N(R′)S(═O)₂R″, C₂-C₈ heteroaryl, and phenyl optionally substituted with at least one halogen, wherein each occurrence of R′ and R″ is independently selected from the group consisting of H, C₁-C₆ alkyl, C₃-C₈ cycloalkyl, C₁-C₆ haloalkyl, benzyl, and phenyl.

Embodiment 14 provides the compound of any one of Embodiments 1-13, which is selected from the group consisting of:

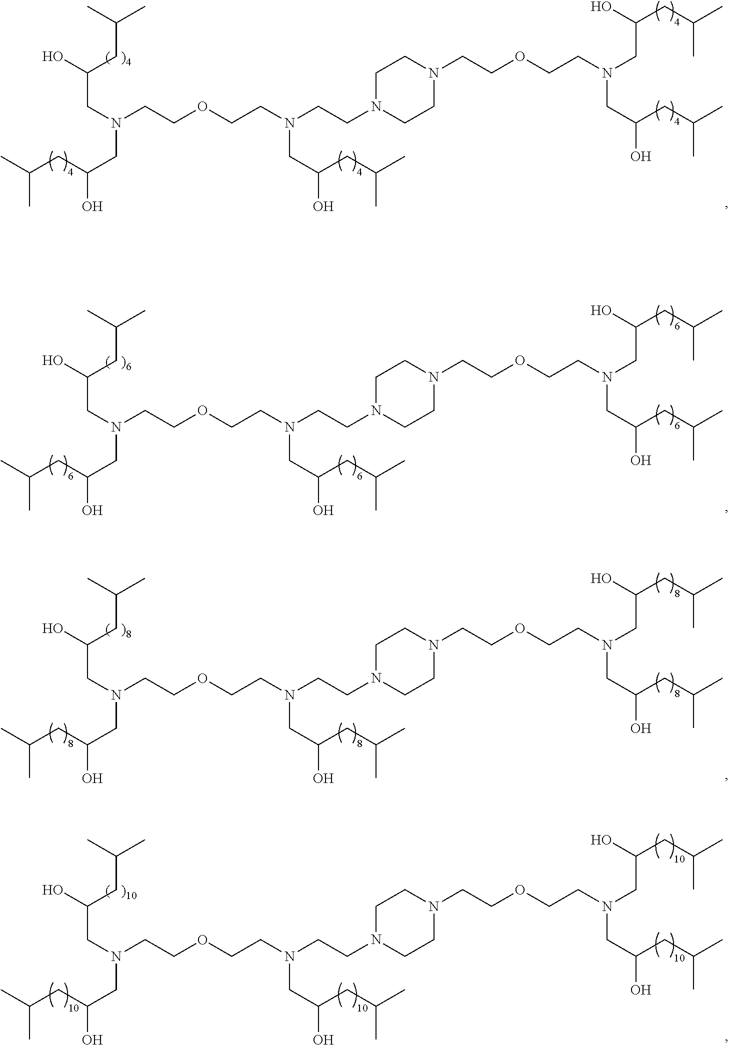

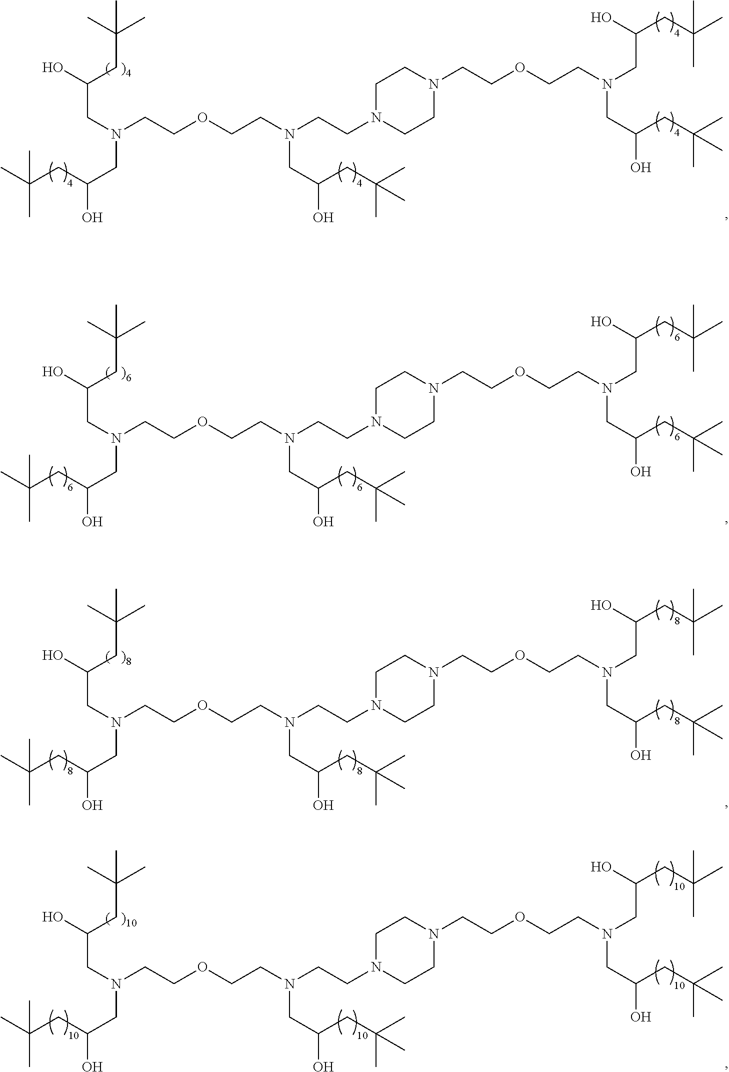

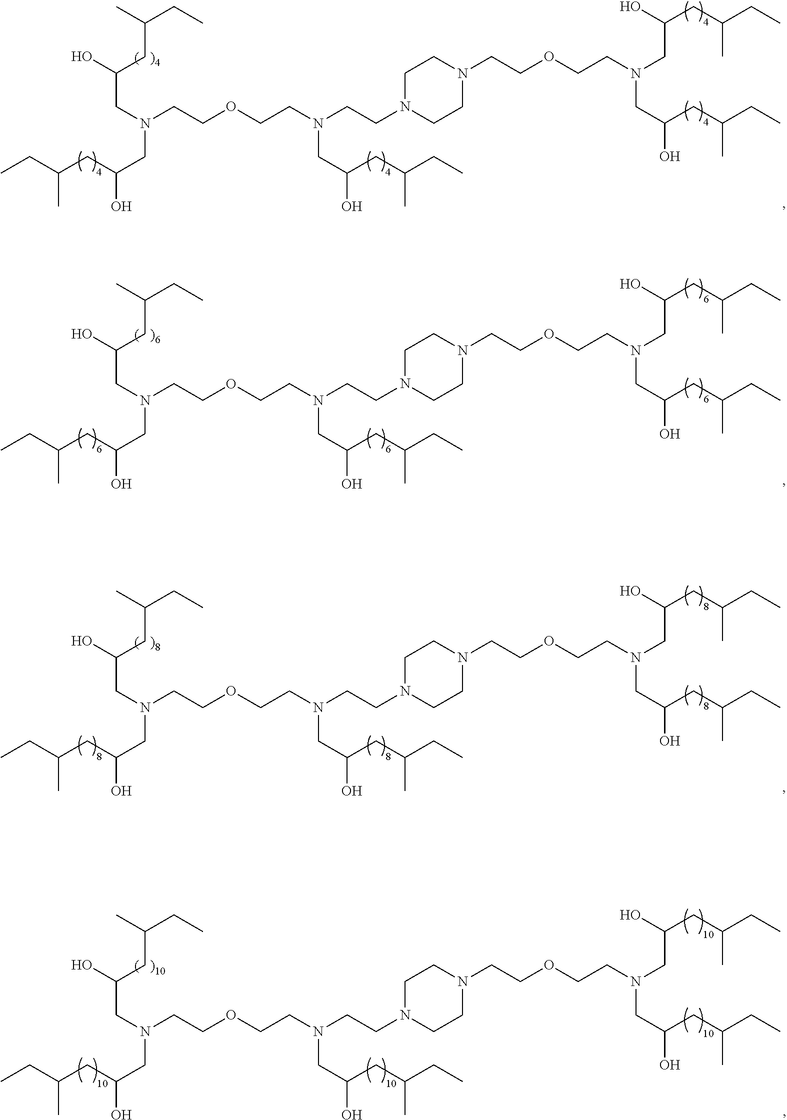

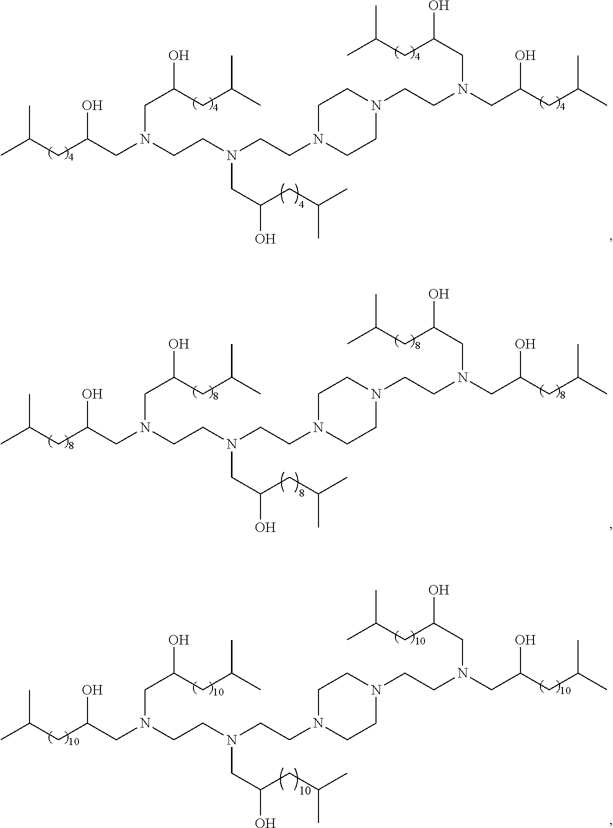

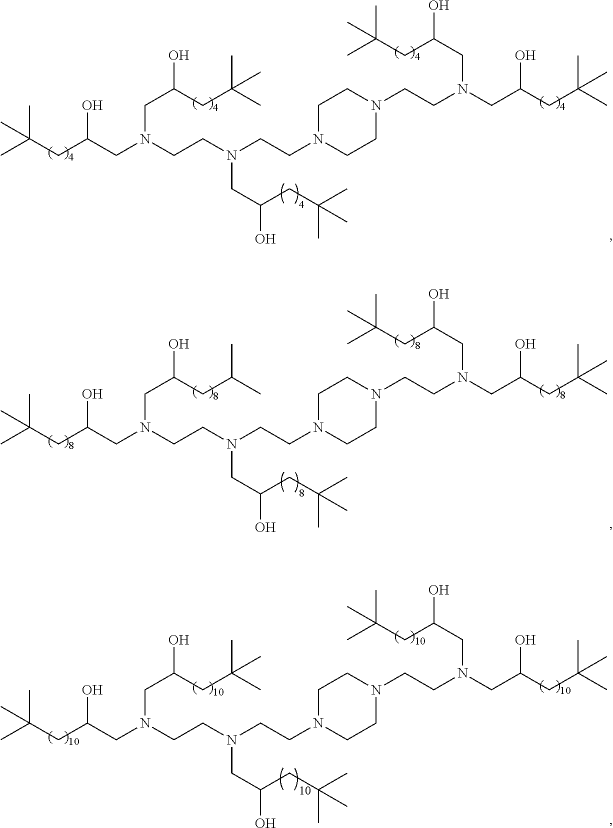

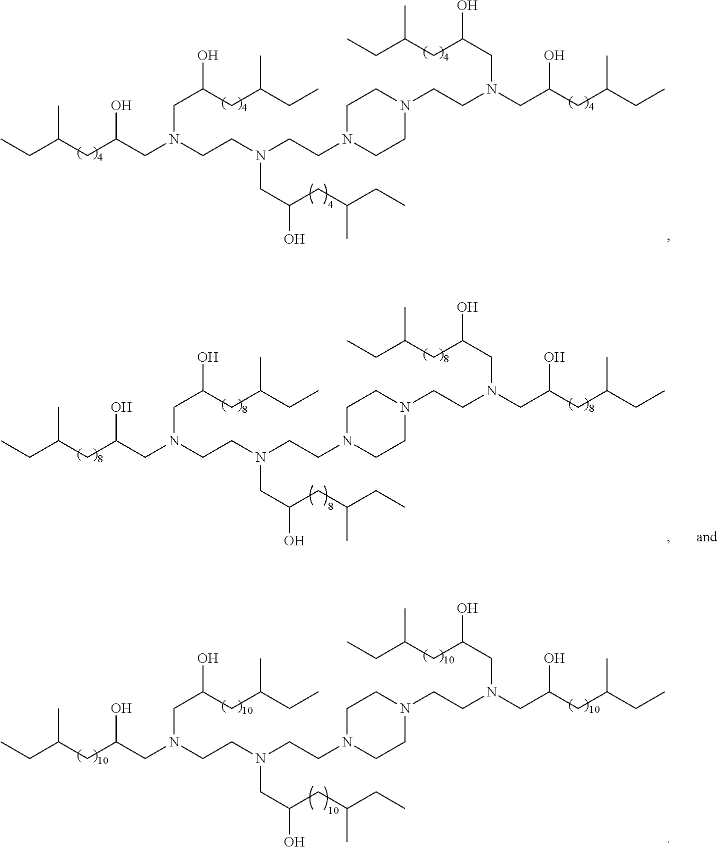

Embodiment 15 provides a lipid nanoparticle (LNP) composition comprising:
(a) at least one ionizable lipid compound or a salt, solvate, stereoisomer, or isotopologue thereof having the structure of Formula (I):

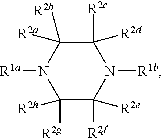

wherein:
R^{1a} and R^{1b} are each independently

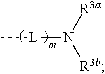

R^{2a}, R^{2b}, R^{2c}, R^{2d}, R^{2e}, R^{2f}, R^{2g}, and R^{2h} are each independently selected from the group consisting of H, optionally substituted C₁-C₁₂ alkyl, optionally substituted C₂-C₁₂ heteroalkyl, optionally substituted C₃-C₈ cycloalkyl, optionally substituted C₂-C₈ heterocycloalkyl, optionally substituted C₂-C₁₂ alkenyl, optionally substituted C₂-C₁₂ alkynyl, optionally substituted C₆-C₁₀ aryl, and optionally substituted C₂-C₁₀ heteroaryl; each occurrence of R^{3a}, R^{3b}, and R^{3C} is independently selected from the group consisting of

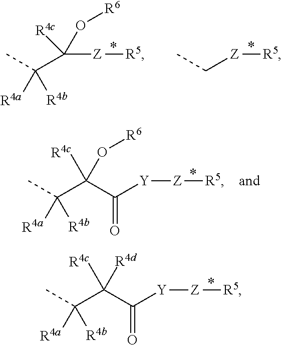

wherein no more than one of R^{3a}, R^{3b}, and R^{3c} is H;
each occurrence of R^{4a}, R^{4b}, R^{4c}, and R^{4d}, if present, is independently selected from the group consisting of optionally substituted C₁-C₁₂ alkyl, halogen, CN, and NO₂;
each occurrence of R⁵ is independently selected from the group consisting of optionally substituted C₁-C₃ alkyl, optionally substituted C₃-C₁₂ cycloalkyl, optionally substituted C₆-C₁₀ aryl, and optionally substituted C₂-C₁₀ heteroaryl;
each occurrence of R⁶ is independently selected from the group consisting of H, optionally substituted C₁-C₆ alkyl, optionally substituted C₃-C₁₂ cycloalkyl, C₂-C₁₂ heterocycloalkyl, optionally substituted C₆-C₁₂ aralkyl, optionally substituted C₆-C₁₂ aryl, optionally substituted C₂-C₁₂ heteroaryl, C(═O)R^{a}, C(═O)OR^{a}, and C(═O)N(R^{a})(R^{b}); each occurrence of L is independently selected from the group consisting of a bond, -(optionally substituted C₁-C₁₂ alkylenyl)-X—, -(optionally substituted C₂-C₁₂ alkenylenyl)-X—, -(optionally substituted C₁-C₁₂ alkynylenyl)-X—, -(optionally substituted C₁-C₁₂ heteroalkylenyl)-X—, optionally substituted C₃-C₅ cycloalkylenyl, and optionally substituted C₂-C₅ heterocyloalkylenyl;
each occurrence of X, if present, is independently selected from the group consisting of a bond, —N(R^{3c})—, and —O—;
each occurrence of Y, if present, is independently selected from the group consisting of a bond, —N(R^{a})—, and —O—;
each occurrence of Z is C₁-C₂₄ alkylenyl,
   wherein the C₁-C₂₄ alkylenyl in each occurrence of Z is independently substituted with at least one substituent selected from the group consisting of C₁-C₁₂ alkyl and C₁-C₁₂ haloalkyl, and
   wherein the C₁-C₂₄ alkylenyl in each occurrence of Z is independently optionally further substituted;
each occurrence of R^{a} and R^{b} is independently selected from the group consisting of H, optionally substituted C₁-C₆ alkyl, optionally substituted C₃-C₈ cycloalkyl, optionally substituted C₁-C₆ haloalkyl, optionally substituted C₆-C₁₂ aralkyl, optionally substituted phenyl, optionally substituted C₂-C₈ heteroaryl, C(═O)R^{c}, C(═O)OR^{c}, and C(═O)N(R^{c})(R^{d});
each occurrence of R^{c} and R^{d} is independently selected from the group consisting of H, optionally substituted C₁-C₆ alkyl, optionally substituted C₃-C₈ cycloalkyl, optionally substituted C₁-C₆ haloalkyl, optionally substituted C₆-C₁₂ aralkyl, optionally substituted phenyl, and optionally substituted C₂-C₈ heteroaryl; and each occurrence of m is independently an integer selected from the group consisting of 1, 2, 3, and 4;
(b) at least one neutral lipid;
(c) cholesterol; and
(d) at least one conjugated lipid.

Embodiment 16 provides the LNP of Embodiment 15, wherein the LNP further comprises:
(e) at least one nucleic acid and/or therapeutic agent cargo, wherein the cargo is at least partially encapsulated therein.

Embodiment 17 provides the LNP of Embodiment 15 or 16, wherein at least one of the following applies:
(a) at least one selected from the group consisting of R^{2a}, R^{2b}, R^{2c}, R^{2d}, R^{2e}, R^{2f}, R^{2g}, and R^{2h} is H;
(b) at least two selected from the group consisting of R^{2a}, R^{2b}, R^{2c}, R^{2d}, R^{2e}, R², R^{2g}, and R^{2h} are H;
(c) at least three selected from the group consisting of R^{2a}, R^{2b}, R^{2c}, R^{2d}, R^{2e}, R^{2f}, R^{2g}, and R^{2h} are H;
(d) at least four selected from the group consisting of R^{2a}, R^{2b}, R^{2c}, R^{2d}, R^{2e}, R^{2f}, R^{2g}, and R^{2h} are H;
(e) at least five selected from the group consisting of R^{2a}, R^{2b}, R^{2c}, R^{2d}, R^{2e}, R², R^{2g}, and R^{2h} are H;
(f) at least six selected from the group consisting of R^{2a}, R^{2b}, R^{2c}, R^{2d}, R^{2e}, R², R^{2g}, and R^{2h} are H;
(g) at least seven selected from the group consisting of R^{2a}, R^{2b}, R^{2c}, R^{2d}, R^{2e}, R^{2f}, R^{2g}, and R^{2h} are H; and
(h) each of R^{2a}, R^{2b}, R^{2c}, R^{2d}, R^{2e}, R^{2f}, R^{2g}, and R^{2h} are H.

Embodiment 18 provides the LNP of any one of Embodiments 15-17, wherein each occurrence of L is independently selected from the group consisting of L is independently selected from the group consisting of —(CH₂)₁₋₁₀—, —(CH₂)₂₋₁₀NR^{3c}—, —(CH₂)₂₋₁₀₀—, —(CH₂)₁₋₃—CH(OR^{a})—(CH₂)₁₋₃—, piperazinylenyl, and cyclohexylenyl.

Embodiment 19 provides the LNP of any one of Embodiments 15-18, wherein each occurrence of L is independently selected from the group consisting of —CH₂—, —(CH₂)₂—, —(CH₂)₃—, —(CH₂)₁₀—, —(CH₂)₂O—, —(CH₂)₃O—, —CH₂CH(OR^{a})CH₂—, —(CH₂)₂NR^{3c}—,

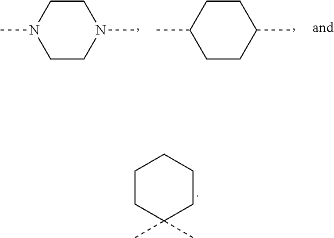

Embodiment 20 provides the LNP of any one of Embodiments 15-19, wherein the compound of Formula (I) is selected from the group consisting of:

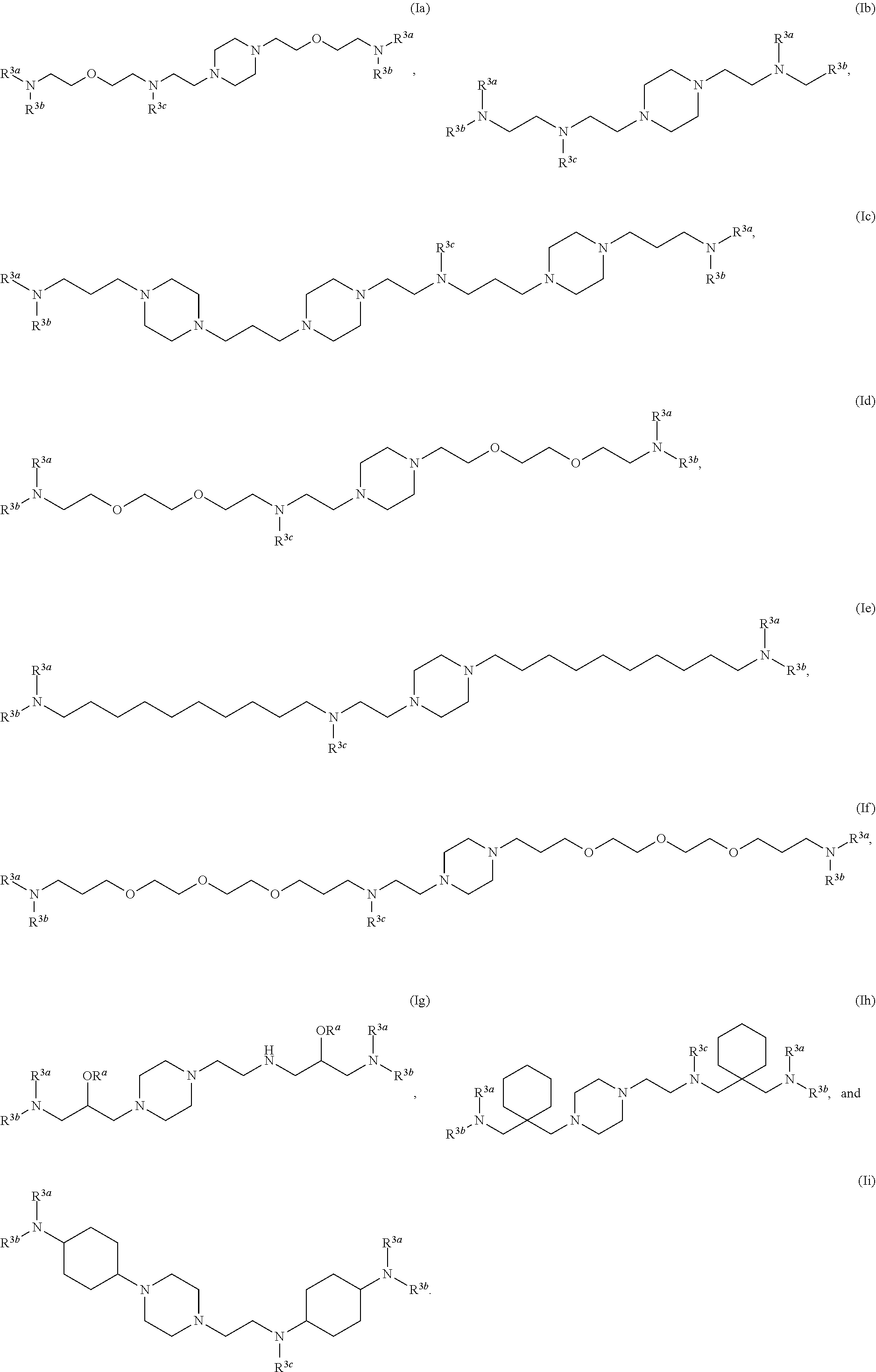

Embodiment 21 provides the LNP of any one of Embodiments 15-20, wherein each occurrence of R^{4a}, R^{4b}, R^{4c}, and R^{4d} is independently H.

Embodiment 22 provides the LNP of any one of Embodiments 15-21, wherein each occurrence of R⁵ is independently methyl.

Embodiment 23 provides the LNP of any one of Embodiments 15-22, wherein each occurrence of R⁶ is independently H.

Embodiment 24 provides the LNP of any one of Embodiments 15-23, wherein each occurrence of Z is independently:

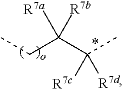

wherein:
each occurrence of R^{7a}, R^{7b}, R^{7c}, and R^{7d} is independently selected from the group consisting of H, C₁-C₆ alkyl, and C₁-C₆ haloalkyl,
   wherein at least one of R^{7a}, R^{7b}, R^{7c}, and R^{7d} is not H; and
each occurrence of o is an integer selected from the group consisting of 1, 2, 3, 4, 5, 6, 7, 8, 9, 10, 11, and 12.

Embodiment 25 provides the LNP of Embodiment 24, wherein each occurrence of R^{7a}, R^{7b}, R^{7c}, and R^{7d} is selected from the group consisting of H and methyl.

Embodiment 26 provides the LNP of any one of Embodiments 15-25, wherein each occurrence of Z is independently selected from the group consisting of —(CH₂)₄₋₁₀—CH(CH₃)—*, —(CH₂)₄₋₁₀—C(CH₃)₂—*, and —(CH₂)₄₋₁₀—CH(CH₃)—CH₂—*.

Embodiment 27 provides the LNP of any one of Embodiments 15-26, wherein each occurrence of R^{3a}, R^{3b}, and R^{3c} is independently selected from the group consisting of

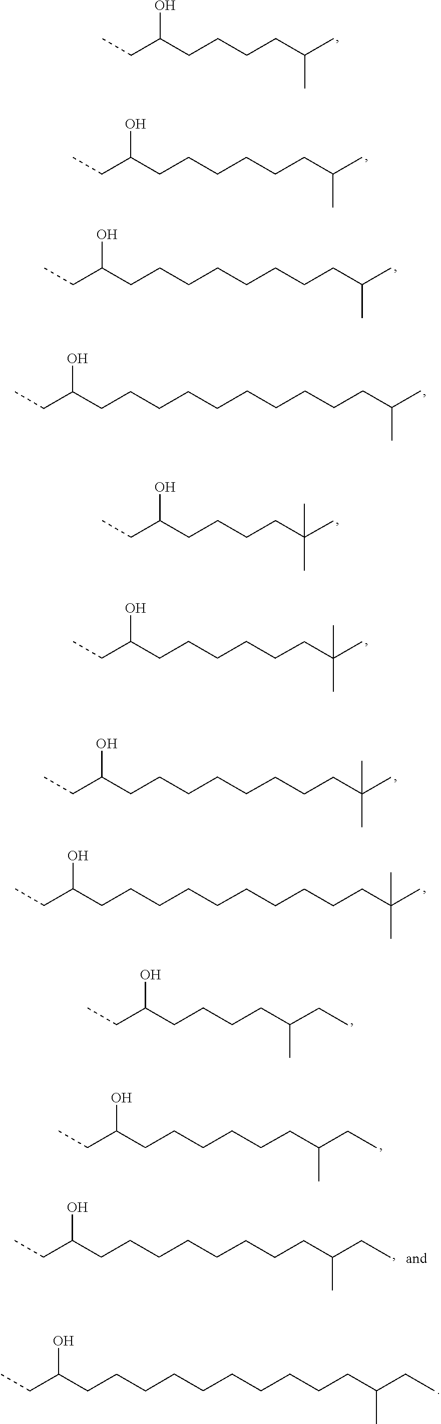

Embodiment 28 provides the LNP of any one of Embodiments 15-27, wherein each occurrence of optionally substituted alkyl, optionally substituted heteroalkyl, optionally substituted cycloalkyl, optionally substituted heterocycloalkyl, optionally substituted alkenyl, optionally substituted alkynyl, optionally substituted aryl, optionally substituted heteroaryl, optionally substituted aralkyl, optionally substituted alkylenyl, optionally substituted alkenylenyl, optionally substituted alkynylenyl, optionally substituted heteroalkylenyl, optionally substituted cycloalkylenyl, and optionally substituted heterocycloalkylenyl, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OH, N(R′)(R″), C(═O)R′, C(═O)OR′, OC(═O)OR′, C(═O)N(R′)(R″), S(═O)₂N(R′)(R″), N(R′)C(═O)R″, N(R′)S(═O)₂R″, C₂-C₈ heteroaryl, and phenyl optionally substituted with at least one halogen, wherein each occurrence of R′ and R″ is independently selected from the group consisting of H, C₁-C₆ alkyl, C₃-C₈ cycloalkyl, C₁-C₆ haloalkyl, benzyl, and phenyl.

Embodiment 29 provides the LNP of any one of Embodiments 15-28, which is selected from the group consisting of:

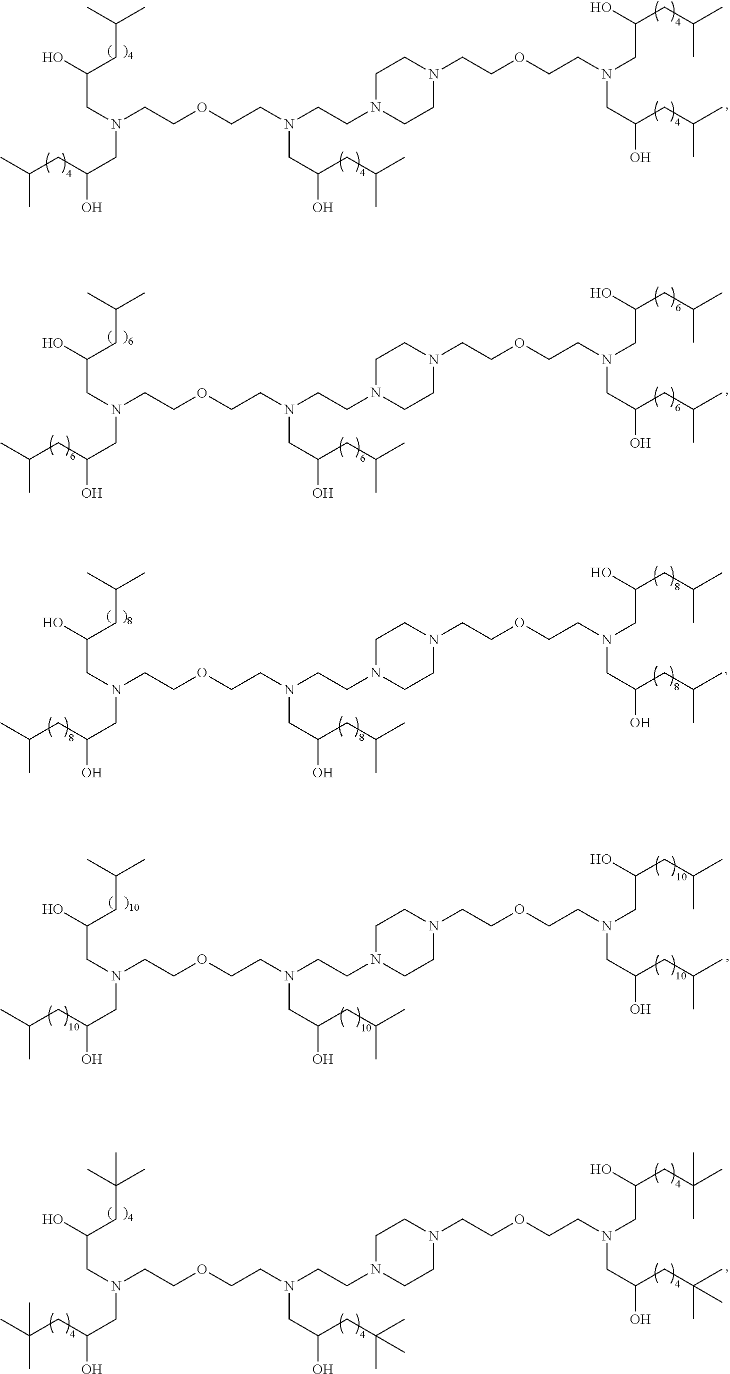

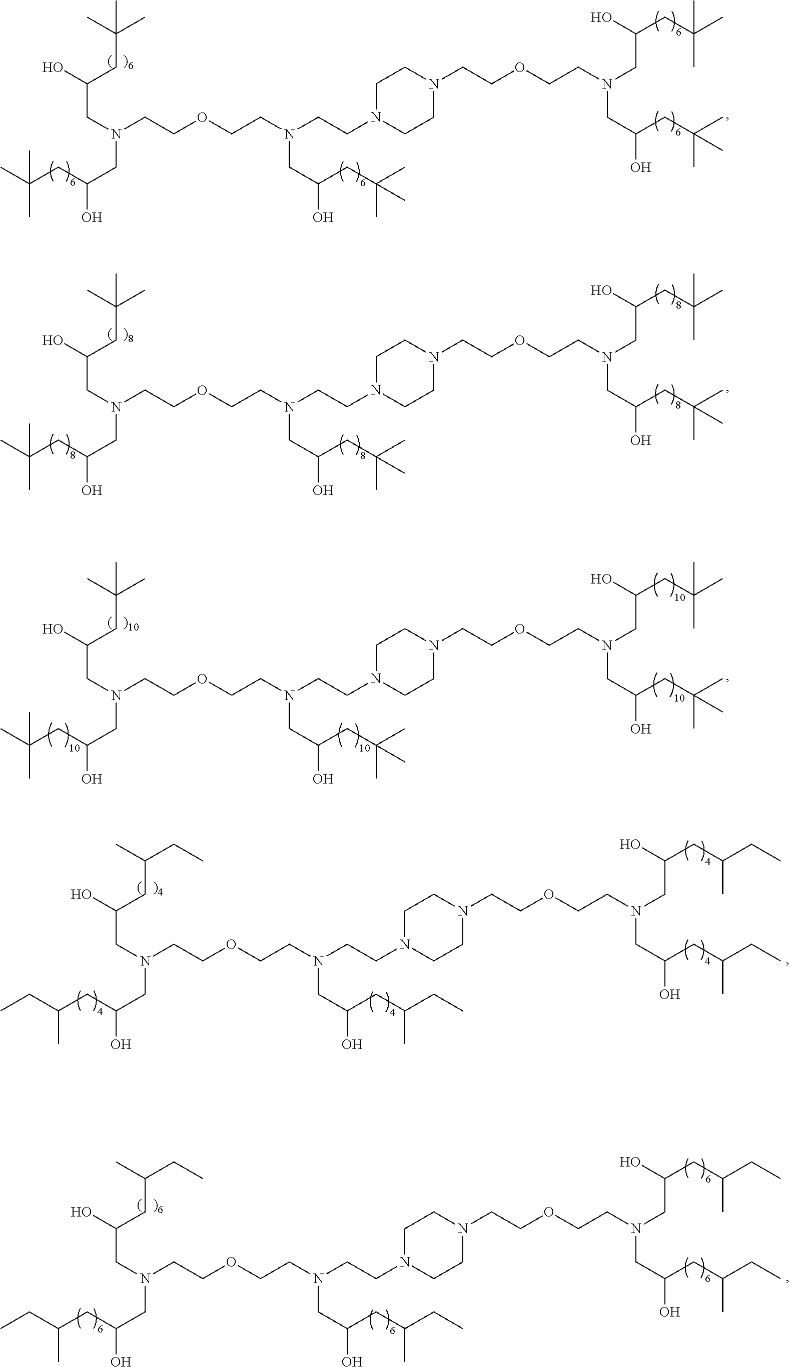

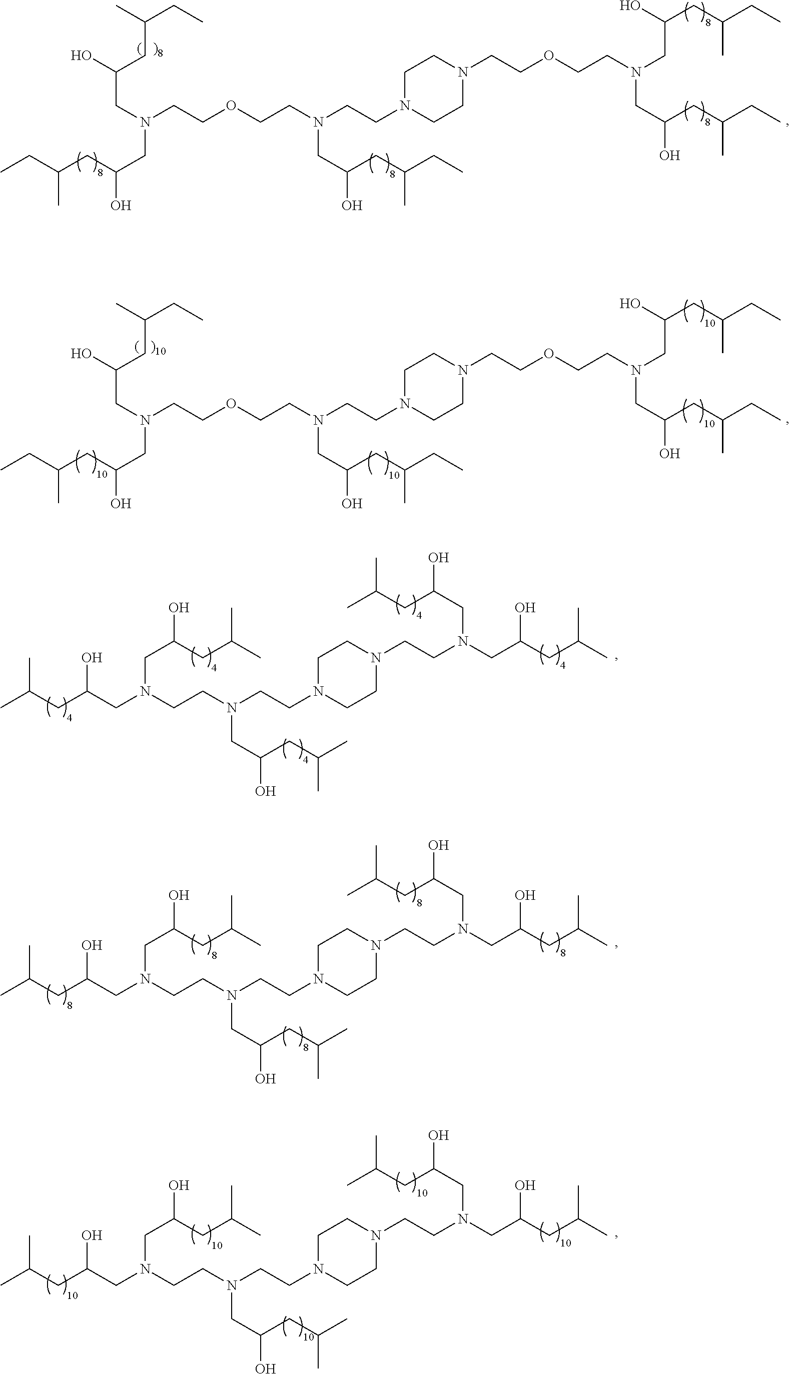

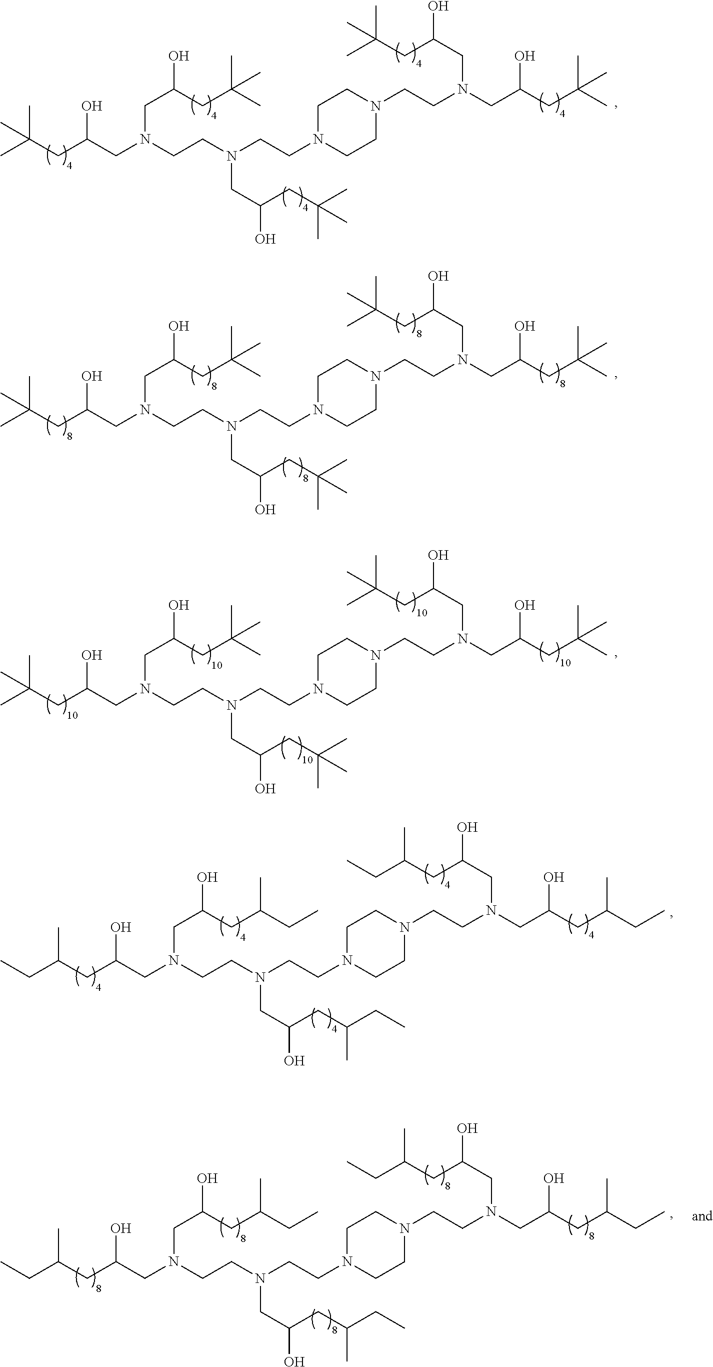

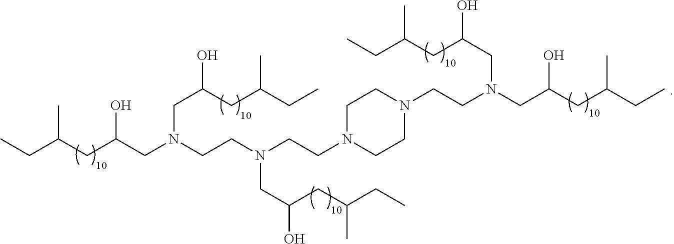

Embodiment 30 provides the LNP of any one of Embodiments 15-29, wherein the at least one ionizable lipid of Formula (I) comprises about 10 mol % to about 90 mol % of the LNP.

Embodiment 31 provides the LNP of any one of Embodiments 15-30, wherein the at least one ionizable lipid of Formula (I) comprises about 35 mol % or about 40 mol % of the LNP.

Embodiment 32 provides the LNP of any one of Embodiments 15-31, wherein the at least one neutral lipid comprises about 1 mol % to about 40 mol % of the LNP.

Embodiment 33 provides the LNP of any one of Embodiments 15-32, wherein the at least one neutral lipid comprises about 16 mol % or about 30 mol % of the LNP.

Embodiment 34 provides the LNP of any one of Embodiments 15-33, wherein the at least one neutral lipid comprises at least one selected from the group consisting of dioleoylphosphatidylethanolamine (DOPE), distearoylphosphatidylcholine (DSPC), and dioleoylphosphatidylcholine (DOPC).

Embodiment 35 provides the LNP of any one of Embodiments 15-34, wherein the at least one neutral lipid comprises dioleoylphosphatidylethanolamine (DOPE).

Embodiment 36 provides the LNP of any one of Embodiments 15-35, wherein the cholesterol comprises about 20 mol % to about 75 mol % of the LNP.

Embodiment 37 provides the LNP of any one of Embodiments 15-36, wherein the cholesterol comprises about 25 mol % or about 46.5 mol % of the LNP.

Embodiment 38 provides the LNP of any one of Embodiments 15-37, wherein the at least one conjugated lipid comprises about 0.1 mol % to about 15 mol % of the LNP.

Embodiment 39 provides the LNP of any one of Embodiments 15-38, wherein the at least one conjugated lipid comprises about 2.5 mol % of the LNP.

Embodiment 40 provides the LNP of any one of Embodiments 15-39, wherein the at least one conjugated lipid comprises 1,2-dimyristoyl-rac-glycero-3-methoxypolyethylene glycol-2000 (DMG-PEG₂₀₀₀).

Embodiment 41 provides the LNP of any one of Embodiments 15-40, wherein the LNP has a molar ratio of (a):(b):(c):(d) of about 35:16:46.5:2.5 or about 40:30:25:2.5 Embodiment 42 provides the LNP of any one of Embodiments 16-41, wherein the nucleic acid molecule is a therapeutic agent.

Embodiment 43 provides the LNP of any one of Embodiments 16-42, wherein the nucleic acid molecule is at least one selected from the group consisting of RNA and DNA.

Embodiment 44 provides the LNP of any one of Embodiments 16-43, wherein the nucleic acid molecule is at least one selected from the group consisting of mRNA, cDNA, miRNA, siRNA, and modified RNA.

Embodiment 45 provides the LNP of any one of Embodiments 42-44, wherein the nucleic acid is mRNA.

Embodiment 46 provides the LNP of Embodiment 45, wherein the LNP has a mass ratio of (a):mRNA of about 20:1 to about 5:1 (w/w), optionally wherein the LNP has a mass ratio of (a):mRNA of about 10:1.

Embodiment 47 provides the LNP of Embodiment 45 or 46, wherein the mRNA encodes a chimeric antigen receptor (CAR).

Embodiment 48 provides the LNP of Embodiment 47, wherein the CAR is specific for binding to a surface antigen of a pathogenic cell or a tumor cell.

Embodiment 49 provides the LNP of Embodiment 48, wherein the surface antigen is selected from the group consisting of CD1, CD2, CD3, CD5, CD7, CD8, CD16, CD19, CD20, CD22, CD25, CD26, CD27, CD28, CD30, CD33, CD38, CD39, CD40L, CD44, CD45, CD62L, CD69, CD73, CD80, CD83, CD86, CD95, CD103, CD119, CD123, CD126, CD150, CD153, CD154, CD161, CD183, CD223, CD254, CD275, CD45RA, CXCR3, CXCR5, FasL, IL18R1, CTLA-4, OX40, GITR, LAG3, ICOS, PD-1, leu-12, TCR, TLR1, TLR2, TLR3, TLR4, TLR6, NKG2D, CCR, CCR1, CCR2, CCR4, CCR6, CCR7, k light chain, ROR1, ErbB2, ErbB3, ErbB4, EGFR vIII, carcinoembryonic antigen, EGP2, EGP40, mesothelin, TAG72, PSMA, NKG2D ligands, B7-H6, IL13R-α2, MUC1, VEGF-A, Tem8, FAP, EphA2, HER2, MUC16, CA9, GD2, GD3, HMW-MAA, CD171, Lewis Y, G250/CALX, HLA-AI MAGE A1, HAL-A2 NY-ESO-1, PSC1, folate receptor-α, 8H9, NCAM, VEGF, 5T4, Fetal AchR, NKG2D ligands, TEM1, and TEM8.

Embodiment 50 provides the LNP of Embodiment 45 or 46, wherein the mRNA encodes an enzyme.

Embodiment 51 provides the LNP of any one of Embodiments 45-46 and 49, wherein the mRNA encodes a clustered regularly interspaced short palindrome repeats (CRISPR) associated protein, optionally wherein the CRISPR associated protein is Cas9.

Embodiment 52 provides a pharmaceutical composition comprising the lipid nanoparticle (LNP) of any one of Embodiments 15-51 and at least one pharmaceutically acceptable carrier.

Embodiment 53 provides the pharmaceutical composition of Embodiment 52, wherein the composition further comprises at least one adjuvant.

Embodiment 54 provides a method of treating, preventing, and/or ameliorating a disease in a subject, the method comprising administering to the subject at least one lipid nanoparticle (LNP) of any one of Embodiments 16-51 and/or at least one pharmaceutical composition of Embodiment 52 or 53.

Embodiment 55 provides the method of Embodiment 54, wherein the disease is selected from the group consisting of cancer, an autoimmune disorder, cardiovascular disease, and neurological disease.

Embodiment 56 provides the method of Embodiment 55, wherein the cancer is at least one selected from the group consisting of oral cancer, pancreatic cancer, colorectal cancer, bladder cancer, breast cancer, prostate cancer, renal cancer, hepatocellular cancer, lung cancer, ovarian cancer, cervical cancer, gastric cancer, esophageal cancer, head and neck cancer, melanoma, neuroendocrine cancer, CNS cancer, brain cancer, bone cancer, soft tissue sarcoma, non-small cell lung cancer, small-cell lung cancer, or colon cancer.

Embodiment 57 provides the method of Embodiment 54 or 56, wherein the subject is further administered at least one additional agent or therapy useful for treating, preventing, and/or ameliorating cancer in the subject.

Embodiment 58 provides the method of any one of Embodiments 54-57, wherein the subject is a mammal.

Embodiment 59 provides the method of Embodiment 58, wherein the mammal is a human.

Embodiment 60 provides a method of delivering a nucleic acid or therapeutic agent to the liver of a subject, the method comprising administering to the subject at least one lipid nanoparticle (LNP) of any one of Embodiments 16-51 and/or at least one pharmaceutical composition of Embodiment 52 or 53.

Embodiment 61 provides a method of preparing a modified immune cell or precursor thereof, the method comprising contacting an immune cell or precursor thereof with the lipid nanoparticle (LNP) of any one of Embodiments 16-51 and/or at least one pharmaceutical composition of Embodiment 52 or 53.

Embodiment 62 provides the method of Embodiment 61, wherein the modified immune cell or precursor thereof is ap T cell, a γδ T cell, a CD8+ T cell, a CD4+ helper T cell, a CD4+ regulatory T cell, an NK T cell, an NK cell, and any combination thereof.

Embodiment 63 provides the method of Embodiment 62, wherein the modified immune cell or precursor thereof is a T cell, optionally wherein the T cell is a CD4+ T cell.

Embodiment 64 provides the method of Embodiment 62, wherein the modified immune cell or precursor thereof is a NK cell.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

1. An ionizable lipid compound of Formula (I), or a salt, solvate, stereoisomer, or isotopologue thereof: 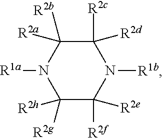
wherein:
R^{1a} and R^{1b} are each independently 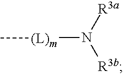
R^{2a}, R^{2b}, R^{2c}, R^{2d}, R^{2e}, R^{2f}, R^{2g}, and R^{2h} are each independently selected from the group consisting of H, optionally substituted C₁-C₁₂ alkyl, optionally substituted C₂-C₁₂ heteroalkyl, optionally substituted C₃-C₈ cycloalkyl, optionally substituted C₂-C₈ heterocycloalkyl, optionally substituted C₂-C₁₂ alkenyl, optionally substituted C₂-C₁₂ alkynyl, optionally substituted C₆-C₁₀ aryl, and optionally substituted C₂-C₁₀ heteroaryl;
each occurrence of R^{3a}, R^{3b}, and R^{3c} is independently selected from the group consisting of H, 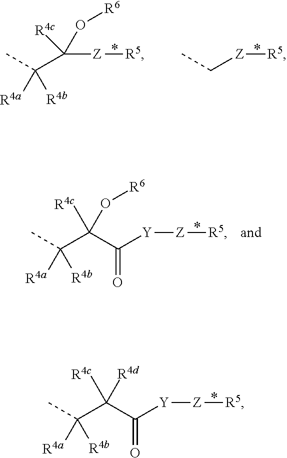
wherein no more than one of R^{3a}, R^{3b}, and R^{3c} is H;
each occurrence of R^{4a}, R^{4b}, R^{4c}, and R^{4d}, if present, is independently selected from the group consisting of optionally substituted C₁-C₁₂ alkyl, halogen, CN, and NO₂;
each occurrence of R⁵ is independently selected from the group consisting of optionally substituted C₁-C₃ alkyl, optionally substituted C₃-C₁₂ cycloalkyl, optionally substituted C₆-C₁₀ aryl, and optionally substituted C₂-C₁₀ heteroaryl;
each occurrence of R⁶ is independently selected from the group consisting of H, optionally substituted C₁-C₆ alkyl, optionally substituted C₃-C₁₂ cycloalkyl, C₂-C₁₂ heterocycloalkyl, optionally substituted C₆-C₁₂ aralkyl, optionally substituted C₆-C₁₂ aryl, optionally substituted C₂-C₁₂ heteroaryl, C(═O)R^{a}, C(═O)OR^{a}, and C(═O)N(R^{a})(R^{b});
each occurrence of L is independently selected from the group consisting of a bond, -(optionally substituted C₁-C₁₂ alkylenyl)-X—, -(optionally substituted C₂-C₁₂ alkenylenyl)-X—, -(optionally substituted C₁-C₁₂ alkynylenyl)-X—, -(optionally substituted C₁-C₁₂ heteroalkylenyl)-X—, optionally substituted C₃-C₈ cycloalkylenyl, and optionally substituted C₂-C₈ heterocyloalkylenyl;
each occurrence of X, if present, is independently selected from the group consisting of a bond, —N(R^{3c})—, and —O—;
each occurrence of Y, if present, is independently selected from the group consisting of a bond, —N(R^{a})—, and —O—;
each occurrence of Z is C₁-C₂₄ alkylenyl,
wherein the C₁-C₂₄ alkylenyl in each occurrence of Z is independently substituted with at least one substituent selected from the group consisting of C₁-C₁₂ alkyl and C₁-C₁₂ haloalkyl, and
wherein the C₁-C₂₄ alkylenyl in each occurrence of Z is independently optionally further substituted;
each occurrence of R^{a} and R^{b} is independently selected from the group consisting of H, optionally substituted C₁-C₆ alkyl, optionally substituted C₃-C₈ cycloalkyl, optionally substituted C₁-C₆ haloalkyl, optionally substituted C₆-C₁₂ aralkyl, optionally substituted phenyl, optionally substituted C₂-C₈ heteroaryl, C(═O)R^{c}, C(═O)OR^{c}, and C(═O)N(R^{c})(R^{d});
each occurrence of R^{c} and R^{d} is independently selected from the group consisting of H, optionally substituted C₁-C₆ alkyl, optionally substituted C₃-C₈ cycloalkyl, optionally substituted C₁-C₆ haloalkyl, optionally substituted C₆-C₁₂ aralkyl, optionally substituted phenyl, and optionally substituted C₂-C₈ heteroaryl; and
each occurrence of m is independently 1, 2, 3, or and 4.

2. (canceled)

3. The compound of claim 1, wherein each occurrence of L is independently selected from the group consisting of L is independently selected from the group consisting of —(CH₂)₁₋₁₀—, —(CH₂)₂₋₁₀NR^{3c}—, —(CH₂)₂₋₁₀O—, —(CH₂)₁₋₃—CH(OR^{a})—(CH₂)₁₋₃—, piperazinylenyl, and cyclohexylenyl,
optionally wherein L is independently selected from the group consisting of —CH₂—, —(CH₂)₂—, —(CH₂)₃—, —(CH₂)₁₀—, —(CH₂)₂O—, —(CH₂)₃O—, —CH₂CH(OR^{a})CH₂—, —(CH₂)₂NR^{3c}—,

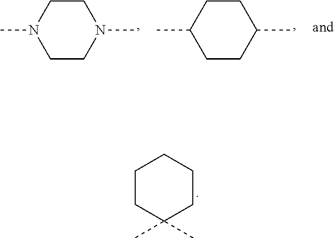

4. (canceled)

5. The compound of claim 1, wherein the compound of Formula (I) is selected from the group consisting of:

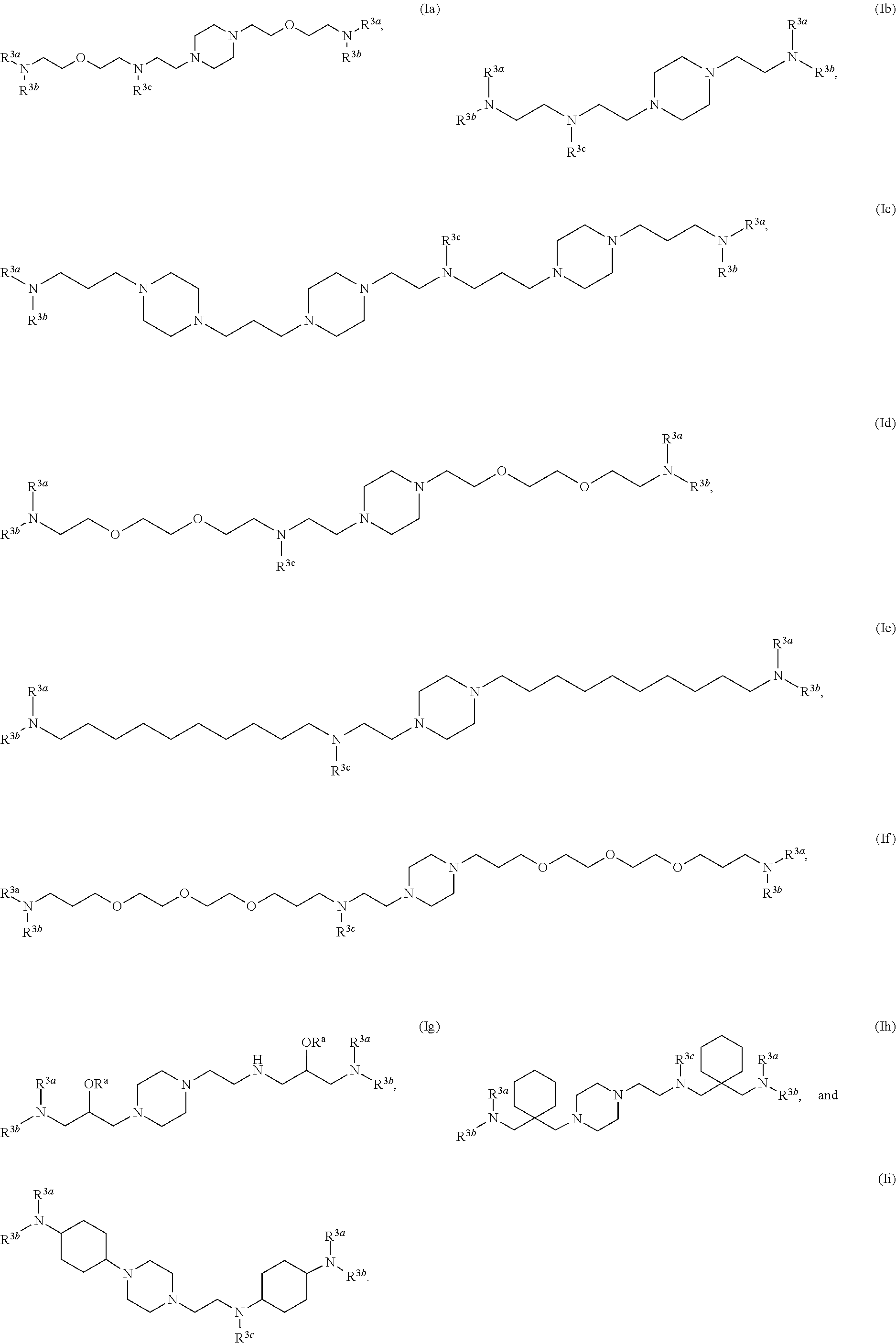

6. The compound of claim 1, wherein at least one of the following applies:
(a) each occurrence of R^{4a}, R^{4b}, R^{4c}, and R^{4d} is independently H;
(b) each occurrence of R⁵ is independently methyl;
(c) each occurrence of R⁶ is independently H;
(d) each occurrence of Z is independently selected from the group consisting of —(CH₂)₄₋₁₀—CH(CH₃)—*, —(CH₂)₄₋₁₀—C(CH₃)₂—*, and —(CH₂)₄₋₁₀—CH(CH₃)—CH₂—*; and
(e) each occurrence of R^{3a}, R^{3b}, and R^{3c} is independently selected from the group consisting of

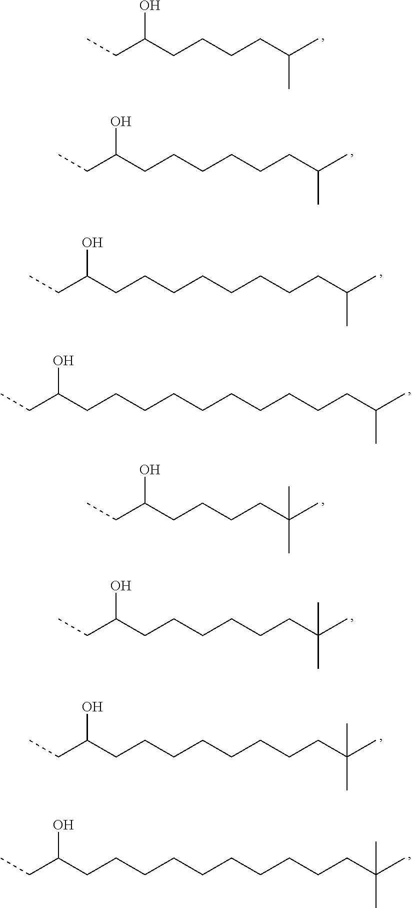

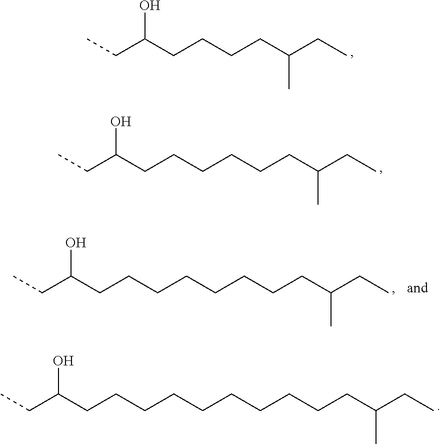

7-8. (canceled)

9. The compound of claim 1, wherein each occurrence of Z is independently: 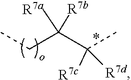
wherein:
each occurrence of R^{7a}, R^{7b}, R^{7c}, and R^{7d} is independently selected from the group consisting of H, C₁-C₆ alkyl, and C₁-C₆ haloalkyl,
wherein at least one of R^{7a}, R^{7b}, R^{7c}, and R^{7d} is not H; and
each occurrence of o is an integer selected from the group consisting of 1, 2, 3, 4, 5, 6, 7, 8, 9, 10, 11, and 12.

10-12. (canceled)

13. The compound of claim 1, wherein each occurrence of optionally substituted alkyl, optionally substituted heteroalkyl, optionally substituted cycloalkyl, optionally substituted heterocycloalkyl, optionally substituted alkenyl, optionally substituted alkynyl, optionally substituted aryl, optionally substituted heteroaryl, optionally substituted aralkyl, optionally substituted alkylenyl, optionally substituted alkenylenyl, optionally substituted alkynylenyl, optionally substituted heteroalkylenyl, optionally substituted cycloalkylenyl, and optionally substituted heterocycloalkylenyl, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OH, N(R′)(R″), C(═O)R′, C(═O)OR′, OC(═O)OR′, C(═O)N(R′)(R″), S(═O)₂N(R′)(R″), N(R′)C(═O)R″, N(R′)S(═O)₂R″, C₂-C₈ heteroaryl, and phenyl optionally substituted with at least one halogen, wherein each occurrence of R′ and R″ is independently selected from the group consisting of H, C₁-C₆ alkyl, C₃-C₈ cycloalkyl, C₁-C₆ haloalkyl, benzyl, and phenyl.

14. The compound of claim 1, which is selected from the group consisting of:

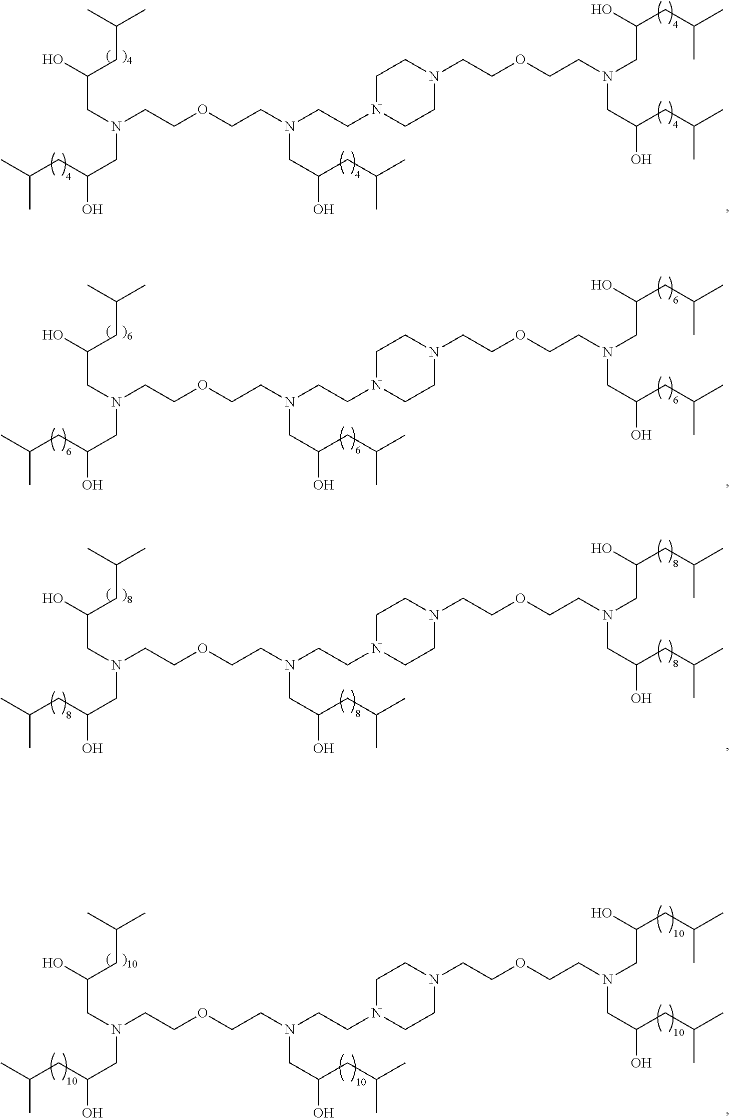

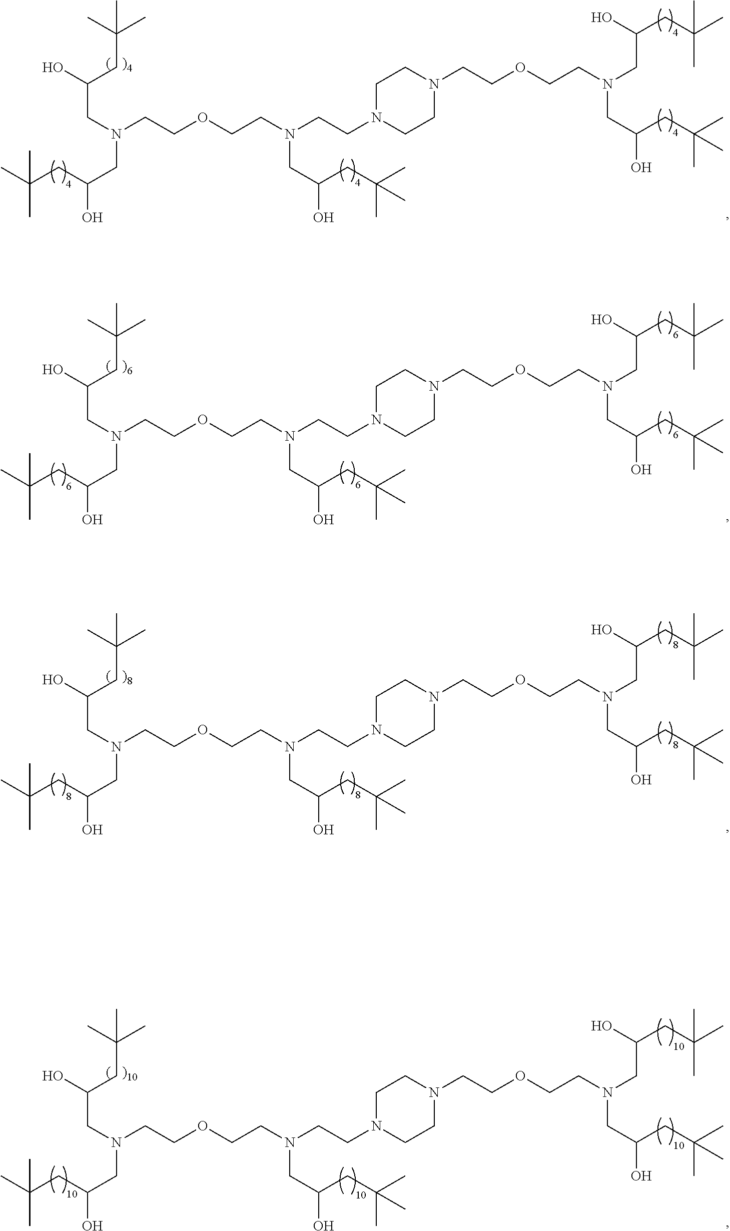

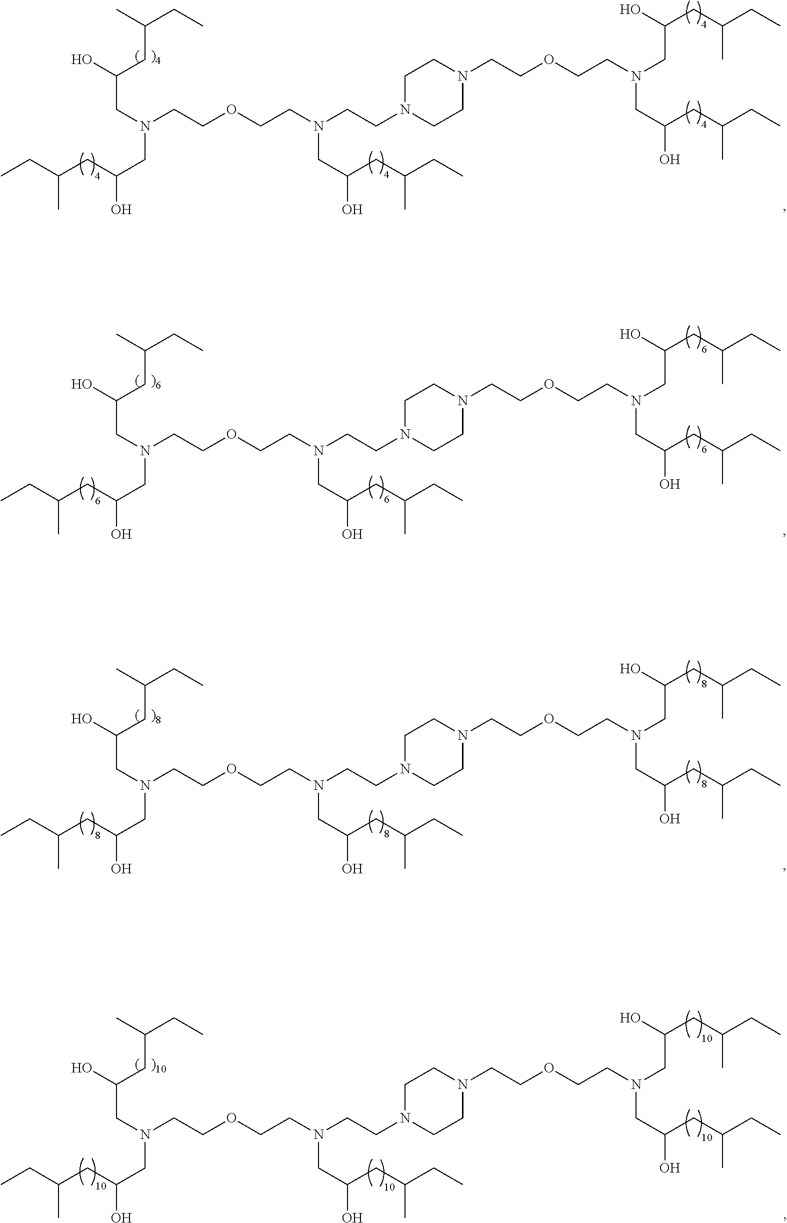

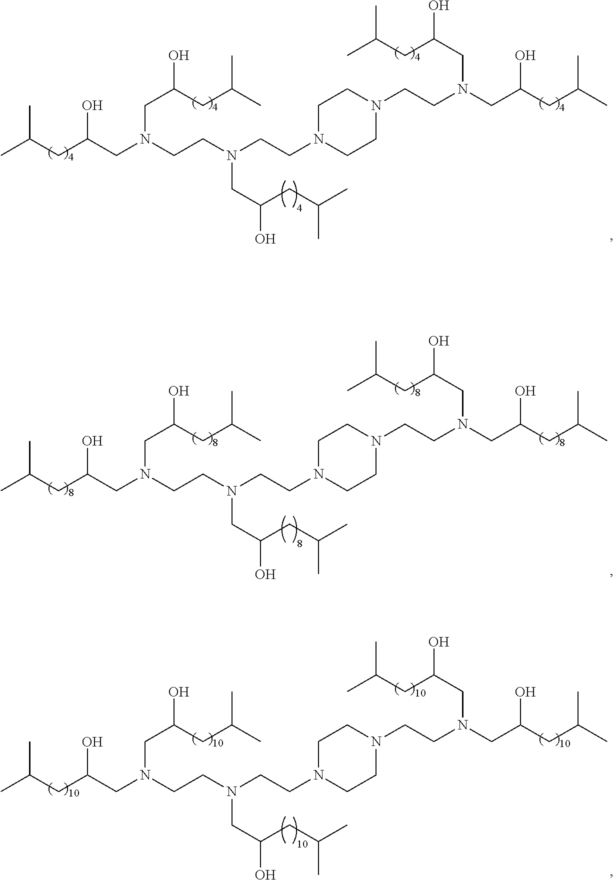

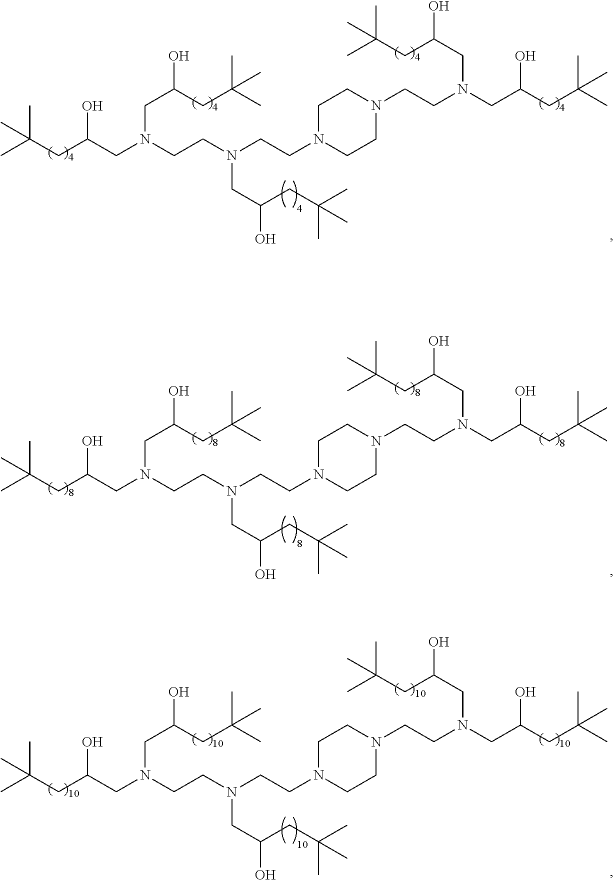

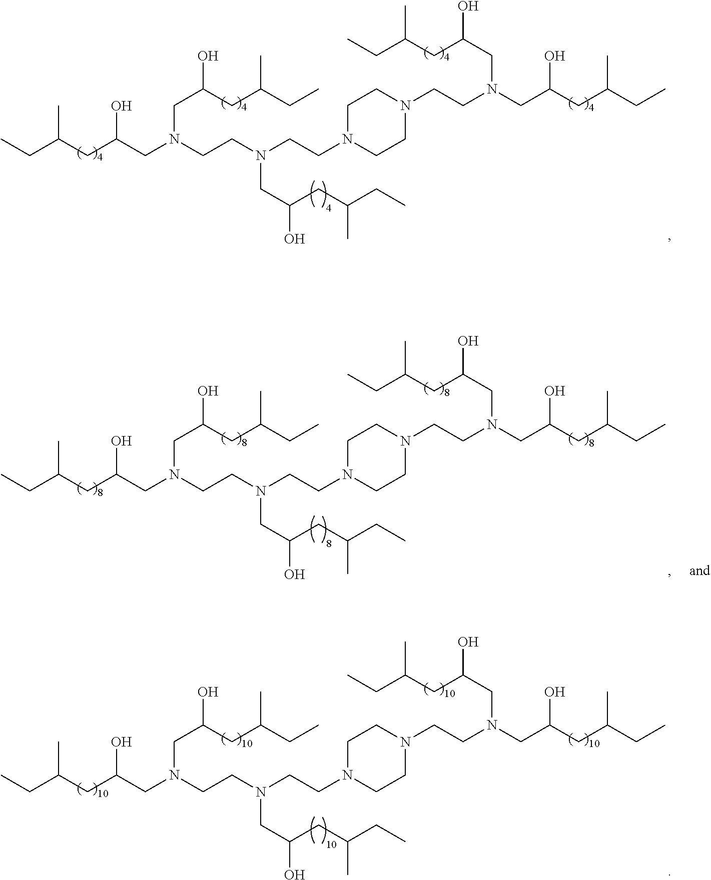

15. A lipid nanoparticle (LNP) composition comprising:
(a) at least one ionizable lipid compound or a salt, solvate, stereoisomer, or isotopologue thereof having the structure of Formula (I): 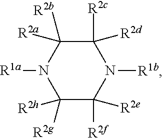
wherein:
R^{1a} and R^{1b} are each independently 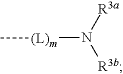
R^{2a}, R^{2b}, R^{2c}, R^{2d}, R^{2e}, R^{2f}, R^{2g}, and R^{2h} are each independently selected from the group consisting of H, optionally substituted C₁-C₁₂ alkyl, optionally substituted C₂-C₁₂ heteroalkyl, optionally substituted C₃-C₈ cycloalkyl, optionally substituted C₂-C₈ heterocycloalkyl, optionally substituted C₂-C₁₂ alkenyl, optionally substituted C₂-C₁₂ alkynyl, optionally substituted C₆-C₁₀ aryl, and optionally substituted C₂-C₁₀ heteroaryl;
each occurrence of R^{3a}, R^{3b}, and R^{3c} is independently selected from the group consisting of H, 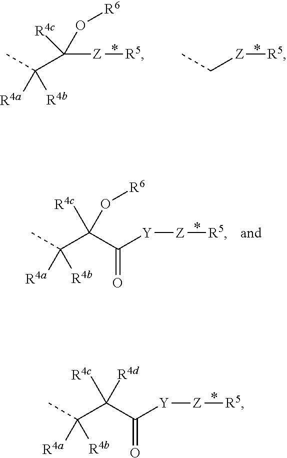
wherein no more than one of R^{3a}, R^{3b}, and R^{3c} is H;
each occurrence of R^{4a}, R^{4b}, R^{4c}, and R^{4d}, if present, is independently selected from the group consisting of optionally substituted C₁-C₁₂ alkyl, halogen, CN, and NO₂;
each occurrence of R⁵ is independently selected from the group consisting of optionally substituted C₁-C₃ alkyl, optionally substituted C₃-C₁₂ cycloalkyl, optionally substituted C₆-C₁₀ aryl, and optionally substituted C₂-C₁₀ heteroaryl;
each occurrence of R⁶ is independently selected from the group consisting of H, optionally substituted C₁-C₆ alkyl, optionally substituted C₃-C₁₂ cycloalkyl, C₂-C₁₂ heterocycloalkyl, optionally substituted C₆-C₁₂ aralkyl, optionally substituted C₆-C₁₂ aryl, optionally substituted C₂-C₁₂ heteroaryl, C(═O)R^{a}, C(═O)OR^{a}, and C(═O)N(R^{a})(R^{b});
each occurrence of L is independently selected from the group consisting of a bond, -(optionally substituted C₁-C₁₂ alkylenyl)-X—, -(optionally substituted C₂-C₁₂ alkenylenyl)-X—, -(optionally substituted C₁-C₁₂ alkynylenyl)-X—, -(optionally substituted C₁-C₁₂ heteroalkylenyl)-X—, optionally substituted C₃-C₈ cycloalkylenyl, and optionally substituted C₂-C₈ heterocyloalkylenyl;
each occurrence of X, if present, is independently selected from the group consisting of a bond, —N(R^{3c})—, and —O—;
each occurrence of Y, if present, is independently selected from the group consisting of a bond, —N(R^{a})—, and —O—;
each occurrence of Z is C₁-C₂₄ alkylenyl,
wherein the C₁-C₂₄ alkylenyl in each occurrence of Z is independently substituted with at least one substituent selected from the group consisting of C₁-C₁₂ alkyl and C₁-C₁₂ haloalkyl, and
wherein the C₁-C₂₄ alkylenyl in each occurrence of Z is independently optionally further substituted;
each occurrence of R^{a} and R^{b} is independently selected from the group consisting of H, optionally substituted C₁-C₆ alkyl, optionally substituted C₃-C₈ cycloalkyl, optionally substituted C₁-C₆ haloalkyl, optionally substituted C₆-C₁₂ aralkyl, optionally substituted phenyl, optionally substituted C₂-C₈ heteroaryl, C(═O)R^{c}, C(═O)OR^{c}, and C(═O)N(R^{c})(R^{d});
each occurrence of R^{c} and R^{d} is independently selected from the group consisting of H, optionally substituted C₁-C₆ alkyl, optionally substituted C₃-C₈ cycloalkyl, optionally substituted C₁-C₆ haloalkyl, optionally substituted C₆-C₁₂ aralkyl, optionally substituted phenyl, and optionally substituted C₂-C₈ heteroaryl; and
each occurrence of m is independently 1, 2, 3, or and 4;
(b) at least one neutral lipid;
(c) cholesterol; and
(d) at least one conjugated lipid.

16. The LNP of claim 15, wherein the LNP further comprises:
(e) at least one nucleic acid and/or therapeutic agent cargo, wherein the cargo is at least partially encapsulated therein.

17-28. (canceled)

29. The LNP of claim 15, which is selected from the group consisting of:

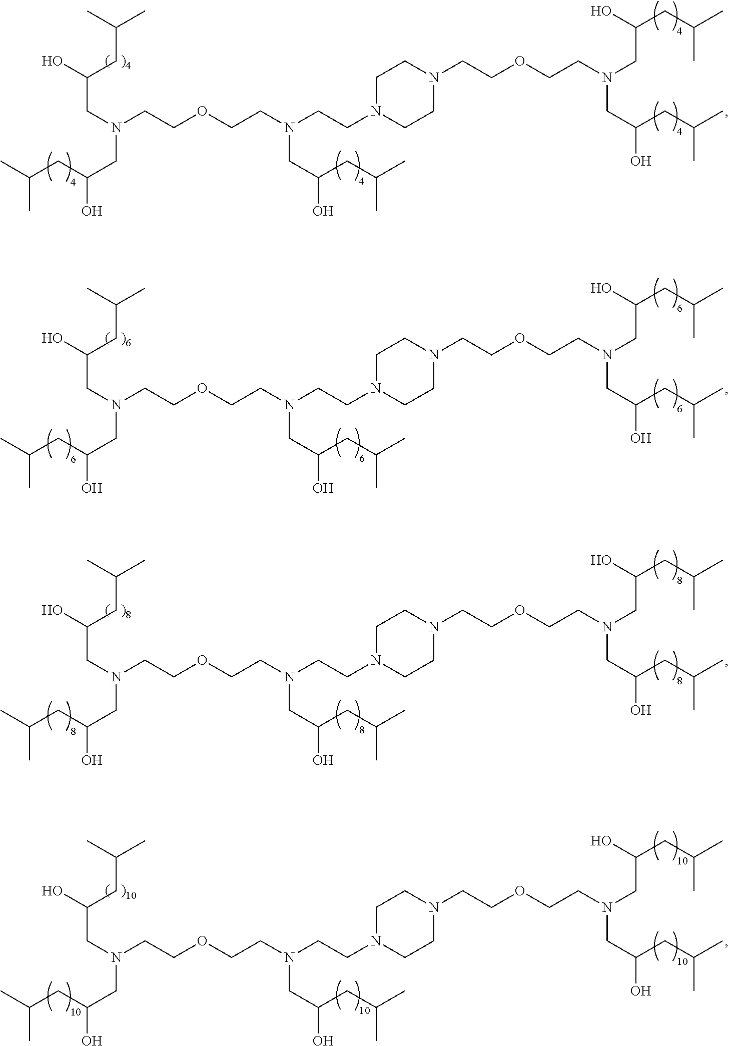

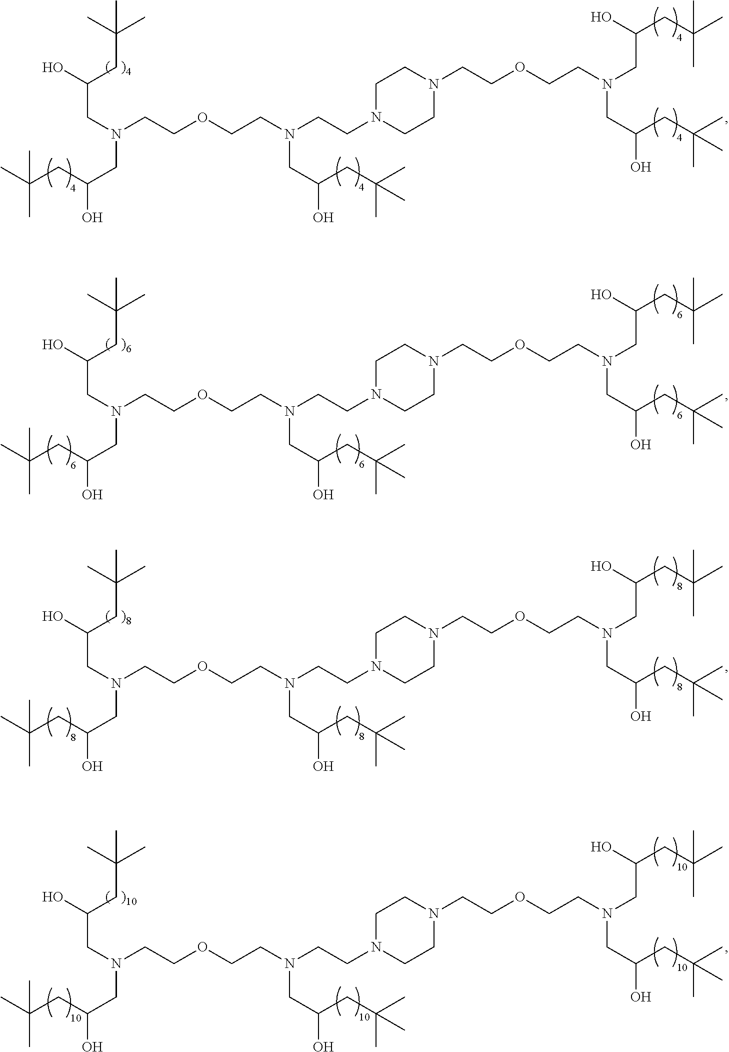

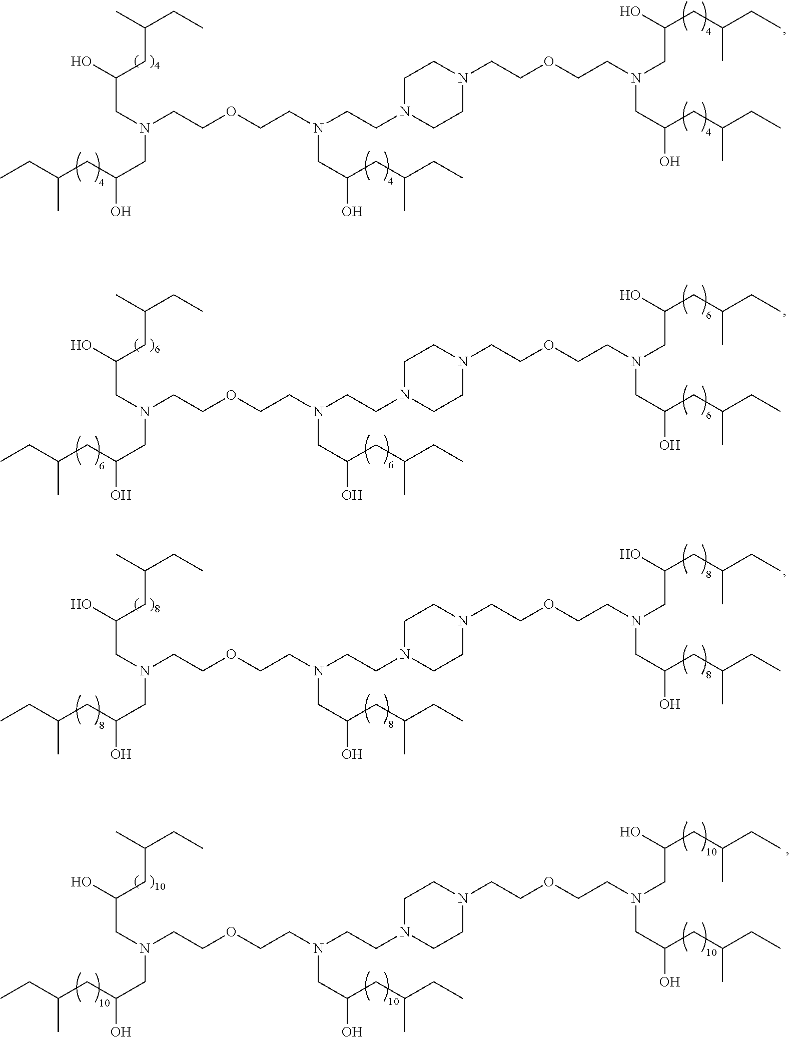

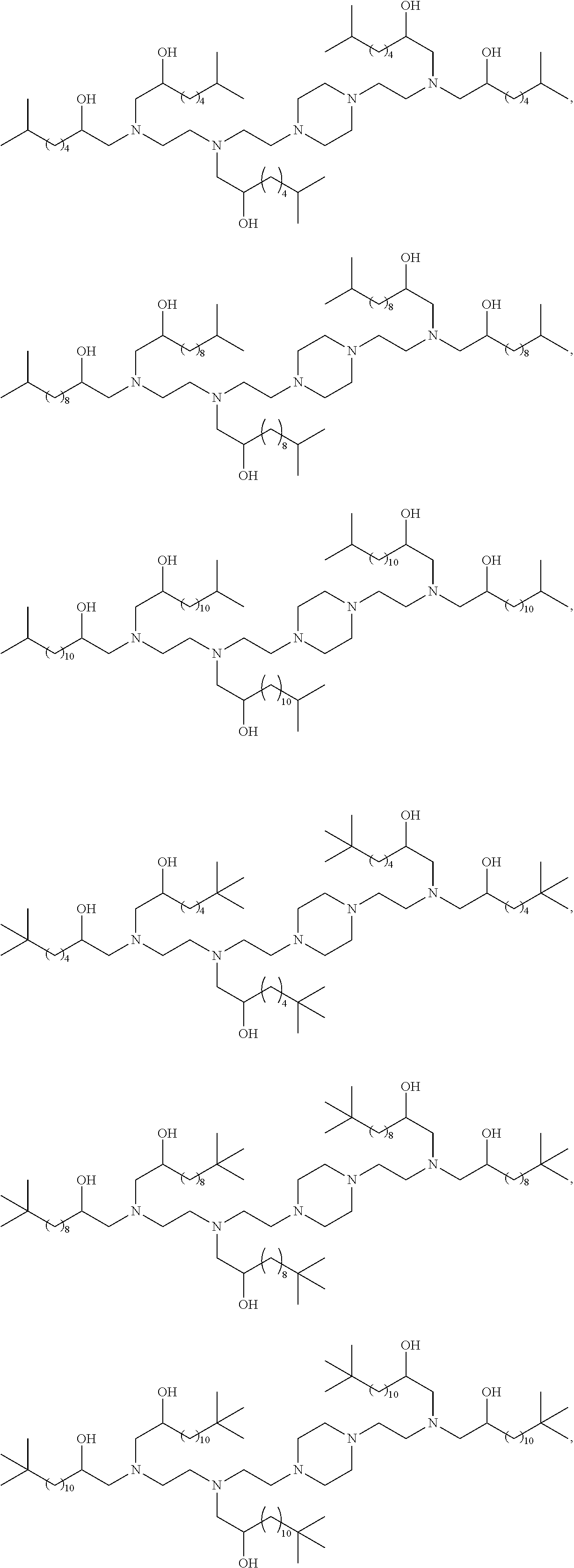

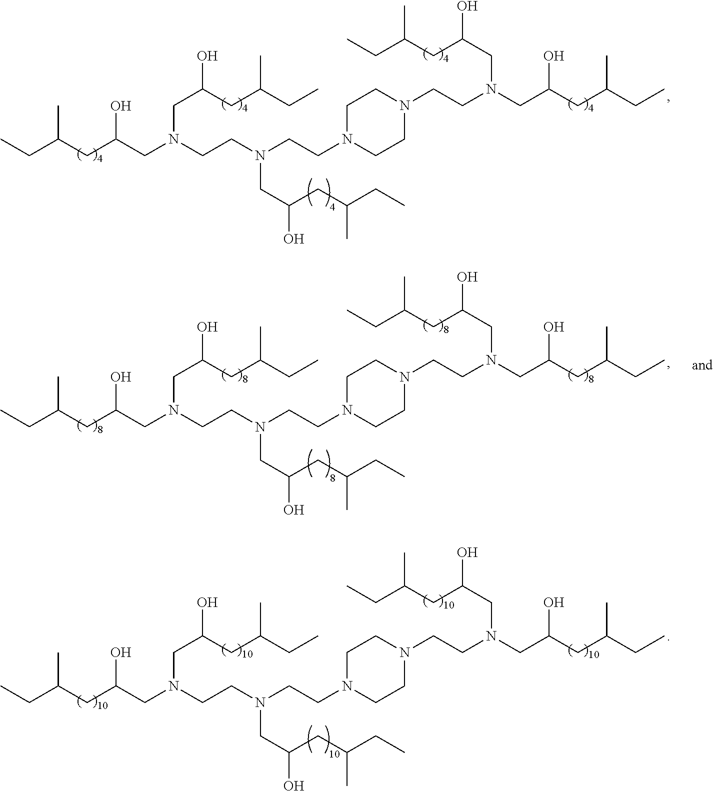

30. The LNP of claim 15, wherein at least one of the following applies:
(a) the at least one ionizable lipid of Formula (I) comprises about 10 mol % to about 90 mol % of the LNP, optionally wherein the at least one ionizable lipid of Formula (I) comprises about 35 mol % or about 40 mol % of the LNP,
(b) the at least one neutral lipid comprises about 1 mol % to about 40 mol % of the LNP, optionally wherein the at least one neutral lipid comprises about 16 mol % or about 30 mol % of the LNP,
(c) the at least one neutral lipid comprises at least one selected from the group consisting of dioleoylphosphatidylethanolamine (DOPE), distearoylphosphatidylcholine (DSPC), and dioleoylphosphatidylcholine (DOPC);
(d) the cholesterol comprises about 20 mol % to about 75 mol % of the LNP, optionally wherein the cholesterol comprises about 25 mol % or about 46.5 mol % of the LNP,
(e) the at least one conjugated lipid comprises about 0.1 mol % to about 15 mol % of the LNP, optionally wherein the at least one conjugated lipid comprises about 2.5 mol % of the LNP;
(f) the at least one conjugated lipid comprises 1,2-dimyristoyl-rac-glycero-3-methoxypolyethylene glycol-2000 (DMG-PEG₂₀₀₀); and
(g) the LNP has a molar ratio of (a):(b):(c):(d) of about 35:16:46.5:2.5 or about 40:30:25:2.5.

31-41. (canceled)

42. The LNP of claim 16, wherein at least one of the following applies:
(a) the nucleic acid molecule is a therapeutic agent; and
(b) the nucleic acid molecule is at least one selected from the group consisting of RNA and DNA, optionally wherein the nucleic acid molecule is at least one selected from the group consisting of mRNA, cDNA, miRNA, siRNA, and modified RNA.

43-45. (canceled)

46. The LNP of claim 42, wherein the LNP has a mass ratio of (a):mRNA of about 20:1 to about 5:1 (w/w), optionally wherein the LNP has a mass ratio of (a):mRNA of about 10:1.

47. The LNP of claim 42, wherein at least one of the following applies:
(a) the mRNA encodes a chimeric antigen receptor (CAR), optionally wherein the CAR is specific for binding to a surface antigen of a pathogenic cell or a tumor cell, and optionally wherein the surface antigen is selected from the group consisting of CD1, CD2, CD3, CD5, CD7, CD8, CD16, CD19, CD20, CD22, CD25, CD26, CD27, CD28, CD30, CD33, CD38, CD39, CD40L, CD44, CD45, CD62L, CD69, CD73, CD80, CD83, CD86, CD95, CD103, CD119, CD123, CD126, CD150, CD153, CD154, CD161, CD183, CD223, CD254, CD275, CD45RA, CXCR3, CXCR5, FasL, IL18R1, CTLA-4, OX40, GITR, LAG3, ICOS, PD-1, leu-12, TCR, TLR1, TLR2, TLR3, TLR4, TLR6, NKG2D, CCR, CCR1, CCR2, CCR4, CCR6, CCR7, k light chain, ROR1, ErbB2, ErbB3, ErbB4, EGFR vIII, carcinoembryonic antigen, EGP2, EGP40, mesothelin, TAG72, PSMA, NKG2D ligands, B7-H6, IL13R-α2, MUC1, VEGF-A, Tem8, FAP, EphA2, HER2, MUC16, CA9, GD2, GD3, HMW-MAA, CD171, Lewis Y, G250/CALX, HLA-AI MAGE A1, HAL-A2 NY-ESO-1, PSC1, folate receptor-α, 8H9, NCAM, VEGF, 5T4, Fetal AchR, NKG2D ligands, TEM1, and TEM8;
(b) the mRNA encodes an enzyme;
(c) the mRNA encodes a clustered regularly interspaced short palindrome repeats (CRISPR) associated protein, optionally wherein the CRISPR associated protein is Cas9.

48-51. (canceled)

52. A pharmaceutical composition comprising the lipid nanoparticle (LNP) of claim 15 and at least one pharmaceutically acceptable carrier, optionally wherein the composition further comprises at least one adjuvant.

53. (canceled)

54. A method of treating, preventing, and/or ameliorating a disease in a subject, the method comprising administering to the subject at least one lipid nanoparticle (LNP) of claim 16 or a pharmaceutical composition thereof.

55. The method of claim 54, wherein at least one of the following applies:
(a) the disease is selected from the group consisting of cancer, an autoimmune disorder, cardiovascular disease, and neurological disease, optionally wherein the cancer is at least one selected from the group consisting of oral cancer, pancreatic cancer, colorectal cancer, bladder cancer, breast cancer, prostate cancer, renal cancer, hepatocellular cancer, lung cancer, ovarian cancer, cervical cancer, gastric cancer, esophageal cancer, head and neck cancer, melanoma, neuroendocrine cancer, CNS cancer, brain cancer, bone cancer, soft tissue sarcoma, non-small cell lung cancer, small-cell lung cancer, or colon cancer;
(b) the subject is further administered at least one additional agent or therapy useful for treating, preventing, and/or ameliorating cancer in the subject; and
(c) the subject is a mammal, optionally wherein the mammal is a human.

56-59. (canceled)

60. A method of delivering a nucleic acid or therapeutic agent to the liver of a subject, the method comprising administering to the subject at least one lipid nanoparticle (LNP) of claim 16 or a pharmaceutical composition thereof.

61. A method of preparing a modified immune cell or precursor thereof, the method comprising contacting an immune cell or precursor thereof with the lipid nanoparticle (LNP) of claim 16 or a pharmaceutical composition thereof.

62. The method of claim 61, wherein the modified immune cell or precursor thereof is αβ T cell, a γδ T cell, a CD8+ T cell, a CD4+ helper T cell, a CD4+ regulatory T cell, an NK T cell, an NK cell, and any combination thereof, optionally wherein one of the following applies:
(a) the modified immune cell or precursor thereof is a T cell, optionally wherein the T cell is a CD4+ T cell; and
(b) the modified immune cell or precursor thereof is a NK cell.

63-64. (canceled)


SEQUENCE LISTING (3 sequences)

SEQ ID NO: 1 - RNA, 99 nt, synthetic construct
  source 1..99: mol_type = other RNA
ttacagccacgtctacagcagttttagagctagaaatagcaagtaaaataaggctagtccgttatcaacttgaaaaagtggcaccgagtcggtgctttt

SEQ ID NO: 2 - DNA, 21 bp, synthetic construct
  source 1..21: mol_type = other DNA
cggtttactctgacccatttc

SEQ ID NO: 3 - DNA, 20 bp, synthetic construct
  source 1..20: mol_type = other DNA
gggctttctacaagcttacc